(19) United States
(12) Patent Application Publication
(10) Pub. No.: US 2025/0092351 A1
(43) Pub. Date: Mar. 20, 2025

(54) SYSTEMS AND METHODS FOR MANUFACTURING OF THERAPEUTIC CELLS

(71) Applicant: CYTONUS THERAPEUTICS, INC., Carlsbad, CA (US)

(72) Inventors: Remo MOOMIAIE, San Diego, CA (US); Richard KLEMKE, San Diego, CA (US); Yuval ZUR, San Diego, CA (US)

(21) Appl. No.: 18/632,925

(22) Filed: Apr. 11, 2024

Related U.S. Application Data
(63) Continuation of application No. PCT/US2022/046436, filed on Oct. 12, 2022.
(60) Provisional application No. 63/254,920, filed on Oct. 12, 2021.
(60) Provisional application No. 63/325,070, filed on Mar. 29, 2022.

(51) Int. Cl. C12M 1/00; C12N 5/00; C12N 5/077; C12N 5/0775; C12N 5/0783; C12N 5/0786

(52) U.S. Cl. CPC C12M 45/05; C12N 5/0081; C12N 5/0645; C12N 5/0646; C12N 5/0656; C12N 5/0668

(57) ABSTRACT

Described herein are methods for obtaining enucleated cells from nucleated cells. Also described herein are methods for cell processing, including providing a composition containing nucleated cells and enucleating at least a portion of the nucleated cells to produce an enucleated cell fraction. Also described herein are methods for cell processing, including expressing the heterologous gene product. Also provided are pharmaceutical compositions comprising an enucleated cell.

### CROSS-REFERENCE

This application is a continuation application of International Application No. PCT/US2022/046436, filed Oct. 12, 2022, which claims the benefit of U.S. Provisional Application Ser. No. 63/254,920, filed on Oct. 12, 2021, and U.S. Provisional Application Ser. No. 63/325,070, filed on Mar. 29, 2022, each of which is hereby incorporated by reference in its entirety.

### SUMMARY

Described herein, in some aspects, is a method for cell processing, the method comprising: a) providing a composition comprising nucleated cells; and b) enucleating a portion of the nucleated cells to produce an enucleated cell fraction using continuous flow centrifugation.

In some embodiments, the portion of the nucleated cells comprises greater than or equal to about 95% of the nucleated cells. In some embodiments, the composition provided in a) has a volume comprising between more than or equal to about 500 mL to about 10000 mL. In some embodiments, the continuous flow centrifugation generates a density gradient that separates the enucleated cell fraction from the nucleated cells in the composition. In some embodiments, the density gradient comprises a polysaccharide density gradient. In some embodiments, the density gradient comprises at least two, at least three, at least four, at least five, at least six, or at least seven ranges of the density gradient. In some embodiments, the polysaccharide density gradient comprises about 25% polysaccharide, about 17% polysaccharide, about 16% polysaccharide, about 15% polysaccharide, or about 12.5% polysaccharide. In some embodiments, the enucleated cell fraction produced by performing the continuous flow centrifugation once comprises more than or equal to about: 6×10⁷ of enucleated cells to 250×10⁷ of enucleated cells. In some embodiments, the enucleating in b) further comprises generating the density gradient comprising centrifuging a polysaccharide at a maximum centrifugal force of between about 30000 RCF to about 200000 RCF. In some embodiments, the continuous flow centrifugation generates zonal centrifugation for separating at least one enucleated cell from the nucleated cells. In some embodiments, the zonal centrifugation separates the at least one enucleated cell from the nucleated cells based on size of the at least one enucleated cell. In some embodiments, the zonal centrifugation separates the at least one enucleated cell from the nucleated cells based on mass of the at least one enucleated cell. In some embodiments, at least one density fraction is obtained from the density gradient, wherein the at least one density fraction comprises a mixed population of a subset of the nucleated cells and enucleated cells of the enucleated cell fraction. In some embodiments, the mixed population comprises at least 70% of the enucleated cells. In some embodiments, using the continuous flow centrifugation increases a yield of obtaining enucleated cells from nucleated cells by at least 0.1 fold, 0.2 fold, 0.5 fold, 1.0 fold, 2.0 fold, 5.0 fold, 10.0 fold, or more fold compared to a method of obtaining the enucleated cells from the nucleated cells by a method without using the continuous flow centrifugation. In some embodiments, the nucleated cells comprise a heterologous polynucleotide. In some embodiments, the method comprises inducing cell death of the nucleated cells that are not enucleated after b), wherein the cell death is induced by expressing a heterologous gene product encoded by the heterologous polynucleotide in the nucleated cells.

Described herein, in some aspects, is a method for cell processing, the method comprising: a) providing a composition comprising (i) a first subset of nucleated cells, and (ii) enucleated cells derived from a second subset of the nucleated cells, wherein the first subset of the nucleated cells comprises a heterologous polynucleotide encoding the heterologous gene product; and b) expressing the heterologous gene product thereby inducing cell death of at least one nucleated cell of the first subset of the nucleated cells. In some embodiments, the heterologous polynucleotide comprises a promoter. In some embodiments, the promoter comprises an inducible promoter. In some embodiments, the promoter comprises a constitutively active promoter. In some embodiments, the heterologous gene product comprises herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase. In some embodiments, the heterologous gene product comprises FKBP or a caspase. In some embodiments, the heterologous gene product comprises an antigen, wherein the antigen induces cell death of the at least one nucleated cell of the first subset of the nucleated cells by triggering an immune response. In some embodiments, the immune response is an in vivo immune response. In some embodiments, the immune response is an in vitro immune response. In some embodiments, the heterologous polynucleotide is integrated into chromosome of the nucleated cells. In some embodiments, the heterologous polynucleotide comprises a vector. In some embodiments, the expressing of the heterologous gene product increases a yield of obtaining enucleated cells from nucleated cells by at least 0.1 fold, 0.2 fold, 0.5 fold, 1.0 fold, 2.0 fold, 5.0 fold, 10.0 fold, or more fold compared to a method of obtaining the enucleated cells from the nucleated cells by a method without expressing of the heterologous gene product. In some embodiments, the method further comprises cryopreserving the enucleated cell fraction to produce a cryopreserved enucleated cell fraction. In some embodiments, the method further comprises thawing the cryopreserved enucleated cell fraction, wherein, following the thawing, an enucleated cell of the cryopreserved enucleated cell fraction is as viable as an otherwise comparable enucleated cell that is not cryopreserved. In some embodiments, the nucleated cells comprise stem cells. In some embodiments, the stem cells comprise induced pluripotent stem cells (iPSCs), adult stem cells, mesenchymal stromal cells, embryonic stem cells, fibroblasts, or immortalized cells from a cell line, or a combination thereof. In some embodiments, the nucleated cells comprise the mesenchymal stromal cells. In some embodiments, the nucleated cells comprise immune cells. In some embodiments, the immune cells comprise lymphocytes or natural killer cells. In some embodiments, the enucleated cells lack a nucleus and comprise one or more intracellular organelles for synthesis or secretion of an exogenous polypeptide in absence of the nucleus. In some embodiments, the exogenous polypeptide is encoded by the heterologous polynucleotide. In some embodiments, the exogenous polypeptide comprises a therapeutic agent. In some embodiments, the enucleated cells comprise at least one targeting moiety. In some embodiments, the enucleated cells comprise at least one fusogenic moiety. In some embodiments, the enucleated cells comprise at least one immune evasion moiety. In some embodiments, an enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, the enucleated cell of the enucleated cell fraction has a diameter comprising more than or equal to about 5 μm, about 10 μm, about 20 μm, about 30 μm, about 40 μm, about 50 μm, about 60 μm, about 70 μm, about 80 μm, or about 90 μm. In some embodiments, the diameter comprises about 8 pm.

Described herein, in some aspects, is a composition comprising: a) enucleated cells obtained from a first subset of a plurality of nucleated cells; and b) a second subset of the plurality of nucleated cells, wherein a nucleated cell of the second subset of the plurality of the nucleated cells comprises a heterologous polynucleotide encoding a heterologous gene product configured to induce cell death of the nucleated cell. In some embodiments, the heterologous polynucleotide comprises a promoter configured to activate transcription of the heterologous polynucleotide under conditions sufficient to express the heterologous gene product. In some embodiments, the promoter comprises an inducible promoter configured to activate transcription of the heterologous polynucleotide under conditions sufficient to express the heterologous gene product when induced. In some embodiments, the promoter comprises a constitutively active promoter. In some embodiments, the constitutively active promoter is configured to activate transcription of the heterologous polynucleotide under conditions sufficient to express the heterologous gene product. In some embodiments, the heterologous gene product comprises herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase. In some embodiments, the heterologous gene product comprises FKBP or a caspase. In some embodiments, the heterologous gene product comprises an antigen, wherein the antigen induces cell death of the at least one nucleated cell of the first subset of the nucleated cells by triggering an immune response. In some embodiments, the immune response is an in vivo immune response. In some embodiments, the immune response is an in vitro immune response. In some embodiments, the heterologous polynucleotide is integrated into chromosome of the nucleated cell. In some embodiments, the heterologous polynucleotide comprises a vector. In some embodiments, the plurality of nucleated cells comprises stem cells. In some embodiments, the stem cells comprise induced pluripotent stem cells (iPSCs), adult stem cells, mesenchymal stromal cells, embryonic stem cells, fibroblasts, or immortalized cells from a cell line, or a combination thereof. In some embodiments, the stem cells comprise the mesenchymal stromal cells. In some embodiments, the plurality of nucleated cells comprises immune cells. In some embodiments, the immune cells comprise lymphocytes or natural killer cells. In some embodiments, the enucleated cells lack a nucleus and comprise one or more structural features of the plurality of nucleated cells. In some embodiments, the one or more structural features comprise one or more intracellular organelles, one or more tunneling nanotubes, or a combination thereof. In some embodiments, the enucleated cells lack a nucleus and comprise one or more intracellular organelles for synthesis or secretion of an exogenous polypeptide in absence of the nucleus. In some embodiments, the one or more intracellular organelles comprise a Golgi apparatus, an endoplasmic reticulum, or a combination thereof. In some embodiments, the exogenous polypeptide comprises a therapeutic agent. In some embodiments, the enucleated cells comprise at least one targeting moiety. In some embodiments, the enucleated cells comprise at least one fusogenic moiety. In some embodiments, the enucleated cells comprise at least one immune evasion moiety. In some embodiments, the enucleated cells comprise at least one therapeutic moiety. In some embodiments, the enucleated cell of an enucleated cell fraction has a diameter comprising less than or equal to at least about 10%, about 20%, about 30%, about 40%, about 50%, about 60%, about 70%, about 80%, about 90%, about 95%, or about 99% of an average diameter of the nucleated cells. In some embodiments, the enucleated cell of the enucleated cell fraction has a diameter comprising between about 10 μm to about 100 μm. In some embodiments, the diameter comprises about 8 μm. In some embodiments, the composition is in a dosage form suitable for intravenous administration. In some embodiments, the dosage form comprises a solid dosage form. In some embodiments, the composition comprises a tablet, a pill, a powder, a capsule, solid dispersion, solid solution, bioerodible dosage form, a controlled release formulation, a pulsatile release dosage form, a multiparticulate dosage form, a bead, a pellet, or a granule. In some embodiments, the enucleated cell is further cryopreserved to produce a cryopreserved enucleated cell. In some embodiments, the cryopreserved enucleated cell fraction is thawed, wherein, following the thawing, the enucleated cell of the cryopreserved enucleated cell fraction is as viable as an otherwise comparable enucleated cell that is not cryopreserved. In some embodiments, the enucleated cell exhibits viability after cryohibemation. In some embodiments, the enucleated cell exhibits the viability following the cryohibernation as measured at 24 hours following the cryohibernation that is equal to or greater than the viability of a comparable enucleated cell that is not cryohibernated. In some embodiments, the enucleated cell exhibits viability after cryopreservation. In some embodiments, the enucleated cell exhibits the viability following the cryopreservation as measured at 24 hours following the cryopreservation that is equal to or greater than the viability of a comparable enucleated cell that is not cryopreserved. In some embodiments, the composition is purified. In some embodiments, the composition is lyophilized. In some embodiments, the enucleated cells and the plurality of the nucleated cells are at the same stage of cell differentiation. In some embodiments, the enucleated cells are not obtained from the plurality of the nucleated cells by cell differentiation. In some embodiments, the enucleated cells are not terminally differentiated cells. In some embodiments, the enucleated cells are not platelets. In some embodiments, the enucleated cells are not obtained from platelet lineage cells. In some embodiments, the enucleated cells are not red blood cells. In some embodiments, the enucleated cells are not obtained from red blood cell lineage cells.

Described herein, in some aspects, is a plurality of enucleated cells comprising a plurality of the enucleated cells disclosed herein.

Described herein, in some aspects, is a pharmaceutical composition comprising: a) the enucleated cells disclosed herein; and b) a pharmaceutically acceptable: excipient, carrier, or diluent. In some embodiments, the pharmaceutical composition is in a unit dose form. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof, to a subject. In some embodiments, the pharmaceutical composition is formulated for administering intravenously. In some embodiments, the pharmaceutical composition further comprises at least one additional active agent. In some embodiments, the at least one additional active agent comprises a cytokine, a growth factor, a hormone, an enzyme, a small molecule, a compound, or a combination thereof.

Described herein, in some aspects, is a kit comprising: a) the composition disclosed herein or the pharmaceutical composition disclosed herein; and b) a container.

Described herein, in some aspects, is a method for cell processing, the method comprising: providing a composition comprising nucleated cells; and enucleating a portion of the nucleated cells to produce an enucleated cell fraction using continuous flow centrifugation, wherein the portion of the nucleated cells comprises greater than or equal to about 70% of the nucleated cells. In some embodiments, the composition provided the method has a volume comprising between more than or equal to about 10 milliliters (mL) to about 10000 mL. In some embodiments, the composition has a volume comprising more than or equal to about 10 milliliter (mL), about 20 mL, about 30 mL, about 40 mL, about 50 mL, about 60 mL, about 80 mL, about 100 mL, about 200 mL, about 300 mL, about 500 mL, about 1000 mL, about 2000 mL, about 3000 mL, about 4000 mL, about 5000 mL, about 6000 mL, about 7000 mL, about 8000 mL, about 9000 mL, or about 10000 mL. In some embodiments, the continuous flow centrifugation generates a density gradient that separates the enucleated cell fraction from the nucleated cells in the composition. In some embodiments, the density gradient comprises a polysaccharide density gradient. In some embodiments, the polysaccharide density gradient comprises a Ficoll density gradient. In some embodiments, the Ficoll density gradient comprises at least two, at least three, at least four, at least five, at least six, or at least seven ranges of the density gradient. In some embodiments, the Ficoll density gradient comprises about 25% Ficoll, about 17% Ficoll, about 16% Ficoll, about 15% Ficoll, or about 12.5% Ficoll. In some embodiments, the portion of the nucleated cells comprises greater than or equal to about 75% of the nucleated cells. In some embodiments, the portion of the nucleated cells comprises greater than or equal to about 80% of enucleated cells. In some embodiments, the portion of the nucleated cells comprises greater than or equal to about 90% of enucleated cells. In some embodiments, the enucleated cell fraction produced by performing the continuous flow centrifugation once comprises more than or equal to about: (i) 6×10⁷ of enucleated cells, (ii) 7×10⁷ of enucleated cells, (iii) 8×10⁷ of enucleated cells, (iv) 9×10⁷ of enucleated cells, (v) 10×10⁷ of enucleated cells, (vi) 15×10⁷ of enucleated cells, (vii) 20×10⁷ of enucleated cells, (viii) 50×10⁷ of enucleated cells, (ix) 100×10⁷ of enucleated cells, (x) 150×10⁷ of enucleated cells, (xi) 200×10⁷ of enucleated cells, or (xii) 250×10⁷ of enucleated cells. In some embodiments, the method comprises generating the density gradient comprising centrifuging a polysaccharide with acceleration spanning over at least about 10 minutes, at least about 20 minutes, at least about 30 minutes, at least about 40 minutes, or at least about 50 minutes. In some embodiments, the method comprises generating the density gradient comprising centrifuging a polysaccharide with acceleration spanning over about 10 minutes, about 20 minutes, about 30 minutes, about 40 minutes, or about 50 minutes. In some embodiments, the method comprises, generating the density gradient comprising centrifuging a polysaccharide with acceleration spanning about 30 minutes. In some embodiments, the enucleating of the method further comprises generating the density gradient comprising centrifuging a polysaccharide with minimal deceleration. In some embodiments, the enucleating of the method further comprises generating the density gradient comprising centrifuging a polysaccharide at a maximum centrifugal force of between about 30000 relative centrifugal force (RCF) to about 200000 RCF. In some embodiments, the enucleating of the method further comprises generating the density gradient comprising centrifuging a polysaccharide at a maximum centrifugal force of between about 50000 RCF to about 120000 RCF. In some embodiments, enucleating the portion of the nucleated cells to produce the enucleated cell fraction using the continuous flow centrifugation in the method is performed using an ultracentrifuge. In some embodiments, enucleating the portion of the nucleated cells to produce the enucleated cell fraction using the continuous flow centrifugation is performed using fixed angle centrifugation or swinging bucket centrifugation. In some embodiments, the nucleated cells comprise a heterologous polynucleotide. In some embodiments, the method further comprises inducing cell death of the nucleated cells that are not enucleated, where the cell death is induced by expressing at least one heterologous gene encoded by the heterologous polynucleotide. In some embodiments, the continuous flow centrifugation generates zonal centrifugation for separating at least one enucleated cell from the nucleated cells. In some embodiments, the zonal centrifugation separates the at least one enucleated cell from the nucleated cells based on size of the at least one enucleated cell. In some embodiments, the zonal centrifugation separates the at least one enucleated cell from the nucleated cells based on mass of the at least one enucleated cell. In some embodiments, the zonal centrifugation separates the at least one enucleated cell from the nucleated cells based on size and mass of the at least one enucleated cell. In some embodiments, at least one density fraction is obtained from the density gradient, wherein the at least one density fraction comprises a mixed population of a subset of the nucleated cells and enucleated cells of the enucleated cell fraction. In some embodiments, the mixed population comprises at least 70% of the enucleated cells. In some embodiments, the mixed population comprises at least 99% of the enucleated cells.

Described herein, in some embodiments, is a method for cell processing, the method comprising: providing a composition comprising: a first subset of nucleated cells; and enucleated cells derived from a second subset of said nucleated cells, where said first subset of said nucleated cells comprises a heterologous polynucleotide encoding a heterologous gene product; and expressing said heterologous gene product thereby inducing cell death of at least one nucleated cell of said first subset of said nucleated cells. In some embodiments, the heterologous polynucleotide comprises a promoter. In some embodiments, the promoter comprises an inducible promoter. In some embodiments, the inducible promoter is induced by contacting the nucleated cells with a temperature that is below 37° C. In some embodiments, the inducible promoter comprises dsrA or CIRP. In some embodiments, the inducible promoter is induced by contacting the nucleated cells with a temperature that is above 37° C. In some embodiments, the inducible promoter comprises heat shock protein 70 (HSP70, e.g., NCBI Gene ID 3308), heat shock protein 90 (HSP90, e.g., NCBI Gene ID 3320), growth arrest- and DNA damage-inducible gene 153 (GADD153, e.g., NCBI Gene ID 1649), multidrug resistance mutation 1 (MDR1, e.g., NCBI Gene ID 5243), or cytomegalovirus (HSE-CMV, e.g., NCBI Gene ID 3077513). In some embodiments, the inducible promoter is induced by contacting the nucleated cells with a molecule. In some embodiments, the molecule comprises rtTA, TRE, TetR, Cumate, Rapamycin, Abscisic acid, IPTG, or Methallothionein. In some embodiments, the inducible promoter is induced by contacting the nucleated cells with light. In some embodiments, the inducible promoter comprises CIB1-CRY2 or GAL4-VVD. In some embodiments, the inducible promoter is induced by contacting with the nucleated cells with a hormone. In some embodiments, the inducible promoter comprises Estradiol-Gal4. In some embodiments, the promoter comprises a constitutively active promoter. In some embodiments, the constitutively active promoter is configured to activate transcription of the heterologous polynucleotide under conditions sufficient to express the heterologous gene product. In some embodiments, the heterologous gene product comprises herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase. In some embodiments, the heterologous gene product comprises FKBP or a caspase. In some embodiments, the heterologous gene product comprises an antigen. In some embodiments, the heterologous polynucleotide is integrated into chromosome of the nucleated cells. In some embodiments, the heterologous polynucleotide comprises a vector. In some embodiments, the method further comprises cryopreserving the enucleated cell fraction to produce a cryopreserved enucleated cell fraction. In some embodiments, the method further comprises thawing the cryopreserved enucleated cell fraction, wherein, following the thawing, an enucleated cell of the cryopreserved enucleated cell fraction is as viable as an otherwise comparable enucleated cell that was not cryopreserved. In some embodiments, the nucleated cells comprise stem cells. In some embodiments, the stem cells comprise induced pluripotent stem cells (iPSCs), adult stem cells, mesenchymal stromal cells, embryonic stem cells, fibroblasts, or immortalized cells from a cell line, or a combination thereof. In some embodiments, the nucleated cells comprise the mesenchymal stromal cells. In some embodiments, the enucleated cells lack a nucleus and comprise one or more intracellular organelles for synthesis or secretion of an exogenous polypeptide in absence of the nucleus. In some embodiments, the exogenous polypeptide comprises a therapeutic agent. In some embodiments, the enucleated cells comprise at least one targeting moiety. In some embodiments, the enucleated cells comprise at least one fusogenic moiety. In some embodiments, the enucleated cells comprise at least one immune evasion moiety. In some embodiments, the enucleated cells comprise at least one therapeutic moiety. In some embodiments, the enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells provided in the method. In some embodiments, the enucleated cell of the enucleated cell fraction has a diameter comprising less than or equal to about 70% of an average diameter of the nucleated cells provided in the method. In some embodiments, the enucleated cell of the enucleated cell fraction has a diameter comprising more than or equal to about 5 micrometers (pm), about 10 μm, about 20 μm, about 30 μm, about 40 μm, about 50 μm, about 60 μm, about 70 μm, about 80 μm, or about 90 gm. In some embodiments, the enucleated cell of the enucleated cell fraction comprises a diameter comprising between about 10 gm to about 100 gm. In some embodiments, the diameter comprises about 8 μm.

Described herein, in some aspects, is a composition comprising: enucleated cells obtained from a first subset of a plurality of nucleated cells; and a second subset of the plurality of nucleated cells, wherein a nucleated cell of the second subset of the plurality of the nucleated cells comprises a heterologous polynucleotide encoding a heterologous gene product configured to induce cell death of the nucleated cell. In some embodiments, the heterologous polynucleotide comprises a promoter configured to activate transcription of the heterologous polynucleotide under conditions sufficient to express the heterologous gene product. In some embodiments, the promoter comprises an inducible promoter configured to activate transcription of the heterologous polynucleotide under conditions sufficient to express the heterologous gene product when induced. In some embodiments, the inducible promoter is induced by contacting the nucleated cells with a temperature that is below 37° C. In some embodiments, the inducible promoter comprises dsrA or CIRP. In some embodiments, the inducible promoter is induced by contacting the nucleated cell with a temperature that is above 37° C. In some embodiments, the inducible promoter comprises heat shock protein 70 (HSP70, e.g., NCBI Gene ID 3308), heat shock protein 90 (HSP90, e.g., NCBI Gene ID 3320), growth arrest- and DNA damage-inducible gene 153 (GADD153, e.g., NCBI Gene ID 1649), multidrug resistance mutation 1 (MDR1, e.g., NCBI Gene ID 5243), or cytomegalovirus (HSE-CMV, e.g., NCBI Gene ID 3077513). In some embodiments, the inducible promoter is induced by contacting the nucleated cell with a molecule. In some embodiments, the molecule comprises rtTA, TRE, TetR, Cumate, Rapamycin, Abscisic acid, IPTG, or Methallothionein. In some embodiments, the inducible promoter is induced by contacting the nucleated cell with light. In some embodiments, the inducible promoter comprises CIB1-CRY2 or GAL4-VVD. In some embodiments, the inducible promoter is induced by contacting the nucleated cell with a hormone. In some embodiments, the inducible promoter comprises Estradiol-Gal4. In some embodiments, the promoter comprises a constitutively active promoter. In some embodiments, the constitutively active promoter is configured to activate transcription of the heterologous polynucleotide under conditions sufficient to express the heterologous gene product. In some embodiments, the heterologous gene product comprises herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase. In some embodiments, the heterologous gene product comprises FKBP or a caspase. In some embodiments, the heterologous gene product comprises an antigen. In some embodiments, the heterologous polynucleotide is integrated into chromosome of the nucleated cell. In some embodiments, the heterologous polynucleotide comprises a vector.

Described herein, in some aspects, is a composition comprising: enucleated cells obtained from a first subset of a plurality of nucleated cells, wherein less than or equal to about 0.1% by volume of the composition further comprises a second subset of the plurality of the nucleated cells. In some embodiments, the plurality of nucleated cells comprises stem cells. In some embodiments, the stem cells comprise induced pluripotent stem cells (iPSCs), adult stem cells, mesenchymal stromal cells, embryonic stem cells, fibroblasts, or immortalized cells from a cell line, or a combination thereof. In some embodiments, the nucleated cells comprise the mesenchymal stromal cells. In some embodiments, the enucleated cells lack a nucleus and comprise one or more structural features of the plurality of nucleated cells. In some embodiments, the one or more structural features comprise one or more intracellular organelles, one or more tunneling nanotubes, or a combination thereof. In some embodiments, the enucleated cells lack a nucleus and comprise one or more intracellular organelles for synthesis or secretion of an exogenous polypeptide in absence of the nucleus. In some embodiments, the one or more intracellular organelles comprise a Golgi apparatus, an endoplasmic reticulum, or a combination thereof. In some embodiments, the exogenous polypeptide comprises a therapeutic agent. In some embodiments, the enucleated cells comprise at least one targeting moiety. In some embodiments, the enucleated cells comprise at least one fusogenic moiety. In some embodiments, the enucleated cells comprise at least one immune evasion moiety. In some embodiments, the enucleated cells comprise at least one therapeutic moiety. In some embodiments, the enucleated cell of an enucleated cell fraction has a diameter comprising less than or equal to at least about 10%, about 20%, about 30%, about 40%, about 50%, about 60%, about 70%, about 80%, about 90%, about 95%, or about 99% of an average diameter of the nucleated cells provided in the composition. In some embodiments, the enucleated cell of the enucleated cell fraction has a diameter comprising less than or equal to about 70% of an average diameter of the nucleated cells provided in the composition. In some embodiments, the enucleated cell of the enucleated cell fraction has a diameter comprising between about 10 μm to about 100 μm. In some embodiments, the enucleated cell of the enucleated cell fraction has a diameter comprising more than or equal to about 1 μm, about 5 μm, about 8 μm, about 10 μm, about 20 μm, about 30 μm, about 40 μm, about 50 μm, about 60 μm, about 70 μm, about 80 μm, about 90 μm, or about 100 μm. In some embodiments, the diameter comprises about 8 μm. In some embodiments, the composition is in a dosage form suitable for intravenous administration. In some embodiments, the dosage form comprises a solid dosage form. In some embodiments, the composition comprises a tablet, a pill, a powder, a capsule, solid dispersion, solid solution, bioerodible dosage form, a controlled release formulation, a pulsatile release dosage form, a multiparticulate dosage form, a bead, a pellet, or a granule. In some embodiments, a total number of the enucleated cells in the composition comprises more than or equal to about 10 million enucleated cells, about 20 million enucleated cells, about 30 million enucleated cells, about 40 million enucleated cells, about 45 million enucleated cells, about 50 million enucleated cells, about 55 million enucleated cells, about 60 million enucleated cells, about 65 million enucleated cells, about 70 million enucleated cells, about 75 million enucleated cells, about 80 million enucleated cells, about 90 million enucleated cells, or about 100 million enucleated cells. In some embodiments, the enucleated cell is further cryopreserved to produce a cryopreserved enucleated cell. In some embodiments, the cryopreserved enucleated cell fraction is thawed, wherein, following the thawing, the enucleated cell of the cryopreserved enucleated cell fraction is as viable as an otherwise comparable enucleated cell that was not cryopreserved. In some embodiments, the enucleated cell exhibits viability after cryohibernation. In some embodiments, the enucleated cell exhibits the viability following the cryohibemation as measured at 24 hours following the cryohibernation that is equal to or greater than the viability of a comparable enucleated cell that is not cryohibernated. In some embodiments, the enucleated cell exhibits viability after cryopreservation. In some embodiments, the enucleated cell exhibits the viability following the cryopreservation as measured at 24 hours following the cryopreservation that is equal to or greater than the viability of a comparable enucleated cell that is not cryopreserved. In some embodiments, the composition is purified. In some embodiments, the composition is lyophilized. In some embodiments, the enucleated cells and the plurality of the nucleated cells are at the same stage of cell differentiation. In some embodiments, the enucleated cells are not obtained from the plurality of the nucleated cells by cell differentiation. In some embodiments, the enucleated cells are not terminally differentiated cells. In some embodiments, the enucleated cells are not platelets. In some embodiments, the enucleated cells are not obtained from platelet lineage cells. In some embodiments, the enucleated cells are not red blood cells. In some embodiments, the enucleated cells are not obtained from red blood cell lineage cells.

Described herein, in some aspects, is a plurality of enucleated cells comprising a plurality of the enucleated cells described herein.

Described herein, in some aspects, is a pharmaceutical composition comprising: the enucleated cells described herein; and a pharmaceutically acceptable: excipient, carrier, or diluent. In some embodiments, the pharmaceutical composition is in a unit dose form. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof, to a subject. In some embodiments, the pharmaceutical composition is formulated for administering intravenously. In some embodiments, the pharmaceutical composition comprises at least one additional active agent. In some embodiments, the at least one additional active agent comprises a cytokine, a growth factor, a hormone, an enzyme, a small molecule, a compound, or a combination thereof.

Described herein, in some aspects, is a kit comprising: the composition described herein or the pharmaceutical composition described herein; and a container.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Some novel features of the methods and compositions disclosed herein are set forth in the present disclosure. A better understanding of the features and advantages of the methods and compositions disclosed herein will be obtained by reference to the following detailed description that sets forth illustrative embodiments, in which the principles of the disclosed compositions and methods are utilized, and the accompanying drawings of which:

FIG. 1 illustrates a flow chart showing non-limiting steps of a process for composition or pharmaceutical composition of enucleated cells for delivery of therapeutics, according to an embodiment of the present disclosure.

FIG. 2 illustrates a process for generating the enucleated cells for the delivery of the therapeutic agent according to various embodiments described herein.

FIG. 3 illustrates a timeline for production of the enucleated cells for the delivery of the single-domain antibody according to various embodiments, as compared to a typical biological drug development timeline.

FIG. 4A is a representative graph showing the relative fold change in viable cells or enucleated cells (“cytoplasts”) over time.

FIG. 4B is a representative graph showing the viable cells and cytoplasts after recovery from frozen storage (cryopreservation).

FIG. 4C is a representative graph showing the relative viability of cytoplasts 24 hours after enucleation (fresh cytoplasts) or 24 hours after recovery from frozen storage (cryopreserved) following enucleation, where fresh and cryopreserved cytoplasts are compared to the viability of cytoplasts 4 hours after enucleation. Mean±SEM; n=10.

FIG. 5A is a representative line graph showing the viability of MSC and MSC-derived cytoplasts immediately after recovery from cryohibernation at 4 degrees Celsius for the indicated amounts of time. Viability was assessed in an automated cell count (Cell Countess) using Trypan blue dye exclusion and displayed as a ratio to the number of input cells.

FIG. 5B is a representative bar graph comparing the migrated MSC and MSC-derived cytoplasts in a Boyden chamber assay immediately after recovery from cryohibernation at 4 degrees Celsius for the indicated amounts of time. Cells and cytoplasts were allowed to migrate for 3 hours with either no serum (negative control) or 10% premium FBS (P-FBS) as a chemoattractant in the bottom chamber, and counts were normalized to loading controls.

FIG. 6A is a representative flow cytometry graphs showing the number of events counted over the signal strength of the cell surface CXCR4 expression by fluorescent antibody on engineered cytoplasts and engineered parental MSCs as analyzed by FlowJo.

FIG. 6B is a representative bar graph showing the ratio of migrating cells or cytoplasts that migrated to the undersurface of the Boyden chamber membrane compared to the loading control. Mean±SEM; n=10. MSCs and MSC-derived cytoplasts with and without engineered CXCR4 receptors were allowed to migrate towards the indicated concentrations of SDF-1α for 2 hours in a Boyden chamber assay.

FIG. 7A is a representative flow cytometry graph showing the number of events counted over the signal strength of the cell surface PSGL1 expression by fluorescent antibody on engineered cytoplasts and engineered parental MSCs as analyzed by FlowJo.

FIG. 7B is a representative graph showing cell surface binding of P-Selectin with engineered MSCs and MSC-derived cytoplasts as determined by flow cytometry. MSC control=parental MSCs. Engineered MSC=PSGL1/Fut7 engineered MSC. Engineered cytoplast=PSGL1/Fut7 engineered MSC-derived cytoplasts.

FIG. 8A is a representative flow cytometry graph showing the number of events counted over the signal strength of the cell surface of mCD47 expression on engineered cytoplasts and MSCs as analyzed by FlowJo.

FIG. 8B is a representative bar graph showing the number of live cytoplasts (DiD+) that were not phagocytosed by macrophages (F4/80⁻ and CD11b⁻), indicating that cytoplasts escaped macrophage phagocytosis in the lung. Mean±SEM; n=3. DiD dye-labeled Control cytoplasts or engineered cytoplasts (mCD47 Cytoplasts) were retro-orbitally injected into the vasculature of mice. After 24 hours, tissues were harvested and stained with two different pan-macrophage markers (F4/80 and CD11b).

FIG. 8C is a representative bar graph showing live cytoplasts (DiD+) that were not phagocytosed by macrophages (F4/80⁻ and CD11b⁻), indicating that cytoplasts escaped macrophage phagocytosis in the liver. Mean±SEM; n=3. DiD dye-labeled Control cytoplasts or engineered cytoplasts (mCD47 Cytoplasts) were retro-orbitally injected into the vasculature of mice. After 24 hours, tissues were harvested and stained with two different pan-macrophage markers (F4/80 and CD11b).

FIG. 9A is a representative scatter plot showing the number of DiD-labeled MSCs or cytoplasts detected in the lung. MSCs were cultured under standard adherent conditions (2D) or in suspension by the handing drop method (3D) to generate 3D cytoplasts. MSCs and cytoplasts were labeled with Vybrant® DiD dye and retro-orbitally injected into the vasculature of C57BL/6 mice. Tissues were harvested after 24 hours, and cell suspensions analyzed by flow cytometry. Mean±SEM; n=2.

FIG. 9B is a representative scatter plot showing the number of DiD-labeled MSCs or cytoplasts detected in the liver. MSCs were cultured under standard adherent conditions (2D) or in suspension by the handing drop method (3D) to generate 3D cytoplasts. MSCs and cytoplasts were labeled with Vybrant® DiD dye and retro-orbitally injected into the vasculature of C57BL/6 mice. Tissues were harvested after 24 hours, and cell suspensions analyzed by flow cytometry. Mean±SEM; n=2.

FIG. 9C is a representative scatter plot showing the number of Vybrant® DiD-labeled MSCs or cytoplasts detected in the spleen. MSCs were cultured under standard adherent conditions (2D) or in suspension by the handing drop method (3D) to generate 3D cytoplasts. MSCs and cytoplasts were labeled with DiD dye and retro-orbitally injected into the vasculature of C57BL/6 mice. Tissues were harvested after 24 hours, and cell suspensions analyzed by flow cytometry. Mean±SEM; n=2.

FIG. 10 illustrates cell surface staining of fluorescein isothiocyanate (FITC) labeled Annexin V on mesenchymal stromal cells (MSCs) or the cytoplasts analyzed by flow cytometry for cell viability analysis.

FIG. 11A illustrate an exemplary gradient generated by the method described herein.

FIG. 11B illustrates exemplary images of the viable enucleated cells obtained from the nucleated cells. FIG. 11B further illustrates exemplary diameter decrease of the cell due to enucleation. The diameter of the cell is decreased after enucleation (from 18.37 μm to 15.36 μm).

FIG. 11C illustrates that the viability of the nucleated cells was not significantly changed before or after enucleation. FIG. 11C also illustrates enucleation efficiency and viability of the cells after enucleation via the method described herein.

FIG. 11D illustrates fluorescent images of the cells directly after enucleation (top two images) and 24 hours after enucleation (bottom image).

FIG. 12A illustrates density gradient measured after a continuous flow centrifugation (done by ultracentrifugation run (5 Ficoll layers in medium gray; 3 Ficoll layers in dark gray; and continuous flow as indicated by call-out line).

FIG. 12B illustrate representative images of enucleation efficiency test. Each field image was taken using bright field (total cells) and Hoechst channels (total nucleated cells).

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Existing techniques of displacing nuclei in adherent cells, or enucleation, using centrifugal force have drawbacks rending them inoperable for large scale of an enucleated cell platform for biomedical applications. Such drawbacks include, without limitation, small capacities of swinging buckets, ranging from a few milliliters to a few hundred milliliters, and long pathlengths for overall pelleting time, resulting in small-scale production with low efficiency. These drawbacks greatly restrict the number of cells processable at once, making it difficult to achieve optimal cell density in each centrifugation bucket for large scale manufacturing. While large scale techniques, such as continuous flow centrifugation have been used to separate cell types from each other, these techniques have not been used to displace nuclei from nucleated cells to produce enucleated cells.

To overcome these drawbacks, the inventors of the present disclosure developed methods for cell processing comprising enucleating cells using continuous flow centrifugation. A continuous flow centrifugation saves processing time, where large volumes of material can be centrifuged at high centrifugal forces without the tedium of filling and decanting a lot of centrifuge tubes, or frequently starting and stopping the rotor. This combination of high centrifugal force and high throughput makes continuous flow processing useful for the large-scale of enucleated cells for biomedical application.

In addition, quality control of enucleated cell platforms for biomedical applications is a challenge, which become magnified by large scale manufacturing. Some of the many benefits of the enucleated cells disclosed herein are attributed to an absence of a nucleus, such as unwanted gene transfer in vivo, limited lifespan in vivo, and so forth. However, existing large scale manufacturing techniques result in a portion of nucleated parent cells in the resulting therapeutic composition, obviating the benefits of the enucleated cell platform.

To improve quality control, the inventors of the present disclosure engineered nucleated parent cells (from which the enucleated cells are derived) with biomolecular “suicide switches” that function to kill the nucleated parent cell when expression or activity of the suicide switch is induced. Employing biomolecular suicide switches, as disclosed herein, is a failsafe to maximize the enucleated cell fraction in the resulting composition. In some embodiments, a heterologous polynucleotide encoding the biomolecular suicide switch under the control of an inducible promoter is introduced into the nucleated parent cell using suitable techniques, such as transfection or transduction. After enucleation, if there are any nucleated cells left, the inducible promoter may be activated to express the biomolecular suicide switch and induce cell death. Without being bound by any particular theory, deployment of biomolecular “suicide switches” as a quality control measure, as disclosed herein, is suitable for virtually any cellular therapeutic or cell-based therapeutic delivery platform in which nucleated cells are unwanted. Such cellular therapeutic include, but are not limited to, Tumor-Infiltrating Lymphocyte (TIL) therapy, engineered T Cell Receptor (TCR) therapy, Chimeric Antigen Receptor (CAR) T cell therapy, Natural Killer (NK) cell therapy, and others. Such cell-mediated therapeutic delivery platforms include, but are not limited to, red blood cells, platelets, stems cells, leukocytes, and others disclosed in, for example Yu H, et al. Cell-mediated targeting drugs delivery systems. Drug Deliv. 2020 December; 27(1):1425-1437, which is hereby incorporated by reference in its entirety. Such techniques for employing biomolecular suicide switches can result in increased enucleation efficiencies as compared with mechanical enucleation techniques that do not utilize programed cell death. In some embodiments disclosed herein, the enucleation efficiency is increased by 5%, 10%, 15%, 20% 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 100%, 125%, 150%, 175%, or 200% or more.

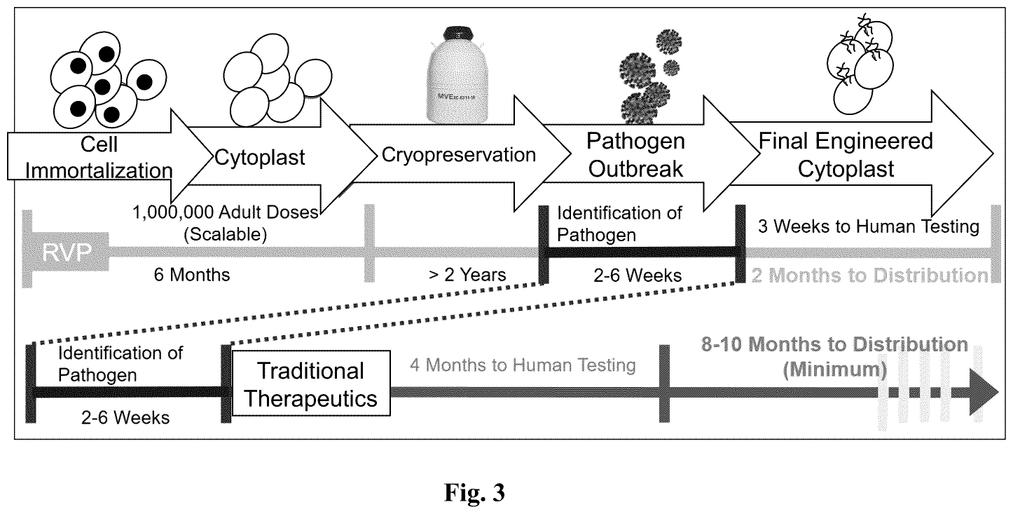

In addition to the advances in manufacturing scalability and quality control, the enucleated cell platform, itself, described herein possess certain advantages over existing cell-based therapeutic platforms that make it uniquely suitable for large scale use as therapeutic compositions. The enucleated cells described herein may be found in United States Patent Application No. 10,927,349, which is hereby incorporated by reference in its entirety. In addition, additional utility and advantages of the enucleated cells disclosed herein are discussed in International Application No. PCT/US2022/018007, filed Feb. 25, 2022, and published as WO/20221/83057 A1; and U.S. patent application Ser. No. 17/885,867, filed Aug. 11, 2022, and published in WO/20211/63222 A1, each of which is hereby incorporated by reference in its entirety. For example, there are certain therapeutic applications of cellular delivery platforms, such as in response to a pathogen outbreak, for which existing manufacturing timelines can limit scalability and speed necessary to address a public health emergency due to a pathogen outbreak. Existing therapeutic cellular therapies requiring extensive engineering take on the order of 12 months to develop at minimum. Whereas the enucleated cells disclosed herein can be extensively engineered before and after enucleation (e.g., with targeting moieties specific to target tissue, immune-system evading moieties to reduce phagocytosis in vivo, etc.), and then stored by suitable means disclosed here (e.g., lyophilization, cryohibemation, cryopreservation) for extended periods of time without sacrificing viability once revived. When a new pathogen or new strain of a known pathogen is identified, the biological activity of the enucleated cells (already engineered to express the appropriate targeting moieties, immune-system evading moieties, immune activators, etc.) can be restored (e.g., rehydration, thawing, etc.) and further engineered to express or carry a therapeutic agent for the prophylaxis or treatment of an infection by that recently discovered pathogen or strain. These benefits can be seen in FIG. 3, which illustrates that the process of manufacturing the enucleated cells of the present disclosure is roughly 2 months, as compared with suitable timelines, which is 12 months or longer.

Existing red blood cell or platelet therapeutic platforms are enucleated by erythropoiesis in which the blood cell is terminally differenced and intracellular organelles and ribosomes are eliminated, some of which are responsible for protein synthesis and secretion. Thus, the resulting red blood cell or platelet loses the cell-like functionality (e.g., protein expression, secretion, cell motility, chemokine sensing, homing capabilities, etc.) after enucleation by erythropoiesis that may be important for therapeutic applications, such as producing, delivering or secreting a therapeutic agent in vivo. By contrast, the enucleated cells described herein retain one or more intracellular organelles after enucleation that are endogenous to the parent cell. In some embodiments, all of the one or more intracellular organelles are retained. In some embodiments, fewer than all of the one or more intracellular organelles are retained. In some embodiments, the Golgi apparatus and/or the endoplasmic reticulum are retained, which are involved in protein synthesis and secretion. Retention of the one or more intracellular organelles at least partially enables the enucleated cells to synthesize or release the biomolecule disclosed herein (e.g., single-domain antibody, or portion thereof, targeting moiety, immune-evading moiety, etc.) in the absence of the nucleus.

The enucleated cells disclosed herein may be derived from virtually any nucleated cell (referred to herein as “parent” cell). In some embodiments, the parent cell is an immune cell. In some embodiments, the immune cell is a neutrophil, eosinophil, basophil, mast cell, monocyte, macrophage, dendritic cell, natural killer cell, or lymphocyte (B cells and T cells). In some embodiments, the parent cell is a stem cell. In some embodiments, the parent cell is an adult stem cell. In some embodiments, the parent cell is a mesenchymal stromal cell (MSC). In some embodiments, the enucleated cell is derived from an inducible pluripotent stem cell (iPSC). In some embodiments, the parent cell is not an erythrocyte. In some embodiments, the parent cell is not an erythroid precursor cell. In some embodiments, the parent cell is not an endothelial cell. In some embodiments, the parent cell is not an endothelial precursor cell.

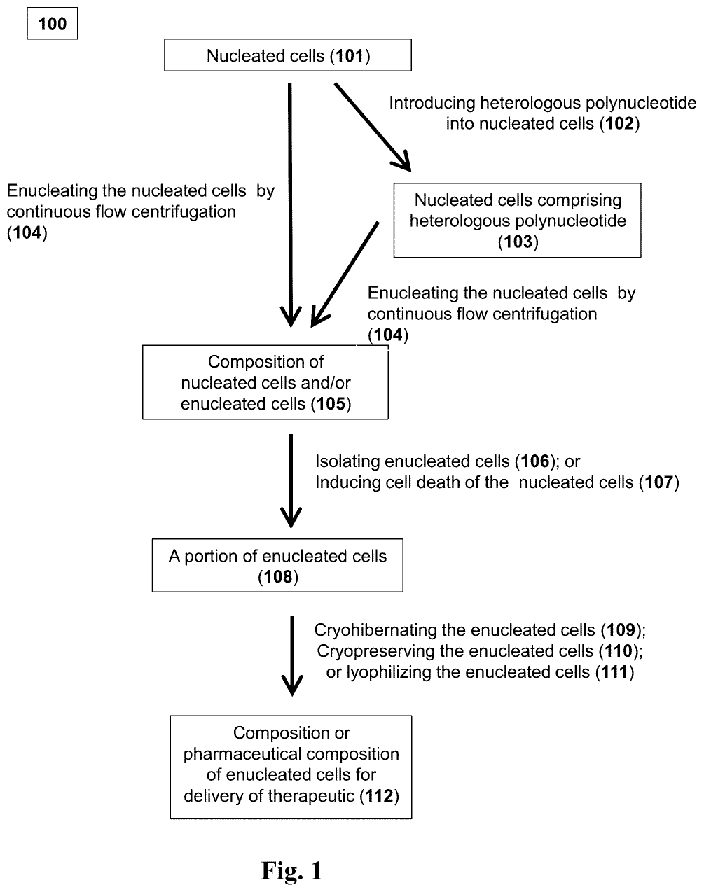

Described herein are methods for manufacturing enucleated cells in an increased quantity and purity, where the manufactured enucleated cells can be formulated into a composition or a pharmaceutical composition for treating a disease or condition in a subject in need thereof. FIG. 1 illustrates a non-limiting example of the manufacturing of the enucleated cells described herein (100). Nucleated cells (101) can be isolated from the subject and cultured in vitro for clonal expansion. In some embodiments, the nucleated cells (101) can also be immortalized or derived from a cell line. In some embodiments, the nucleated cells can be engineered (103) to comprise a heterologous polynucleotide (102), where the heterologous polynucleotide encodes a suicide moiety (e.g., a suicide gene) to kill the nucleated cells when needed. The nucleated cells can then be enucleated by continuous flow centrifugation (104). The use of continuous flow centrifugation for enucleating cells presents an improvement over the currently available methods for enucleation, where the enucleation conducted via continuous flow centrifugation increases the quantity (e.g., yield) or purity of the enucleated cells obtained from the nucleated cells. After obtaining the composition (105) of the enucleated cells (which may have residual nucleated cells), the composition can be further purified for the enucleated cells by selecting for markers of the enucleated cells (106) or by inducing cell death of the remaining residual nucleated cells (107) to obtain a portion of enucleated cells (108). The portion of enucleated cells can be cryohibernated (109), cryopreserved (110), lyophilized (111), or a combination thereof and be formulated into a composition or a pharmaceutical composition for delivery of therapeutic for treating the disease or condition in the subject.

In some embodiments, the methods of enucleation disclosed herein result in a composition comprising the enucleated cells (also referred to herein as “enucleated cell fraction” of the composition). In some embodiments, the composition further comprises less than or equal to about one (1) percent (%) residual nucleated cells (also referred to herein as “nucleated cell fraction” of the composition) by volume that were not enucleated. In some embodiments, the nucleated cell fraction comprises less than or equal to about 0.1%, 0.2%, 0.3%, 0.4%, 0.5%, 0.6%, 0.7%, 0.8%, or 0.9% of the composition by volume. In some embodiments, the nucleated cell fraction comprises 0.1% to about 0.2%, about 0.1% to about 0.3%, about 0.1% to about 0.4%, about 0.1% to about 0.5%, about 0.10% to about 0.6%, about 0.10% to about 0.7%, about 0.1% to about 0.8%, about 0.1% to about 0.9%, or about 0.1% to about 1.0% of the composition by volume. In some embodiments, the nucleated cell fraction comprises about 0.2% to about 0.3%, about 0.2% to about 0.4%, about 0.2% to about 0.5%, about 0.2% to about 0.6%, about 0.2% to about 0.7%, about 0.2% to about 0.8%, about 0.2% to about 0.9%, or about 0.2% to about 1.0% of the composition by volume. In some embodiments, the nucleated cell fraction comprises about 0.3% to about 0.4%, about 0.3% to about 0.5%, about 0.3% to about 0.6%, about 0.3% to about 0.7%, about 0.3% to about 0.8%, about 0.3% to about 0.9%, or about 0.3% to about 1.0% of the composition by volume. In some embodiments, the nucleated cell fraction comprises about 0.4% to about 0.5%, about 0.4% to about 0.6%, about 0.4% to about 0.7%, about 0.4% to about 0.8%, about 0.4% to about 0.9%, or about 0.4% to about 1.0% of the composition by volume. In some embodiments, the nucleated cell fraction comprises about 0.5% to about 0.6%, about 0.5% to about 0.7%, about 0.5% to about 0.8%, about 0.5% to about 0.9%, or about 0.5% to about 1.0% of the composition by volume. In some embodiments, the nucleated cell fraction comprises about 0.6% to about 0.7%, about 0.6% to about 0.8%, about 0.6% to about 0.9%, or about 0.6% to about 1.0% of the composition by volume. In some embodiments, the nucleated cell fraction comprises about 0.7% to about 0.8%, about 0.7% to about 0.9%, or about 0.7% to about 1.0% of the composition by volume. In some embodiments, the nucleated cell fraction comprises about 0.8% to about 0.9%, or about 0.8% to about 1.0% of the composition by volume. In some embodiments, the nucleated cell fraction comprises about 0.9% to about 1.0% of the composition by volume. In some embodiments, the nucleated cell fraction is eliminated by induced cell death following the enucleation. In some embodiments, the induced cell death is employed using biomolecular suicide switches that are expressed in response to an external stimulus, such as for example, exposure to a small molecule drug (e.g., rimiducid), a prodrug (e.g., ganciclovir), or the like.

Also described herein are pharmaceutical compositions and formulations comprising the compositions described herein, and a pharmaceutically acceptable: carrier, excipient, diluent, or nebulized inhalant. The pharmaceutical compositions are provided in pharmaceutical formulations. In some embodiments, the pharmaceutical formulations are formulated for administration to a subject as a combination therapy (e.g., prodrug, adjuvant, additional therapeutic agent, or other therapy) or monotherapy. In some embodiments, the pharmaceutical formulations are formulated for systemic administration or at the site of action, such as intratumoral administration.

Disclosed herein are kits comprising the composition disclosed herein and packaging material configured to deliver the composition to an individual. The kits disclosed herein may comprise a composition comprising a enucleated cell fraction and less than 0.10% nucleated cell fraction. In some embodiments, the kits further comprise instructions for further engineering the enucleated cells in the enucleated cell fraction, such as for example, to produce or secrete a therapeutic agent disclosed herein. In some embodiments, the kits further comprise a stimulus used to trigger expression or activity of biomolecular suicide switch in the nucleated cell fraction of the composition. In either case, the instructions may further comprise instructions for how to formulate the resulting composition into a pharmaceutical formulation for administration to a subject disclosed herein.

### Compositions

Disclosed herein are compositions and formulations thereof comprising enucleated cells capable of being extensively engineered to express an active agent, or portion thereof, in the absence of a nucleus. Such enucleated cells are viable cell-like entities capable of synthesizing, releasing (e.g., secreting), or delivering the active agent to a target cell or tissue in the absence of the nucleus. The compositions disclosed herein can be stored in a suspended biological stage by means such as cryohibernation, cryopreservation, or lyophilization for any period of time without impacting the viability of the enucleated cell once the biological activity is revived. Moreover, the compositions disclosed herein comprise less than or equal to about 0.10% of nucleated cells (e.g., parent cells that were not enucleated during the enucleation process), rendering the compositions disclosed herein optimal for therapeutic applications. The enucleated cells (as referred to here as “cytoplasts”) may further comprise naturally occurring cell-surface molecules retained from the parent cell. In some embodiments, the enucleated cells further comprise exogenous molecules, such as a targeting moiety, a transmembrane moiety, an additional therapeutic agent (e.g., other than the active agent) such as those disclosed herein.

### Enucleated Cell

The enucleated cells of the present disclosure are obtained or derived from a corresponding nucleated cell (referred to herein as a “parent cell”). The parent cell may be derived from a variety of different cell types, including eukaryotic cells. For example, an enucleated cell may be derived from an adult stem cell, a mesenchymal stromal cell (MSC), a natural killer (NK) cell, a macrophage, a myoblast, a neutrophil, endothelial cell, endothelial precursor cell, and/or a fibroblast. In some embodiments, an enucleated cell is derived from a mesenchymal stromal cell. In some embodiments, the enucleated cell is derived from an inducible pluripotent stem cell (iPSC). In some embodiments, the parent cell is derived from a cell is immortalized using suitable methods. In some embodiments, the enucleated cell comprises or retains one or more structural features of the parent cell, including intracellular organelles, one or more tunneling nanotubes, or a combination thereof. In some embodiments, the enucleated cell comprises one or more intracellular organelles for synthesis or secretion of an exogenous polypeptide (e.g., therapeutic agent) in absence of the nucleus. In some embodiments, the one or more intracellular organelles comprise a Golgi apparatus, an endoplasmic reticulum, or a combination thereof. In some embodiments, the enucleated cell comprises or expresses any one of the therapeutic agents described herein.

In some embodiments, the cell can originate from any organism having one or more cells. Non-limiting examples of cells include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell). In some embodiments, the cell is a somatic cell. In some embodiments, the cell is a stem cell or a progenitor cell. In some embodiments, the cell is a mesenchymal stem or progenitor cell. In some embodiments, the cell is a hematopoietic stem or progenitor cell. In some embodiments, the cell is a muscle cell, a skin cell, a blood cell, or an immune cell. Other non-limiting example of cells includes lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

In some embodiments, the cell is a eukaryotic cell. Non-limiting examples of eukaryotic cells include mammalian (e.g., rodent, non-human primate, or human), non-mammalian animal (e.g., fish, bird, reptile, or amphibian), invertebrate, insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. In some embodiments, the nucleated cell is a primary cell. In some embodiments, the nucleated cell is an immune cell (e.g., a lymphocyte (e.g., a T cell, a B cell), a macrophage, a natural killer cell, a neutrophil, a mast cell, a basophil, a dendritic cell, a monocyte, a myeloid-derived suppressor cell, an eosinophil). In some embodiments, the nucleated cell is a phagocyte or a leukocyte. In some embodiments, the nucleated cell is a stem cell (e.g., an adult stem cell (e.g., a hematopoietic stem cell, a mammary stem cell, an intestinal stem cell, mesenchymal stem cell, an endothelial stem cell, a neural stem cell, an olfactory adult stem cell, a neural crest stem cell, a testicular cell), an embryonic stem cell, an inducible pluripotent stem cell (iPS)). In some embodiments, the nucleated cell is a progenitor cell. In some embodiments, the nucleated cell is from a cell line. In some embodiments, the nucleated cell is a suspension cell. In some embodiments, the nucleated cell is an adherent cell. In some embodiments, the nucleated cell is a cell that has been immortalized by expression of an oncogene. In some embodiments, the nucleated cell is immortalized by the expression of human telomerase reverse transcriptase (hTERT) or any oncogene. In some embodiments, the nucleated cell is a patient or subject derived cell (e.g., an autologous patient-derived cell, or an allogenic patient-derived cell). In some embodiments, the nucleated cell is transfected with a vector (e.g., a viral vector (e.g., a retrovirus vector (e.g., a lentivirus vector), an adeno-associated virus (AAV) vector, a vesicular virus vector (e.g., vesicular stomatitis virus (VSV) vector), or a hybrid virus vector), a plasmid) before the nucleated cell is enucleated using any of the enucleation techniques described herein and known in the art.

In some embodiments, the cytoplast is derived from a cell autologous to the subject. In some embodiments, the cytoplast is derived from a cell allogenic to the subject.

In some embodiments, the cytoplast is derived from an immune cell. In some embodiments, the cytoplast is derived from a natural killer (NK) cell, a neutrophil, a macrophage, a lymphocyte, a fibroblast, an adult stem cell (e.g., hematopoietic stem cell, a mammary stem cell, an intestinal stem cell, a mesenchymal stem cell, a mesenchymal stromal cell, an endothelial stem cell, a neural stem cell, an olfactory adult stem cell, a neural crest stem cell, a skin stem cell, or a testicular cell), a mast cell, a basophil, an eosinophil, an endothelial cell, an endothelial cell precursor cell, or an inducible pluripotent stem cell.

In some embodiments, the parent cell may be enucleated and engineered for therapeutic use. In some embodiments, a parent cell may be treated with cytochalasin to soften the cortical actin cytoskeleton. In some embodiments, the nucleus is then physically extracted from the cell body by high-speed centrifugation in gradients of polysaccharide to generate an enucleated cell. In some embodiments, the polysaccharide is Ficoll for generating Ficoll gradients to generate an enucleated cell. Because enucleate cells and intact nucleated cells sediment to different layers in the Ficoll gradient, enucleated cells may be isolated and prepared for therapeutic purposes or fusion to other cells (nucleated or enucleated). The enucleation process can be clinically scalable to process tens of millions of cells by utilizing the methods described herein. In some embodiments, enucleated cells may be used as a disease-homing vehicle to deliver clinically relevant cargos or payloads to treat various diseases or conditions described herein.

In some embodiments, the enucleated cell comprises at least one therapeutic agent. In some embodiments, the enucleated cells disclosed herein express the therapeutic agent with one or more intracellular organelles in the absence of the nucleus. In some embodiments, the therapeutic agent is exogenous to the enucleated cell or parent (nucleated) cell thereof. In some embodiments, the enucleated cell expresses the therapeutic agent at the surface of the enucleated cell. In some embodiments, the therapeutic agent is secreted by the enucleated cell into extracellular space at a target tissue (e.g., a microenvironment). In some embodiments, the therapeutic agent is cargo (e.g., encapsulated by the enucleated cell) of the enucleated cell.

In some embodiments, the enucleated cell is obtained from a first subset of a plurality of nucleated cells. In some embodiments, the enucleated cells are in a composition, which further comprises a second subset of the plurality of the nucleated cells. In some embodiments, the second subset of the nucleated cells comprises less than about 0.1% by volume of the composition. In some embodiments, the second subset of the nucleated cells comprises less than about 0.5% by volume of the composition. In some embodiments, the second subset of the nucleated cells comprises less than about 1% by volume of the composition. In some embodiments, the second subset of the nucleated cells comprises less than about 5% by volume of the composition. In some embodiments, the second subset of the nucleated cells comprises less than about 10% by volume of the composition. In some embodiments, the second subset of the nucleated cells comprises less than about 15% by volume of the composition. In some embodiments, the second subset of the nucleated cells comprises less than about 20% by volume of the composition. In some embodiments, the second subset of the nucleated cells comprises less than about 25% by volume of the composition. In some embodiments, the second subset of the nucleated cells comprises less than about 30% by volume of the composition. In some embodiments, the second subset of the nucleated cells comprises less than about 40% by volume of the composition. In some embodiments, the second subset of the nucleated cells comprises less than about 50% by volume of the composition.

In one aspect, the nucleated cell (e.g., the parent cell prior to enucleation to yield the enucleated cell described herein) comprises a heterologous polynucleotide encoding a heterologous gene product configured to induce cell death of the nucleated cell. In some embodiments, the heterologous polynucleotide comprises a promoter. In some embodiments, the promoter is configured to activate transcription of the heterologous polynucleotide under conditions sufficient to express the heterologous gene product. In some embodiments, the promoter comprises an inducible promoter. In some embodiments, an inducible promoter is configured to activate transcription of the heterologous polynucleotide under conditions sufficient to express the heterologous gene product when induced.

In some embodiments, the enucleated cell described herein can be cryopreserved, cryohibernated, lyophilized, or a combination thereof. In some embodiments, the cryopreserved enucleated cell, following thawing, the enucleated cell is as viable as an otherwise comparable enucleated cell that is not cryopreserved. In some embodiments, the lyophilized enucleated cell is as viable as an otherwise comparable enucleated cell that is not lyophilized. In some embodiments, the cryohibemated enucleated cell is as viable as an otherwise comparable enucleated cell that is not cryohibernated.

In some embodiments, the enucleated cell or the composition comprising the enucleated cell may be cryopreserved (e.g., storing the enucleated cell or the composition comprising the enucleated cell at freezing temperature) or cryohibemated (e.g., storing the enucleated cell or the composition comprising the enucleated cell at a temperature that is between the ambient temperature and freezing temperature). The duration of cryopreservation or cryohibernation may be greater than or equal to about one hour, two hours, six hours, 12 hours, one day, two days, three days, four days, five days, six days, one week, two weeks, three weeks, four weeks, one month, two months, three months, or longer period of time. In some embodiments, the enucleated cell exhibits a viability after cryopreservation or cryohibernation that is greater than or equal to about 50%, 55%, 60%, 65%, 70%, 75%, 80%, 85%, 90%, 95%, or 99% similar to a comparable cell (e.g., a parent cell or an enucleated cell described herein that has not been cryopreserved or cryo-hibemated) after same the period of time of cryopreservation or cryohibernation. In some embodiments, the enucleated cell exhibits the viability following the cryohibernation as measured at 24 hours following the cryohibernation that is equal to or greater than the viability of a comparable enucleated cell that is not cryohibemated. In some embodiments, the enucleated cell exhibits the viability following the cryopreservation as measured at 24 hours following the cryopreservation that is equal to or greater than the viability of a comparable enucleated cell that is not cryopreserved. Viability in this context may be measured by Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, the viability is measured using Annexin-V cell surface staining. In some embodiments, the viability is measured by expression of the exogenous polypeptide. For example, the viability of the enucleated cell can be determined by the expression of the exogenous antibody or single-domain antibody expressed by the enucleated cell. In some embodiments, the viability is measured by expression of cell surface markers of any one of the cell surface markers described herein such as CD105, CD90, CD45, CXCR4, PSGL-1, or CCR2. In some embodiments, the viability is measured by the cell activity of the enucleated cell. In some embodiments, the viability is measured by the homing capability of the enucleated cell as determined by the chemosensing or chemokine homing activity described herein.

In some embodiments, the enucleated cell or the composition comprising the enucleated cell may be lyophilized. In some embodiments, the enucleated cell exhibits a viability after being reconstituted from lyophilization that is greater than or equal to about 50%, 55%, 60%, 65%, 70%, 75%, 80%, 85%, 90%, 95%, or 99% similar to a comparable cell (e.g., a parent cell or an enucleated cell described herein that has not been lyophilized).

In some embodiments, the enucleated cell or the composition comprising the enucleated cell may be dehydrated. In some embodiments, the enucleated cell exhibits a viability after being rehydrated from lyophilization that is greater than or equal to about 50%, 55%, 60%, 65%, 70%, 75%, 80%, 85%, 90%, 95%, or 99% similar to a comparable cell (e.g., a parent cell or an enucleated cell described herein that has not been dehydrated).

In some embodiments, the enucleated cell or the composition comprising the enucleated cell is stable at 4° C. for greater than or equal to about one hour, two hours, six hours, 12 hours, one day, two days, three days, four days, five days, six days, one week, two weeks, three weeks, four weeks, one month, two months, three months, or longer period of time. In some embodiments, the composition is stable at room temperature for greater than or equal to about one hour, two hours, six hours, 12 hours, one day, two days, three days, four days, five days, six days, one week, two weeks, three weeks, four weeks, one month, two months, three months, or longer period of time. In some embodiments, the composition is stable at 37° C. for greater than or equal to about one hour, two hours, six hours, 12 hours, one day, two days, three days, four days, five days, six days, one week, two weeks, three weeks, four weeks, one month, two months, three months, or longer period of time. In some embodiments, the enucleated cell or the composition comprising the enucleated cell may remain viable after being administered to a subject in need thereof for treating the disease or condition described herein. In some embodiments, the enucleated cell or the composition comprising the enucleated cell may remain viable after being administered to the subject for greater than or equal to about one hour, two hours, six hours, 12 hours, one day, two days, three days, four days, five days, six days, one week, two weeks, three weeks, four weeks, one month, two months, three months, or longer period of time.

In some embodiments, the enucleated cell may be obtained from a parent cell that is autologous to the subject, who is in need of the treatment by the enucleated cell described herein. In some embodiments, the enucleated cell may be obtained from a parent cell that is allogenic to the subject, who is in need of the treatment by the enucleated cell described herein.

Enucleated cells may be smaller than their nucleated counterparts (e.g. the nucleated parent cells), and for this reason may migrate better through small openings in the vasculature and tissue parenchyma. In addition, removing the large dense nucleus alleviates a major physical barrier allowing the cell to move freely through small openings in the vessels and tissue parenchyma. Therefore, enucleated cells have improved bio-distribution in the body and movement into target tissues. In some embodiments, an enucleated cell comprises at least 1 μm in diameter. In some embodiments, an enucleated cell is greater than 1 μm in diameter. In some embodiments, an enucleated cell is 1-100 m in diameter (e.g., 1-90 m, 1-80 m, 1-70 m, 1-60 μm, 1-50 μm, 1-40 μm, 1-30 μm, 1-20 μm, 1-10 μm, 1-5 μm, 5-90 μm, 5-80 μm, 5-70 μm, 5-60 μm, 5-50 μm, 5-40 μm, 5-30 μm, 5-20 μm, 5-10 μm, 10-90 μm, 10-80 μm, 10-70 μm, 10-60 μm, 10-50 μm, 10-40 μm, 10-30 μm, 10-20 μm, 10-15 μm 15-90 μm, 15-80 μm, 15-70 μm, 15-60 μm, 15-50 μm, 15-40 μm, 15-30 μm, 15-20 μm). In some embodiments, an enucleated cell is 10-30 μm in diameter. In some embodiments, the diameter of an enucleated cell is between 5-25 μm (e.g., 5-20 μm, 5-15 μm, 5-10 μm, 10-25 μm, 10-20 μm, 10-15 μm, 15-25 μm, 15-20 μm, or 20-m). In some embodiments, the enucleated cell has a diameter that is about 8 μm. In some embodiments, some enucleated cells may advantageously be small enough to allow for better homing or delivery to a target site. For examples, the enucleated cells described herein may pass through passages in narrow lung tissues or lung structures such as alveolar duct or microcapillary that most cells such as the parent cells may not pass through.

In some embodiments, the enucleated cell of the enucleated cell fraction has a diameter comprising less than or equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of an average diameter of a nucleated parent cell. In some embodiments, the enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, the enucleated cell of the enucleated cell fraction has a diameter comprising less than or equal to about 50% of an average diameter of the nucleated cells. In some embodiments, the enucleated cell of the enucleated cell fraction has a diameter comprising less than or equal to about 60% of an average diameter of the nucleated cells. In some embodiments, the enucleated cell of the enucleated cell fraction has a diameter comprising less than or equal to about 70% of an average diameter of the nucleated cells. In some embodiments, the enucleated cell of the enucleated cell fraction has a diameter comprising less than or equal to about 80% of an average diameter of the nucleated cells. In some embodiments, the enucleated cell of the enucleated cell fraction has a diameter comprising less than or equal to about 90% of an average diameter of the nucleated cells.

In some embodiments, enucleated cells possess significant therapeutic value, because they remain viable, do not differentiate into other cell types, secrete bioactive molecules, and may physically migrate/home for fewer than or equal to about 5 days, may be extensively enucleated ex vivo to perform specific therapeutic functions, and may be fused to the same or other cell types to transfer desirable production, natural or enucleated. Therefore, enucleated cells have wide utility as a cellular vehicle to deliver therapeutically important biomolecules and disease-targeting cargos including genes, viruses, bacteria, mRNAs, shRNAs, siRNA, polypeptides (including antibodies and antigen binding fragments), plasmids, gene-editing machinery, or nanoparticles. The present disclosure enables the generation of safe (e.g., no unwanted DNA is transferred to the subject), and controllable (e.g., cell death occurs in approximately 3-4 days) cell-based carrier that may be genetically enucleated to deliver specific disease-fighting and health promoting cargos to humans. In some embodiments, the enucleated cell remains viable and retain the function to migrate or home for greater than or equal to about 12 hours, 24 hours, 36 hours, 48 hours, 60 hours, 72 hours, 84 hours, 96 hours, 108 hours, 5 days, 6 days, 7 days, 8 days, 9 days, or longer after being administered to the subject in need thereof.

In some embodiments, the enucleated cell is engineered to express at least one of an exogenous DNA molecule, an exogenous RNA molecule, an exogenous protein, or an exogenous protein, gene-editing machinery or combinations thereof. In some embodiments, the exogenous DNA molecule is a single-stranded DNA, a double-stranded DNA, an oligonucleotide, a plasmid, a bacterial DNA molecule, a DNA virus, or combinations thereof. In some embodiments, the exogenous RNA molecule is messenger RNA (mRNA), small interfering RNA (siRNA), microRNA (miRNA), short hairpin RNA (shRNA), an RNA virus, or combinations thereof. In some embodiments, the exogenous protein is a cytokine, a growth factor, a hormone, an antibody or the antigen-binding fragment thereof, an enzyme, or combinations thereof. In some embodiments, the antibody is a single-domain antibody or antigen-binding fragment thereof. In some embodiments, parental cells (e.g., nucleated cells) are genetically enucleated before enucleation (e.g., pre-enucleation). In some embodiments, the parent cell is genetically enucleated after enucleation (e.g., post-enucleation).

### Transmembrane Moiety

Described herein, in some aspects, are enucleated cells or compositions comprising the enucleated cell comprising at least one transmembrane moiety. In some embodiments, the enucleated cell comprises an exogenous polypeptide. The exogenous polypeptide may be covalently fused to a transmembrane moiety. In some embodiments, the exogenous polypeptide is complexed to the transmembrane moiety. In some embodiments, the transmembrane moiety comprises a full length protein or a variation thereof or a fragment thereof. In some embodiments, the transmembrane moiety is endogenous to the parent cell that is being enucleated for obtaining the enucleated cell. In some embodiments, the transmembrane moiety may be an exogenous transmembrane moiety to the parent cell or to the enucleated cell. In some embodiments, the transmembrane moiety is selected from a transmembrane protein comprising a single transmembrane α-helix (bitopic membrane protein). The transmembrane moiety comprises a polytopic transmembrane α-helical protein. In some embodiments, the transmembrane moiety comprises a polytopic transmembrane n-sheet protein. In some embodiments, the transmembrane moiety comprises a Type I, II, III, or IV transmembrane protein. Non-limiting examples of transmembrane protein may include CD4, CD14, glycophorin a (GPA), or any combination of integrins.

In some embodiments, the transmembrane moiety is added to the exogenous polypeptide by way of a modification. For example, a transmembrane moiety may be added to the N or C-terminus of the exogenous polypeptide to insert the exogenous polypeptide into the cell membrane of the enucleated cell described herein. Non-limiting examples of modifications that are made to the exogenous polypeptide to add the transmembrane moiety may include adding glycosylphosphatidylinositol, farnesyl, palmitate, myristate, or a combination thereof to the exogenous polypeptide.

In some embodiments, the transmembrane moiety is genetically modified to be fused or complexed with the at least one exogenous therapeutic agent described herein. In some embodiments, the transmembrane moiety is genetically modified to fuse to the at least one exogenous therapeutic agent described herein. In some embodiments, the enucleated cell comprises an immune-evading moiety. In some embodiments, the immune-evading comprises a “don't eat me” signaling peptide, such as CD47 (e.g., NCBI Gene ID 961), programmed cell death 1 ligand 1 (PD-L1,e.g., NCBI Gene ID 29126), major histocompatibility complex, class I, E (HLA-E, e.g., NCBI Gene ID 3133), major histocompatibility complex, class I, G (HLA-I, e.g., NCBI Gene ID 3135), a fragment thereof, or a combination thereof.

### Targeting Moiety

Described herein, in some aspects, are enucleated cells comprising a targeting moiety. The targeting moiety described herein is designed to guide the enucleated cell to a target cell or target environment (e.g., tissue) in a subject following delivery (e.g., systemic delivery) of the enucleated cell to the subject. In some embodiments, the targeting moiety is expressed on the surface of the enucleated cell. In some embodiments, the targeting moiety is complexed with a transmembrane moiety described herein. In some embodiments, the targeting moiety is secreted by the enucleated cell. In some embodiments, the enucleated cells comprising the targeting moiety localizes at the target cell or target environment with a 2-fold, 5-fold, 10-fold, 50-fold, 100-fold, 200-fold, 500-fold, 1,000-fold, 5,000-fold, or 10,000-fold increase as compared to localization of a comparable enucleated cell lacking the targeting moiety. In some embodiments, the enucleated cell comprising the targeting moiety localizes at the target cell or target environment with an increase of 50%, 55%, 60%, 65%, 70%, 75%, 80%, 85%, 90%, 95%, or 99% as compared with a comparable enucleated cell lacking the targeting moiety. In some embodiments, the target cell or target environment is in vivo. In some embodiments, the target cell or target environment is ex vivo.

In some embodiments, the targeting moiety comprises an exogenous antibody or an exogenous antigen-binding fragment for targeting a biomarker described herein. In some embodiments, the targeting moiety comprises an exogenous antibody or an exogenous antigen-binding fragment for targeting a chemokine receptor or a chemokine ligand, or portion thereof, involved in chemokine signaling. In some embodiments, the exogenous antibody is an exogenous single-domain antibody or fragment thereof.

In some embodiments, the targeting moiety targets the biomarker expressed by, or associated with, a target cell or with a microenvironment. In some embodiments, the biomarker may be released by the target cell. The biomarker may indicate the presence of the disease or the condition. In some embodiments, the biomarker is expressed by immune cells responding to the target cell or the microenvironment associated with the disease or the condition. In some embodiments, the biomarker may be an epitope or antigen. In some embodiments, the biomarker comprising the epitope may be bound by an antibody that is different from the antibody or the antigen-binding fragment thereof that confers therapeutic property (e.g., the therapeutic agent).

In some embodiments, the targeting moiety targets a biomarker expressed or released by a lung cell or a lung cancer cell. Non-limiting example of cancer cell biomarkers includes carbonic anhydrase 9 (CA9, e.g., NCBI Gene ID 768), carbonic anhydrase 12 (CA12, e.g., NCBI Gene ID 771), cancer/testis antigen 83 (CXorf61; e.g., NCBI Gene ID203413), desmoglein 3 (DSG3 (e.g., NCBI Gene ID 1830), FAT atypical cadherin 2 (FAT2 (e.g., NCBI Gene ID 2196), G protein-coupled receptor 87 (GPR87, e.g., NCBI Gene ID 53836), KISS1 receptor (KISSIR, e.g., NCBI Gene ID 84634), LY6/PLAUR domain containing 3 (LYPD3. e.g., NCBI Gene ID 27076), solute carrier family 7 member 11 (SLC7A11, e.g., NCBI Gene ID 23657), TMPRSS4 (e.g., NCBI Gene ID 56649), transmembrane serine protease 4 (TFPI, e.g., NCBI Gene ID 7035), midkine (MDK, e.g., NCBI Gene ID 4192), secreted phosphoprotein 1 (OPN, e.g., NCBI Gene ID 6696), matrix metallopeptidase 2 (MMP2, e.g., NCBI Gene ID 4313), TIMP metallopeptidase inhibitor 1 (TIMP1, e.g., NCBI Gene ID 7076), cell adhesion molecule 5 (CEA, e.g., NCBI Gene ID 1048), cytokeratin 19 fragment (CYFRA 21-1, e.g., NCBI Gene ID 3880), serpin family B member 3 (SCC, e.g., NCBI Gene ID 6317), advanced glycosylation end-product specific receptor (AGER, e.g., NCBI Gene ID 177), adipogenesis regulatory factor (C10orfl16, e.g., NCBI Gene ID 10974), adducin 2 (ADD2, e.g., NCBI Gene ID 119), periaxin (PRX, e.g., NCBI Gene ID 57716), laminin subunit beta 3 (LAMB3, e.g., NCBI Gene ID 3914), synemin (SYNM, e.g., NCBI Gene ID 23336), spectrin alpha, erythrocytic 1 (SPTA1, e.g., NCBI Gene ID 6708), ankyrin 1 (ANKI, e.g., NCBI Gene ID 286), hemoglobin subunit epsilon 1 (HBE1, e.g., NCBI Gene ID 3046), hemoglobin subunit gamma 1 (HBG1, e.g., NCBI Gene ID 3047), carbonic anhydrase 1 (CA1, e.g., NCBI Gene ID 759), tenascin XB (TNXB, e.g., NCBI Gene ID 7148), multimerin 2 (MMRN2, e.g., NCBI Gene ID 79812), hemoglobin subunit alpha 1 (HBA1, e.g., NCBI Gene ID 3039), caveolin 1 (CAV1, e.g., NCBI Gene ID 857), hemoglobin subunit beta (HBB, e.g., NCBI Gene ID 3043), collagen type VI alpha 6 chain (COL6A6, e.g., NCBI Gene ID 131873), chromosome 1 open reading frame 198 (Clorfl98, e.g., NCBI Gene ID 84886), chloride intracellular channel 2 (CLIC2, e.g., NCBI Gene ID 1193), transcriptional regulator of SdpC synthesis operon (ArsR family) (SDPR, e.g., NCBI Gene ID 8436), EH domain containing 2 (EHD2, e.g., NCBI Gene ID 30846), apolipoprotein A2 (APOA2, e.g., NCBI Gene ID 336), NADH: ubiquinone oxidoreductase subunit B7 (NDUFB7, e.g., NCBI Gene ID 4713), protein kinase C delta binding protein (PRKCDBP, e.g., NCBI Gene ID 112464), laminin subunit alpha 3 (LAMA3, e.g., NCBI Gene ID), EvC ciliary complex subunit 2 (LBN, e.g., NCBI Gene ID 132884), serpin family A member 3 (ACT, e.g., NCBI Gene ID 12), insulin like growth factor binding protein 3 (3 IGFBP3, e.g., NCBI Gene ID 3486), prostaglandin D2 synthase (L-PGDS, e.g., NCBI Gene ID 5730), retinoic acid receptor beta (HAP, e.g., NCBI Gene ID 5915), hepatocyte growth factor (HGF, e.g., NCBI Gene ID 3082), eukaryotic translation initiation factor 4 gamma 2 (AAG1/2, e.g., NCBI Gene ID 1982), clusterin (CLU, e.g., NCBI Gene ID 1191), streptococcal superantigen SSA (SSA, e.g., NCBI Gene ID 6737), tetanic (TTA, e.g., NCBI Gene ID 100189453), apolipoprotein A4 (APOA4, e.g., NCBI Gene ID 337), fibrinogen-like protein A (FIBA, e.g., NCBI Gene ID 105209070), serum amyloid A cluster (SAA, e.g., NCBI Gene ID 6288), ceruloplasmin (CP, e.g., NCBI Gene ID 1356), haptoglobin (HP, e.g., NCBI Gene ID 3240), transthyretin (TTR, e.g., NCBI Gene ID 7276), keratin 2 (KRT2A, e.g., NCBI Gene ID 3849), glutamate transporter (GLT1B, e.g., NCBI Gene ID 6506), casein kinase 1 (CK1, e.g., NCBI Gene ID 1452), AKT serine/threonine kinase 1 (AKT, e.g., NCBI Gene ID 207), mannose binding lectin 2 (MBL2, e.g., NCBI Gene ID 4153), fibrinogen alpha chain (FGA, e.g., NCBI Gene ID 2243), gelsolin (GSN, e.g., NCBI Gene ID 2934), haptoglobin (HP, e.g., NCBI Gene ID 3240), ficolin 3 (FCN3, e.g., NCBI Gene ID 8547), camosine dipeptidase 1 (CNDP1, e.g., NCBI Gene ID 84735), calcitonin related polypeptide alpha (CALCA, e.g., NCBI Gene ID 796), carbamoyl-phosphate synthase 1 (CPS1, e.g., NCBI Gene ID 1373), chromogranin B (CHGB, e.g., NCBI Gene ID 1114), involucrin (IVL, e.g., NCBI Gene ID 3713), anterior gradient 2 (AGR2, e.g., NCBI Gene ID 10551), nuclear autoantigenic sperm protein (NASP, e.g., NCBI Gene ID 4678), phosphofructokinase, platelet (PFKP, e.g., NCBI Gene ID 5214), thrombospondin 2 (THBS2, e.g., NCBI Gene ID 7058), thioredoxin domain containing 17 (TXNDC17, e.g., NCBI Gene ID 84817), proprotein convertase subtilisin/kexin type 1 (PCSK1, e.g., NCBI Gene ID 5122), cellular retinoic acid binding protein 2 (CRABP2, e.g., NCBI Gene ID 1382), acyl-CoA binding domain containing 3 (ACBD3, e.g., NCBI Gene ID 64746), desmoglein 2 (DSG2, e.g., NCBI Gene ID 1829), LPS responsive beige-like anchor protein (LRBA, e.g., NCBI Gene ID 987), serine/threonine kinase receptor associated protein (STRAP, e.g., NCBI Gene ID 11171), VGF nerve growth factor inducible (VGF, e.g., NCBI Gene ID 7425), NOP2 nucleolar protein (NOP2, e.g., NCBI Gene ID 4839), lipocalin 2 (LCN2, e.g., NCBI Gene ID 3934), creatine kinase, mitochondrial 1B (CKMT1B, e.g., NCBI Gene ID 1159), aldo-keto reductase family 1 member B10 (AKR1B10, e.g., NCBI Gene ID 57016), carboxypeptidase D (CPD, e.g., NCBI Gene ID 1362), proteasome activator subunit 3 (PSME3, e.g., NCBI Gene ID 10197), villin 1 (VIL1, e.g., NCBI Gene ID 7429), serpin family B member 5 (SERPINB5, e.g., NCBI Gene ID 5268), ribosomal protein L5 (RPL5, e.g., NCBI Gene ID 6125), plakophilin 1 (PKP1, e.g., NCBI Gene ID 5317), ribosomal protein L10 (RPL10, e.g., NCBI Gene ID 6134), aldo-keto reductase family 1 member B10 (AKR1B10, e.g., NCBI Gene ID 57016), aldo-keto reductase family 1 member C1 (AKR1C1, e.g., NCBI Gene ID 1645), proliferating cell nuclear antigen (PCNA, e.g., NCBI Gene ID 5111), ribosomal protein S2 (RPS2, e.g., NCBI Gene ID 6187), aldo-keto reductase family 1 member C3 (AKR1C3, e.g., NCBI Gene ID 8644), acyl-CoA binding domain containing 3 (ACBD3, e.g., NCBI Gene ID 64746), visinin like 1 (VSNL1, e.g., NCBI Gene ID 7447), adenosylhomocysteinase (AHCY, e.g., NCBI Gene ID 191), IMMP10, activated kinase 2 (PAK2, e.g., NCBI Gene ID 5062), involucrin (IVL, e.g., NCBI Gene ID 3713), isoleucine-tRNA synthetase (IARS, e.g., NCBI Gene ID 3376), proteasome 26S subunit ubiquitin receptor, non-ATPase 2 (PSMD2, e.g., NCBI Gene ID 5708), guanylate binding protein 5 (GBP5, e.g., NCBI Gene ID 115362), minichromosome maintenance complex component 6 (MCM6, e.g., NCBI Gene ID 4175), N-myc downstream regulated 1 (NDRG1, e.g., NCBI Gene ID 10397), NOP58 ribonucleoprotein (NOP58, e.g., NCBI Gene ID 51602), 5100 calcium binding protein A2 (S100A2, e.g., NCBI Gene ID 6273), neuregulin 1 (NRG1, e.g., NCBI Gene ID 3084), neuregulin 2 (NRG2, e.g., NCBI Gene ID 9542), carnosine dipeptidase 1 (CNDP1, e.g., NCBI Gene ID 84735), ubiquitin cross-reactive protein (UCRP, e.g., NCBI Gene ID 9636), crammer (CER, e.g., NCBI Gene ID 8110), plasminogen activator (UPA, e.g., NCBI Gene ID 5328), matrix metallopeptidase 14 (MT1-MMP, e.g., NCBI Gene ID 4323), stratifin (SFN, e.g., NCBI Gene ID 2810), transferrin (TF, e.g., NCBI Gene ID 7018), albumin (ALB, e.g., NCBI Gene ID 213), 5100 calcium binding protein A9 (S100A9, e.g., NCBI Gene ID 6280), stathmin 1 (STMN, e.g., NCBI Gene ID 3925), Enolase (ENO), plasminogen activator (PLAU, e.g., NCBI Gene ID 5328), insulin like growth factor binding protein 7 (IGFBP7, e.g., NCBI Gene ID 3490), matrix metallopeptidase 14 (MMP14, e.g., NCBI Gene ID 4323), thrombospondin 1 (THBS1, e.g., NCBI Gene ID 7057), or thrombospondin 2 (THBS2, e.g., NCBI Gene ID 7058).

In some embodiments, the targeting moiety targets a biomarker expressed or released by a cancer cell that has metastasized. For example, the cancer cell may arise from one tissue and subsequently metastasizes to a different location. In some embodiments, the metastasized cancer cell expresses the non-limiting example of cancer biomarker described herein. In some embodiments, the metastasized cancer cell expresses cancer biomarker includes Melanoma Associated Antigen (MAGE family member A3 (MAGE-A3, e.g., NCBI Gene ID 4102)), Membrane associated glycoprotein (MUC-1, e.g., NCBI Gene ID 4582), glycoproteine-epithelial cell adhesion molecule (EpCAM, e.g., NCBI Gene ID 4072), KRAS Proto-Oncogene (KRAS, e.g., NCBI Gene ID 3845), Anaplastic lymphoma kinase (ALK, e.g., NCBI Gene ID 238), Cytotoxic T-Lymphocyte Associated Protein 4 (CTLA-4, e.g., NCBI Gene ID 1493), Programmed cell death protein 1 (PD-1, e.g., NCBI Gene ID 5133), Epidermal growth factor (EGF, e.g., NCBI Gene ID 1950), Serine protease ester (EA, e.g., NCBI Gene ID 5328), Telomerase reverse transcriptaseh (TERT, e.g., NCBI Gene ID 7015), PRAME Nuclear Receptor Transcriptional Regulator (PRAME, e.g., NCBI Gene ID 23532), Receptor tyrosine-protein kinase erbB-2 (HER, e.g., NCBI Gene ID 2064), or Vascular endothelial growth factor (VEGF, e.g., NCBI Gene ID 7422), Carcinoembryonic antigen (CEA, e.g., NCBI Gene ID 1048), MAGE family member A1 (MAGE-A1, e.g., NCBI Gene ID 4100), MAGE family member A1 MAGE-A4, e.g., NCBI Gene ID 4103), Survivin, Six Transmembrane Epithelial Antigene of the Prostate 1 (STEAPI, e.g., NCBI Gene ID 26872), SRY (sex determining region Y)-box 2 (SOX2, e.g., NCBI Gene ID 6657), or Cancer/testis antigen 1 (CTAGlB, e.g., NCBI Gene ID 1485).

In some embodiments, the targeting moiety targets a biomarker expressed or released by an endothelial cell. In some embodiments, the endothelial cell is a blood vessel cell. In some embodiments, the endothelial cell is a lymphatic vessel cell. In some embodiments, the biomarker is expressed or released by a blood vessel cell. In some embodiments, the biomarker is expressed or released by a lymphatic vessel cell. Non-limiting examples of the endothelial cell biomarker include angiotensin I converting enzyme (ACE/CD143, e.g., NCBI Gene ID 1636), CD93 molecule (ClqR1/CD93, e.g., NCBI Gene ID 22918), cadherin 5 (VE-Cadherin, e.g., NCBI Gene ID 1003), D6 protein (CC Chemokine Receptor D6, e.g., NCBI Gene ID 1238), platelet and endothelial cell adhesion molecule 1 (CD31/PECAM-1, e.g., NCBI Gene ID 5175), CD34 molecule (CD34, e.g., NCBI Gene ID 947), CD36 molecule (CD36/SR-B3, e.g., NCBI Gene ID 948), CD151 molecule (CD151, e.g., NCBI Gene ID 977), CD160 molecule (CD160, e.g., NCBI Gene ID 11126),

CD300 molecule like family member g (CD300g/Nepmucin, e.g., NCBI Gene ID 146894), CDC like kinase 1 (CL-K1/COLEC11, e.g., NCBI Gene ID 78989), cleavage factor polyribonucleotide kinase subunit 1 (CL-P1/COLEC12, e.g., NCBI Gene ID 81035), Coagulation Factor III/Tissue Factor (e.g., NCBI Gene ID 2152), C-type lectin domain family 4 member M (DC-SIGNR/CD299, e.g., NCBI Gene ID 10332), discoidin, CUB and LCCL domain containing 2 (DCBLD2/ESDN, e.g., NCBI Gene ID 131566), endothelial cell surface expressed chemotaxis and apoptosis regulator (ECSCR, e.g., NCBI Gene ID 641700), basigin (Ok blood group) (EMMPRIN/CD147, e.g., NCBI Gene ID 682), Endoglin/CD105 (e.g., NCBI Gene ID 5077), Endomucin (e.g., NCBI Gene ID 2022), Endosialin/CD248 (e.g., NCBI Gene ID 57124), protein C receptor (EPCR, e.g., NCBI Gene ID 10544), Erythropoietin R (e.g., NCBI Gene ID 2056), endothelial cell adhesion molecule (ESAM, e.g., NCBI Gene ID 90952), fatty acid binding protein 5 (FABP5/E-FABP, e.g., NCBI Gene ID 2171), fatty acid binding protein 6 (FABP6, e.g., NCBI Gene ID 2172), intercellular adhesion molecule 1 (ICAM-1/CD54, e.g., NCBI Gene ID 3383), intercellular adhesion molecule 2 (ICAM-2/CD102, e.g., NCBI Gene ID 3384), interleukin 1 receptor (IL-1 RI, e.g., NCBI Gene ID 3553), Interleukin 13 receptor, alpha 1 (IL-13 R alpha 1, e.g., NCBI Gene ID 3597), Integrin alpha 4/CD49d (e.g., NCBI Gene ID 3676), Integrin alpha 4 beta 1 (e.g., NCBI Gene ID 3688), Integrin alpha 4 beta 7/LPAM-1 (e.g., NCBI Gene ID 3676), Integrin beta 2/CD18 (e.g., NCBI Gene ID 3689), KLF transcription factor 4 (KLF4, e.g., NCBI Gene ID 9314), lymphatic vessel endothelial hyaluronan receptor 1 (LYVE-1, e.g., NCBI Gene ID 10894), melanoma cell adhesion molecule (MCAM/CD146, e.g., NCBI Gene ID 4162), nectin cell adhesion molecule 2 (Nectin-2/CD112, e.g., NCBI Gene ID 5819), PD-ECGF/Thymidine Phosphorylase (e.g., NCBI Gene ID 1890), Podocalyxin (e.g., NCBI Gene ID 5420), Podoplanin (e.g., NCBI Gene ID 10630), sphingosine-1-phosphate receptor 1 (S1P1/EDG-1, e.g., NCBI Gene ID 1901), sphingosine-1-phosphate receptor 2 (S1P2/EDG-5, e.g., NCBI Gene ID 9294), sphingosine-1-phosphate receptor 3 (S1P3/EDG-3, e.g., NCBI Gene ID 1903), sphingosine-1-phosphate receptor 4 (S1P4/EDG-6, e.g., NCBI Gene ID 8698), sphingosine-1-phosphate receptor 5 (S1P5/EDG-8, e.g., NCBI Gene ID 53637), E-Selectin/CD62E (e.g., NCBI Gene ID 6401), P-Selectin/CD62P (e.g., NCBI Gene ID 6403), slow as molasses (SLAM/CD150, e.g., NCBI Gene ID 6504), Stabilin-1 (e.g., NCBI Gene ID 23166), Stabilin-2 (e.g., NCBI Gene ID 55576), plexin domain containing 1 (TEM7/PLXDC1, e.g., NCBI Gene ID 57125), ANTXR cell adhesion molecule 1 (TEM8/ANTXR1, e.g., NCBI Gene ID 84168), Thrombomodulin/BDCA-3 (e.g., NCBI Gene ID Thrombomodulin), thrombospondin type 1 domain containing 1 (THSD1, e.g., NCBI Gene ID 55901), thrombospondin type 1 domain containing 7A (THSD7A, e.g., NCBI Gene ID 221981), TEK receptor tyrosine kinase (Tie-2, e.g., NCBI Gene ID 7010), TNF receptor superfamily member 1A (TNF RI/TNFRSFlA, e.g., NCBI Gene ID 7132), TNF receptor superfamily member 1B (TNF RII/TNFRSFlB, e.g., NCBI Gene ID 7133), basigin (Ok blood group) (TRA-1-85/CD147, e.g., NCBI Gene ID 682), TNF receptor superfamily member 10b (TRAIL R2/TNFRSF10B, e.g., NCBI Gene ID 8795), TNF receptor superfamily member 10a (TRAILR1/TNFRSF10A, e.g., NCBI Gene ID 8797), vascular cell adhesion molecule 1 (VCAM-1/CD106, e.g., NCBI Gene ID 7412), EGF like domain multiple 7 (VE-Statin, e.g., NCBI Gene ID: 51162), fms related receptor tyrosine kinase 1 (VEGFR1/Flt-1, e.g., NCBI Gene ID 2321), kinase insert domain receptor (VEGFR2/KDR/Flk-1, e.g., NCBI Gene ID 3791), fins related receptor tyrosine kinase 4 (VEGFR3/Flt-4, e.g., NCBI Gene ID 2324), angiogenic factor with G-patch and FHA domains 1 (VGSQ, e.g., NCBI Gene ID 55109), or von Willebrand Factor domain 2 (vWF-A2, e.g., NCBI Gene ID 7450).

In some embodiments, the targeting moiety comprises a chemokine receptor or a chemokine ligand, or portion thereof, involved in chemokine signaling, such as for example, SDF-1α/CXCR4, CCL2/CCR2, or adhesion molecules, such as for example, PSGL-1. As shown herein, the enucleated cell may be enucleated to express functional CXCR4, CCR2 as well as glycosylated PSGL-1, which may greatly promote the specific targeting of the enucleated cell. In some embodiments, the targeting moiety, such as CXCR4, CCR2 or PSGL-1 may be expressed on the surface of the enucleated cell. Non-limiting examples of cell surface proteins that may be expressed on the cell surface of the enucleated cell as the targeting moiety include chemokines such as CXCR4, CCR2, CCR1, CCR5, CXCR7, CXCR2, and CXCR1. In some embodiments, the enucleated cell may be enucleated to secrete the targeting moiety or is tethered to the extracellular matrix, e.g., SDF1α or CCL2. Non-limiting examples of targeting moiety that may be secreted by the enucleated cell include SDF1α, CCL2, CCL3, CCL5, CCL8, CCL1, CXCL9, CXCL10, CCL11 and CXCL12. In some embodiments, the enucleated cell comprises cell-matrix receptors and cell-cell adhesion molecules include integrins, cadherins, glycoproteins, and heparin sulfate proteoglycans.

In some embodiments, the enucleated cells may further include (e.g. by engineering or from the cell from which they were obtained) a surface marker that aids in their evasion of the subject immune system. For example, in some embodiments, the enucleated cells may include a CD47, PD-L1, HLA-E, HLA-G, a fragment thereof, or a combination thereof. Without being bound by any particular theory, it is believed that a CD47, PD-L1, HLA-E, HLA-G, a fragment thereof, or a combination thereof helps to prevent the enucleated cells from being phagocytosed by macrophages. Non-limiting examples of cell-matrix receptors and cell-cell adhesion molecules include integrins, cadherins, glycoproteins, or heparin sulfate proteoglycans. In some embodiments, the cell-matrix receptors or cell-cell adhesion molecules include PD-L1, HLA-E, or HLA-G. Non-limiting examples of therapeutic molecules include tumor antigens and immunomodulatory peptides, polyamines, and ATP. In some embodiments, the therapeutic molecules can be recognized by immune cells and can induce immune response. For example, the therapeutic molecules can be 4-1BB or any one of the cytokines described herein to induce immune response.

### Therapeutic Agent

In some embodiments, the enucleated cell of the present disclosure comprises at least one therapeutic agent. In some embodiments, the enucleated cell of the present disclosure comprises at least two, three, four, five, six, seven, eight, nine, ten, or more therapeutic agents. In some embodiments, the therapeutic agent comprises an active agent. In some embodiments, the therapeutic agent is exogenous to the enucleated cell or parent cell thereof. An active agent comprises at least one of a DNA molecule, a RNA molecule, a protein (e.g., an enzyme, an antibody, an antigen, a toxin, cytokine, a protein hormone, a growth factor, a cell surface receptor, or a vaccine), a peptide (e.g., a peptide hormone or an antigen), a small molecule (e.g., a steroid, a polyketide, an alkaloid, a toxin, an antibiotic, an antiviral, a colchicine, a taxol, a mitomycin, or emtansine), a gene editing factor, a nanoparticle, or another active agent (e.g., bacteria, bacterial spores, bacteriophages, bacterial components, viruses (e.g., oncolytic viruses), exosomes, lipids, or ions). In some embodiments, an enucleated cell is engineered to produce (e.g., express, and in some cases, release or secrete) the therapeutic agent. In some embodiments, the parent may be engineered to produce the therapeutic agent prior to enucleation to produce the enucleated cell. Non-limiting examples of oncolytic viruses include Talimogene laherparepvec, Onyx-015, GL-ONC1, CV706, Voyager-V1, and HSV-1716. Some wild-type viruses also show oncolytic behavior, such as Vaccinia virus, Vesicular stomatitis virus, Poliovirus, Reovirus, Senecavirus, ECHO-7, and Semliki Forest virus.

The therapeutic agent may be, or include, a targeting moiety described herein. Non-limiting example of the targeting moieties that may be produced by or contained in an enucleated cell includes chemokine receptors, adhesion molecules, and antigens. In some embodiments, the therapeutic agent may be, or include, a transmembrane moiety described herein.

In some embodiments, the therapeutic agent is recombinantly expressed by the enucleated cell or parent cell thereof. In some embodiments, the parent cell from which the enucleated cell is derived or obtained is engineered to produce or express the therapeutic agent. In some embodiments, expression of the therapeutic agent is stable (e.g., permanent). In some embodiments, the expression of the therapeutic agent by the parent cell is transient (e.g., non-permanent). In some embodiments, the parent cell is enucleated prior to engineering the enucleated cell to recombinantly express the therapeutic agent.

In some embodiments, the therapeutic agent is not naturally expressed (e.g., in the absence of engineering) in the cell from which the enucleated cell was derived or obtained (e.g., the therapeutic agent is exogenous to the parent cell). In some embodiments, the therapeutic agent is not naturally expressed in the subject (e.g., the therapeutic agent is exogenous to the subject). In some embodiments, the therapeutic agent is not naturally expressed in the subject at the intended site of therapy (e.g., a tumor, or a particular tissue, such as the brain, the intestine, the lungs, the heart, the liver, the spleen, the pancreas, muscles, eyes, and the like) (e.g., the therapeutic agent is exogenous to the intended site of therapy). In some embodiments, the level of the therapeutic agent is not naturally occurring in the enucleated cell of the parent cell.

In some embodiments, the therapeutic agent is naturally expressed (e.g., in the absence of engineering) in the cell from which the enucleated cell was derived or obtained (e.g., the therapeutic agent is endogenous to the enucleated cell). In some embodiments, the therapeutic agent is naturally expressed in the subject (e.g., the therapeutic agent is endogenous to the subject). In some embodiments, therapeutic agent is naturally expressed in the subject at the intended site of therapy (e.g., a tumor, or a particular tissue, such as the brain, the intestine, the lungs, the heart, the liver, the spleen, the pancreas, muscles, eyes, and the like) (e.g., the therapeutic agent is endogenous to the intended site of therapy).

In some embodiments, the therapeutic agent is derived from a synthetic cell and loaded into the enucleated cell. For example, the therapeutic agent may be endocytosed into the cell. Alternatively, the therapeutic agent may be synthesized by the cell and subsequently delivered to a target cell.

In some embodiments, the therapeutic agent comprises a corrected, a truncated, or a non-mutated version and/or copy of the DNA molecule, the RNA molecule, the protein, the peptide, the small molecule active agent, and/or the gene-editing factor as compared to the cell from which the enucleated cell was derived or obtained. For example, the therapeutic agent can correct a mutated p53 or EGFR in the target cell as part of the treatment for lung cancer.

In some embodiments, therapeutic agent comprises at least 2 (e.g., at least 2, 3, 4, 5, or more) different therapeutic DNA molecules, therapeutic RNA molecules, therapeutic proteins, therapeutic peptides, small molecule active agents, or therapeutic gene-editing factors, in any combination. For example, in some embodiments, a therapeutic agent comprises a therapeutic DNA molecule and a small molecule active agent. For example, in some embodiments, the therapeutic agent comprises two different small molecule active agents. For example, in some embodiments, the therapeutic agent comprises a chemokine receptor (e.g., for targeting) and a small molecule active agent.

In some embodiments, the therapeutic agent comprises an RNA molecule comprising messenger RNA (mRNA), short hairpin RNA (shRNA), small interfering RNA (siRNA), microRNA, long non-coding RNA (lncRNA) or an RNA virus. In some embodiments, the therapeutic agent comprises a DNA molecule that is single-stranded DNA, double-stranded DNA, an oligonucleotide, a plasmid, a bacterial DNA molecule or a DNA virus. In some embodiments, the therapeutic agent comprises a protein, or a portion thereof. In some embodiments, the protein is a cytokine, a growth factor, a hormone, an antibody or an antigen-binding fragment thereof, a small-peptide based drug, or an enzyme. In some embodiments, the enucleated cell transiently expresses the therapeutic agent. In some embodiments, the expression of the therapeutic agent is inducible. In some embodiments, the expression of the therapeutic agent permanent.

In some embodiments, the therapeutic agent comprises an exogenous agent. In some embodiments, the exogenous agent is an exogenous polypeptide. In some embodiments, the exogenous polypeptide is encoded by an exogenous polynucleotide delivered into the parent cell or the enucleated cell. In some embodiments, the exogenous polypeptide is synthesized or released by at least one intracellular organelle of the enucleated cell. In some embodiments, the exogenous polypeptide is released by the enucleated cell. In some embodiments, the exogenous polypeptide is expressed on the cell surface or the enucleated cell. In some embodiments, the enucleated cell delivers the exogenous polypeptide to a target cell. In some embodiments, the target cell is a cancer cell expressing the cancer biomarker of any cancer described herein. In some embodiments, the target cell is an endothelial cell expressing an endothelial biomarker described herein. In some embodiments, the endothelial cell is a blood vessel cell. In some embodiments, the endothelial cell is a lymphatic vessel cell.

In some embodiments, the exogenous polypeptide comprises a cytokine of any one of the cytokine described herein. In some embodiments, the exogenous polypeptide comprises a soluble cytokine. For example, the exogenous polypeptide can comprise an extracellular domain or fragment of the cytokine. In some embodiments, the exogenous polypeptide comprises a solubility as determined by turbidimetric solubility assay or thermodynamic solubility assay by dissolving the exogenous polypeptide in solvent such as organic solvent, including dimethyl sulfoxide (DMSO), dimethylformamide (DMF), acetonitrile, etc., or inorganic solvent, including water or phosphate-buffered saline (PBS). In some embodiments, the exogenous polypeptide comprises a solubility that is at least 0.0001 mg/ml, 0.0005 mg/ml, 0.001 mg/ml, 0.005 mg/ml, 0.01 mg/ml, 0.05 mg/ml, 0.1 mg/ml, 0.5 mg/ml, 1.0 mg/ml, 5.0 mg/ml, 10 mg/ml, 50 mg/ml, 100 mg/ml, 500 mg/ml 1,000 mg/ml 5,000 mg/ml, 10,000 mg/ml, 50,000 mg/ml, or 100,000 mg/ml.

In some embodiments, the exogenous polypeptide comprises a tumor necrosis factor (TNF) superfamily member or a catalytically active fragment thereof. Non-limiting examples of the TNF superfamily member include Lymphotoxin alpha (TNFβ), Tumor necrosis factor (TNFα), Lymphotoxin beta (TNFγ), OX40 ligand (CD252, Gp34, or CD134L), CD40 ligand (CD154, TRAP, Gp39, or T-BAM), Fas ligand (CD178, APTL, or CD95L), CD27 ligand (CD70), CD30 ligand (CD153), CD137 ligand (4-1 BBL), TNF-related apoptosis-inducing ligand (CD253 or APO-2L), Receptor activator of nuclear factor kappa-B ligand (CD254, OPGL, TRANCE, or ODF), TNF-related weak inducer of apoptosis (APO-3L or DR3L), a proliferation-inducing ligand (CD256, TALL-2, or TRDL1), B-cell activating factor (CD257, BLyS, TALL-1, or TNFSF20), LIGHT (CD258 or HVEML), Vascular endothelial growth inhibitor (TL1 or TL-1A), TNF superfamily member 18 (GITRL, AITRL, or TL-6), or Ectodysplasin A (ED1-A1 or ED1-A2).

In some embodiments, the therapeutic agent comprises any one of the immune checkpoint proteins described herein or an immune checkpoint inhibitor for inhibiting any one of the immune checkpoint protein described herein. Non-limiting examples of the immune checkpoint protein include PD-1, PD-L1, CTLA-4, VISTA, B7-H3 (also called CD276), A2AR, CD27, LAG3, TIM-3, T cell immunoreceptor with Ig and ITIM domains (TIGIT), CD73, NKG2A, PVRIG, PVRL2, CEACAMI, CEACAM5, CEACAM6, FAK, CCR-2, CCL-2, LIF, CD47, SIRPα, M-CSF, CSF-1R, IL-3, IL-1RAP, IL-8, SEMA4D, Angiopoietin-2, CLEVER-1, Axl, phosphatidylserine or a fragment thereof.

In some embodiments, the enucleated cells comprise an additional therapeutic agent, such as those disclosed herein. In some embodiments, the composition comprising the enucleated cells is formulated for administration to a subject disclosed herein with an additional therapeutic agent. In some embodiments, the additional therapeutic agent is administered to the subject sequentially, simultaneously, substantially sequentially, or substantially simultaneously.

### Biomolecular Suicide Switch

Described herein are enucleated cells comprising one or more biomolecule that induced cell death, such as a biomolecular suicide switch disclosed herein. In some embodiments, the biomolecule is encoded by a heterologous polynucleotide. In some embodiments, the heterologous polynucleotide comprises a promoter configured to activate transcription of the heterologous polynucleotide under conditions sufficient to express the biomolecular suicide switch. In some embodiments, expression of the biomolecular suicide switch is sufficient to cause cell death. In some embodiments, the heterologous polynucleotide is integrated into chromosome of the nucleated cell. In some embodiments, the heterologous polynucleotide comprises a vector. In some embodiments, the heterologous polynucleotide is not integrated into the chromosome of the cell. In such instances, the heterologous polynucleotide can be induced for expression of the heterologous gene produce in absence of a nucleus. For example, the heterologous polynucleotide not integrated into the chromosome of the cell can be induced in nucleated or enucleated cell.

In some embodiments, the promoter driving expression of the biomolecular suicide switch can be compatible with mammalian gene expression, provide rapid, strong gene expression, in the presence of its induction stimulus. In some embodiments, the heterologous gene product is a suicide gene or any gene product that induces cell death. Non-limiting examples of suicide genes may include, but are not limited to, caspases, DNA crosslinkers, death inducing synthetic NOTCH receptors, toxins, and inductions gents for inducing apoptosis, autophagy, entosis, necrosis, necroptosis, ferroptosis, or a combination thereof.

In some embodiments, the inducible promoter is a hypothermic promoter. In some embodiments, the inducible promoter is induced by contacting the nucleated cells with a temperature that is below about 40° C. In some embodiments, the inducible promoter is induced by contacting the nucleated cells with a temperature that is below about 39° C. In some embodiments, the inducible promoter is induced by contacting the nucleated cells with a temperature that is below about 38° C. In some embodiments, the inducible promoter is induced by contacting the nucleated cells with a temperature that is below about 37° C. In some embodiments, the inducible promoter is induced by contacting the nucleated cells with a temperature that is below about 36° C. In some embodiments, the inducible promoter is induced by contacting the nucleated cells with a temperature that is below about 35° C. In some embodiments, examples of the inducible promoter include, but not limited to, dsrA or CIRP.

In some embodiments, the inducible promoter is a hyperthermic promoter. In some embodiments, the inducible promoter is induced by contacting the nucleated cells with a temperature that is above 35° C. In some embodiments, the inducible promoter is induced by contacting the nucleated cells with a temperature that is above 36° C. In some embodiments, the inducible promoter is induced by contacting the nucleated cells with a temperature that is above 37° C. In some embodiments, the inducible promoter is induced by contacting the nucleated cells with a temperature that is above 38° C. In some embodiments, the inducible promoter is induced by contacting the nucleated cells with a temperature that is above 39° C. In some embodiments, the inducible promoter is induced by contacting the nucleated cells with a temperature that is above 40° C. In some embodiments, examples of the inducible promoter include, but not limited to, HSP70, HSP90, GADD153, MDR1, or HSE-CMV.

In some embodiments, the inducible promoter is induced by contacting the nucleated cells with a molecule. In some embodiments, examples of the molecule include, but not limited to, rtTA, TRE, TetR, Cumate, Rapamycin, Abscisic acid, IPTG, or Methallothionein.

In some embodiments, the inducible promoter is induced by contacting the nucleated cells with light. In some embodiments, examples of the inducible promoter include, but not limited to, CIB1-CRY2 or GAL4-VVD.

In some embodiments, the inducible promoter is induced by contacting with the nucleated cells with a hormone. In some embodiments, examples of the inducible promoter include, but not limited to, Estradiol-Gal4.

In some embodiments, the promoter comprises a constitutively active promoter. The promoter will be continually active, but suicide will be induced under certain circumstances. In some embodiments, the constitutively active promoter is configured to activate transcription of the heterologous polynucleotide under conditions sufficient to express the heterologous gene product. In some embodiments, examples of the heterologous gene product include, but not limited to, herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase. In some embodiments, examples of the heterologous gene product include, but not limited to, FKBP or a caspase. In some embodiments, an example of heterologous gene product includes, but not limited to, an antigen. In some embodiments, the heterologous polynucleotide is integrated into chromosome of the nucleated cells. In some embodiments, an example of the heterologous polynucleotide includes, but not limited to, a vector.

### Pharmaceutical Compositions

Disclosed herein, in some aspects, are pharmaceutical compositions comprising the compositions disclosed herein and a pharmaceutically acceptable: carrier, excipient, diluent, or nebulized inhalant. In some embodiments, the compositions disclosed herein comprise one or more active agents or therapeutic agents.

In some embodiments, the compositions comprise two or more active agents, or two or more therapeutic agents as disclosed herein. In some embodiments, the two or more active agents are contained in a single dosage unit, such as for example, when the enucleated cell comprises two or more therapeutic agents. In embodiments, the two or more active agents are contained in separate dosage units, such as when the enucleated cell is administered separately from an additional therapeutic agent or adjuvant. In some embodiments, the pharmaceutical composition described herein includes at least one additional active agent other than the enucleated cell described herein. In some embodiments, the at least one additional active agent is a chemotherapeutic agent, cytotoxic agent, cytokine, growth-inhibitory agent, anti-hormonal agent, anti-angiogenic agent, cardio protectant, and/or checkpoint inhibitor. Non-limiting checkpoint inhibitor includes IMP321/Eftilagimod alpha (Immutep), Relatlimab BMS-986016, Ipilimumab (Yervoy), Pembrolizumab (Keytruda), Nivolumab (Opdivo), Cemiplimab (Libtayo), Atezolizumab (Tecentriq), Avelumab (Bavencio), Durvalumab (Imfinzi), Ipilimumab (Yervoy), LAG525, MK-4280, Irinotecan, Oxaliplatin, REGN3767, TSR-033, B1754111, Sym022, FS118 (a bi-specific anti-LAG3/PD-L1 antagonistic mAb), MGD013 (a bi-specific anti-LAG3/PD-1 antagonistic mAb), TSR-022, Niraparib, Bevacizumab, MBG453, Decitabine, Spartalizumab, Sym023, INCAGN2390, LY3321367, Ramucirumab, Abemaciclib, Merestinib, BMS-986258, SHR-1702, Camrelizumab, MK-7684, Etigilimab/OMP-313 M32, Tiragolumab/MTIG7192A/RG-6058, BMS-986207, AB-154, ASP-8374, JNJ-61610588, CA-170d, Enoblituzumab/MGA271, MGD009, I-8H9/omburtamab,Trastuzumab, MGD013 (Anti-PD-1, anti-LAG-3 dual checkpoint inhibitor), BGB-A1217, CM-24 (MK-6018), BMS 986178, MEDI6469, PF-04518600, GSK3174998, MOXR0916, Utomilimab (PF-05082566), Urelumab (BMS-663513) ES101, BMS-986156, TRX-518, AMG 228, JTX-2011, GSK3359609, BMS-986226, MEDI-570, or Varlilumab (CDX-1127). Such compounds or drugs may be present in combination in amounts that are effective for the purpose intended. Additional non-limiting examples of the additional therapeutic agent include CPI-006 (for inhibiting CD73 and allowing T cell and APC activation); Monalizumab (for inhibiting NKG2A); COM701 (for inhibiting PVRIG/PVRL2 and activating T cell); CM24 (for inhibiting CEACAMI and allowing T and NK cells activation); NEO-201 (for inhibiting CEACAM5 and CEACAM6 which allows T cell activation while interfering with tumor cell growth); Defactinib (for inhibiting FAK and interfering with tumor growth); PF-04136309 (for inhibiting CCR-2 and CCL-2 and allowing T cell recruitment and activation); MSC-1 (for inhibiting LIF and allowing T cell and APC activation while interfering with cancer growth); Hu5F9-G4 (5F9), ALX148, TTI-662, and RRx-001 (for inhibiting CD47 or SIRPa and allowing T cell and APC activation); Lacnotuzumab (MCS-110), LY3022855, SNDX-6352, Emactuzumab (RG7155), and Pexidartinib (PLX3397) (for inhibiting M-CSF or CSF-1R and allowing APC activation); CAN04 and Canakinumab (ACZ885) (for inhibiting IL-3 or IL-1RAP and allowing T cell and APC activation); BMS-986253 (for inhibiting IL-8 and decreasing immunosuppressive tumor microenvironment while interfering with tumor growth); Pepinemab (VX15/2503) (for inhibiting SEMA4D and decreasing immunosuppressive tumor microenvironment while interfering with tumor growth); Trebananib (for inhibiting Angiopoietin-2 and allowing APC activation while interfering with cancer growth); FP-1305 (for inhibiting CLEVER-1 and allowing APC activation); Enapotamab vedotin (EnaV) (for inhibiting Axl and allowing APC activation while interfering with cancer growth); or Bavituximab (for inhibiting phosphatidylserine and allowing T cell and APC activation while interfering with cancer growth).

The compositions may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, compositions described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, I about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Formulations for Administration

The compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

For intravenous injections or drips or infusions, a composition described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Compositions for injection may be presented in unit dosage form, e.g., in ampoules or in multi dose containers, with an added preservative. The composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch. Formulations that include a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, the compositions of the exogenous therapeutic agents are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

Compositions for oral administration are in dosages suitable for such administration. In one aspect, solid oral dosage forms are prepared by mixing a composition with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the composition is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating may provide a delayed release of a therapeutic agent from the formulation. In other embodiments, the film coating aids in patient compliance. Film coatings may range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the compositions described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

The compositions (e.g., pharmaceutical compositions) described herein may be formulated for administration to a subject by administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended-release formulations, pulsatile release formulations, multiparticulate formulations, and mixed immediate and controlled release formulations.

Buccal formulations are administered using a variety of formulations known in the art. In addition, the buccal dosage forms described herein may further include a bioerodible (hydrolysable) polymeric carrier that also serves to adhere the dosage form to the buccal mucosa. For buccal or sublingual administration, the compositions may take the form of tablets, lozenges, or gels formulated in a suitable manner.

For intravenous injections, a composition is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a composition described herein is in a form suitable for parenteral injection as a sterile suspensions, solutions or emulsions in oily or aqueous vehicles, and contain formulatory agents such as suspending, stabilizing and/or dispersing agents. The compositions for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Suitable formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, the compositions are provided that include particles of a therapeutic agent and at least one dispersing agent or suspending agent for oral administration to a subject. The formulations may be a powder and/or granule for suspension, and upon admixture with water, a substantially uniform suspension is obtained.

Furthermore, the compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other the compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

An aerosol formulation for nasal administration is generally an aqueous solution designed to be administered to the nasal passages in drops or sprays. Nasal solutions may be similar to nasal secretions in that they are generally isotonic and slightly buffered to maintain a pH of about 5.5 to about 6.5, although pH values outside of this range may additionally be used. Antimicrobial agents or preservatives may also be included in the formulation.

An aerosol formulation for inhalations and inhalants may be designed so that the agent or combination of agents is carried into the respiratory tree of the subject when administered by the nasal or oral respiratory route. Inhalation solutions may be administered, for example, by a nebulizer. Inhalations or insufflations, comprising finely powdered or liquid drugs, may be delivered to the respiratory system as a pharmaceutical aerosol of a solution or suspension of the agent or combination of agents in a propellant, e.g., to aid in disbursement. Propellants may be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Aerosol formulations may also include other components, for example, ethanol, isopropanol, propylene glycol, as well as surfactants or other components such as oils and detergents. These components may serve to stabilize the formulation and/or lubricate valve components.

The aerosol formulation may be packaged under pressure and may be formulated as an aerosol using solutions, suspensions, emulsions, powders and semisolid preparations. For example, a solution aerosol formulation comprises a solution of an agent such as a transporter, carrier, or ion channel inhibitor in (substantially) pure propellant or as a mixture of propellant and solvent. The solvent may be used to dissolve the agent and/or retard the evaporation of the propellant. Solvents may include, for example, water, ethanol and glycols. Any combination of suitable solvents may be use, optionally combined with preservatives, antioxidants, and/or other aerosol components.

An aerosol formulation may be a dispersion or suspension. A suspension aerosol formulation comprises a suspension of an agent or combination of agents, e.g., a transporter, carrier, or ion channel inhibitor, and a dispersing agent. Dispersing agents may include, for example, sorbitan trioleate, oleyl alcohol, oleic acid, lecithin and corn oil. A suspension aerosol formulation may also include lubricants, preservatives, antioxidant, and/or other aerosol components.

An aerosol formulation may similarly be formulated as an emulsion. An emulsion aerosol formulation may include, for example, an alcohol such as ethanol, a surfactant, water and a propellant, as well as an agent or combination of agents, e.g., a transporter, carrier, or ion channel. The surfactant used may be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

### Methods

Disclosed herein, in some embodiments, are methods of producing or using the compositions disclosed herein. In some embodiments, methods comprise high throughput techniques for enucleated cells to produce compositions comprising enucleated cells for biomedical applications with minimal residual nucleated parent cells. In some embodiments, methods comprising inducing expression of a suicide gene under conditions suitable to kill the residual nucleated parent cells in the composition. The methods disclosed herein also provide methods of using the enucleated cells as fusion partners (e.g., fusion to another cell in vivo or ex vivo), or a therapeutic agent delivery vehicle, or a combination thereof.

Disclosed herein, in some aspects, are methods of producing an enucleated cell described herein comprising enucleating a nucleated parent cell. In some embodiments, the parent cell may be treated with an exogenous molecule to soften cytoskeleton of the parent cell. For example, the parent cell can be treated with cytochalasin to soften the cortical actin cytoskeleton. In some embodiments, the nucleus is physically extracted from the cell body by centrifugation to generate an enucleated cell. In some embodiments, the centrifugation comprises use of density gradients, where the enucleated cells are isolated at least because the enucleate cells and intact nucleated cells sediment to different layers in the density gradient. In some embodiments, the centrifugation comprises continuous-flow centrifugation. Example 8 illustrates an exemplary continuous flow centrifugation experiment for obtaining enucleated cells from nucleated cells. In some embodiments, the continuous-flow centrifugation is fixed angle centrifugation. In some embodiments, the use of continuous-flow centrifugation increases the volume that can be centrifuged. For example, the use of continuous-flow centrifugation increases a volume that can be centrifuged compared to swinging-bucket centrifugation (for generating a comparable density gradient). In some embodiments, the centrifugation comprises zonal centrifugation, where the enucleated cell is separated from the nucleated cell based on a difference in size, difference in mass, or a combination thereof. Example 7 illustrates generating enucleated cells by zonal centrifugation. In some embodiments, the method comprises inducing cell death of the nucleated cells after the centrifugation or enucleation. For example, the nucleated cells can be engineered to possess a heterologous polynucleotide encoding a heterologous gene product described herein, where expression of the heterologous gene product induces cell death of at least one nucleated cell.

In some embodiments, methods disclosed herein result in a composition comprising tens of millions of enucleated cells (“enucleated cell fraction”). In some embodiments, the composition also includes residual nucleated cells (“nucleated cell fraction”). In some embodiments, the composition is further processed to purify the enucleated cell fraction from the nucleated cell fraction. In some embodiments, the enucleated cell fraction is formulated in a pharmaceutical composition comprising a pharmaceutically acceptable: carrier, excipient, or diluent.

In some embodiments, methods of producing the enucleated cell do not consist or comprise of differentiation of the parent cell. For example, the enucleated cell is not obtained by differentiating a nucleated erythroid progenitor cell into a differentiated and enucleated red blood cell. In some embodiments, the enucleated cell is not a terminally differentiated cell. In some embodiments, the enucleated cell is not a platelet. In some embodiments, the enucleated cell is not obtained from a platelet lineage cell. In some embodiments, the enucleated cell is not a red blood cell. In some embodiments, the enucleated cell is not obtained from a red blood cell lineage cell.

In some embodiments, the parent cell containing a nucleus is engineered to express at least one of therapeutic agent, transmembrane moiety, immune-evading moiety, or targeting moiety described herein; and subsequently, the nucleus of the parent cell is removed. In some embodiments, the parent cell containing the nucleus is enucleated, and the enucleated cell is engineered to express therapeutic agent, transmembrane moiety, immune-evading moiety, or targeting moiety described herein. In some embodiments, the parent cell is engineered to express one or more of the biomolecules above (e.g., immune-evading moiety and/or targeting moiety), and the resulting enucleated cell (e.g., already expressing the immune-evading moiety and/or targeting moiety) is further engineered to express a second of the biomolecules above (e.g., a therapeutic agent). In this manner, the enucleated cells of the present disclosure can be extensively engineered prior to enucleation, stored for long periods of time as needed (through for e.g., lyophilization, cryohibemation, cryopreservation), and quickly engineered to express a therapeutic agent closer to the time of need.

In some embodiments, the composition has the volume comprising more than or equal to about 10 milliliters (mL) to about 10,000 mL. In some embodiments, the composition has the volume comprising more than or equal to about 10 mL to about 100 mL, about 10 mL to about 1,000 mL, about 10 mL to about 2,000 mL, about 10 mL to about 3,000 mL, about 10 mL to about 4,000 mL, about 10 mL to about 5,000 mL, about 10 mL to about 6,000 mL, about 10 mL to about 7,000 mL, about 10 mL to about 8,000 mL, about 10 mL to about 9,000 mL, about 10 mL to about 10,000 mL, about 100 mL to about 1,000 mL, about 100 mL to about 2,000 mL, about 100 mL to about 3,000 mL, about 100 mL to about 4,000 mL, about 100 mL to about 5,000 mL, about 100 mL to about 6,000 mL, about 100 mL to about 7,000 mL, about 100 mL to about 8,000 mL, about 100 mL to about 9,000 mL, about 100 mL to about 10,000 mL, about 1,000 mL to about 2,000 mL, about 1,000 mL to about 3,000 mL, about 1,000 mL to about 4,000 mL, about 1,000 mL to about 5,000 mL, about 1,000 mL to about 6,000 mL, about 1,000 mL to about 7,000 mL, about 1,000 mL to about 8,000 mL, about 1,000 mL to about 9,000 mL, about 1,000 mL to about 10,000 mL, about 2,000 mL to about 3,000 mL, about 2,000 mL to about 4,000 mL, about 2,000 mL to about 5,000 mL, about 2,000 mL to about 6,000 mL, about 2,000 mL to about 7,000 mL, about 2,000 mL to about 8,000 mL, about 2,000 mL to about 9,000 mL, about 2,000 mL to about 10,000 mL, about 3,000 mL to about 4,000 mL, about 3,000 mL to about 5,000 mL, about 3,000 mL to about 6,000 mL, about 3,000 mL to about 7,000 mL, about 3,000 mL to about 8,000 mL, about 3,000 mL to about 9,000 mL, about 3,000 mL to about 10,000 mL, about 4,000 mL to about 5,000 mL, about 4,000 mL to about 6,000 mL, about 4,000 mL to about 7,000 mL, about 4,000 mL to about 8,000 mL, about 4,000 mL to about 9,000 mL, about 4,000 mL to about 10,000 mL, about 5,000 mL to about 6,000 mL, about 5,000 mL to about 7,000 mL, about 5,000 mL to about 8,000 mL, about 5,000 mL to about 9,000 mL, about 5,000 mL to about 10,000 mL, about 6,000 mL to about 7,000 mL, about 6,000 mL to about 8,000 mL, about 6,000 mL to about 9,000 mL, about 6,000 mL to about 10,000 mL, about 7,000 mL to about 8,000 mL, about 7,000 mL to about 9,000 mL, about 7,000 mL to about 10,000 mL, about 8,000 mL to about 9,000 mL, about 8,000 mL to about 10,000 mL, or about 9,000 mL to about 10,000 mL. In some embodiments, the composition has the volume comprising more than or equal to about 10 mL, about 100 mL, about 1,000 mL, about 2,000 mL, about 3,000 mL, about 4,000 mL, about 5,000 mL, about 6,000 mL, about 7,000 mL, about 8,000 mL, about 9,000 mL, or about 10,000 mL. In some embodiments, the composition has the volume comprising more than or equal to at least about 10 mL, about 100 mL, about 1,000 mL, about 2,000 mL, about 3,000 mL, about 4,000 mL, about 5,000 mL, about 6,000 mL, about 7,000 mL, about 8,000 mL, or about 9,000 mL. In some embodiments, the composition has the volume comprising more than or equal to at most about 100 mL, about 1,000 mL, about 2,000 mL, about 3,000 mL, about 4,000 mL, about 5,000 mL, about 6,000 mL, about 7,000 mL, about 8,000 mL, about 9,000 mL, or about 10,000 mL.

In some embodiments, the composition has a volume comprising between about 10 mL to about 10,000 mL. In some embodiments, the composition has a volume comprising between about 10 mL to about 100 mL, about 10 mL to about 1,000 mL, about 10 mL to about 2,000 mL, about 10 mL to about 3,000 mL, about 10 mL to about 4,000 mL, about 10 mL to about 5,000 mL, about 10 mL to about 6,000 mL, about 10 mL to about 7,000 mL, about 10 mL to about 8,000 mL, about 10 mL to about 9,000 mL, about 10 mL to about 10,000 mL, about 100 mL to about 1,000 mL, about 100 mL to about 2,000 mL, about 100 mL to about 3,000 mL, about 100 mL to about 4,000 mL, about 100 mL to about 5,000 mL, about 100 mL to about 6,000 mL, about 100 mL to about 7,000 mL, about 100 mL to about 8,000 mL, about 100 mL to about 9,000 mL, about 100 mL to about 10,000 mL, about 1,000 mL to about 2,000 mL, about 1,000 mL to about 3,000 mL, about 1,000 mL to about 4,000 mL, about 1,000 mL to about 5,000 mL, about 1,000 mL to about 6,000 mL, about 1,000 mL to about 7,000 mL, about 1,000 mL to about 8,000 mL, about 1,000 mL to about 9,000 mL, about 1,000 mL to about 10,000 mL, about 2,000 mL to about 3,000 mL, about 2,000 mL to about 4,000 mL, about 2,000 mL to about 5,000 mL, about 2,000 mL to about 6,000 mL, about 2,000 mL to about 7,000 mL, about 2,000 mL to about 8,000 mL, about 2,000 mL to about 9,000 mL, about 2,000 mL to about 10,000 mL, about 3,000 mL to about 4,000 mL, about 3,000 mL to about 5,000 mL, about 3,000 mL to about 6,000 mL, about 3,000 mL to about 7,000 mL, about 3,000 mL to about 8,000 mL, about 3,000 mL to about 9,000 mL, about 3,000 mL to about 10,000 mL, about 4,000 mL to about 5,000 mL, about 4,000 mL to about 6,000 mL, about 4,000 mL to about 7,000 mL, about 4,000 mL to about 8,000 mL, about 4,000 mL to about 9,000 mL, about 4,000 mL to about 10,000 mL, about 5,000 mL to about 6,000 mL, about 5,000 mL to about 7,000 mL, about 5,000 mL to about 8,000 mL, about 5,000 mL to about 9,000 mL, about 5,000 mL to about 10,000 mL, about 6,000 mL to about 7,000 mL, about 6,000 mL to about 8,000 mL, about 6,000 mL to about 9,000 mL, about 6,000 mL to about 10,000 mL, about 7,000 mL to about 8,000 mL, about 7,000 mL to about 9,000 mL, about 7,000 mL to about 10,000 mL, about 8,000 mL to about 9,000 mL, about 8,000 mL to about 10,000 mL, or about 9,000 mL to about 10,000 mL. In some embodiments, the composition has a volume comprising between about 10 mL, about 100 mL, about 1,000 mL, about 2,000 mL, about 3,000 mL, about 4,000 mL, about 5,000 mL, about 6,000 mL, about 7,000 mL, about 8,000 mL, about 9,000 mL, or about 10,000 mL. In some embodiments, the composition has a volume comprising between at least about 10 mL, about 100 mL, about 1,000 mL, about 2,000 mL, about 3,000 mL, about 4,000 mL, about 5,000 mL, about 6,000 mL, about 7,000 mL, about 8,000 mL, or about 9,000 mL. In some embodiments, the composition has a volume comprising between at most about 100 mL, about 1,000 mL, about 2,000 mL, about 3,000 mL, about 4,000 mL, about 5,000 mL, about 6,000 mL, about 7,000 mL, about 8,000 mL, about 9,000 mL, or about 10,000 mL.

In some embodiments, described herein are methods for cell processing by enucleating a portion of the nucleated cells (parent cells) to produce an enucleated cell fraction using continuous flow centrifugation, where the continuous flow centrifugation is fixed angle centrifugation. In some embodiments, the continuous flow centrifugation is swinging bucket centrifugation.

In some embodiments, the resulting composition comprises an enucleated cell fraction, which may be 100% of the composition. In other embodiments, there may be a nucleated cell fraction of the composition comprised of nucleated parent cells that were not enucleated. In some embodiments, the enucleated cell fraction is greater than or equal and about 30% composition. In some embodiments, the enucleated cell fraction is greater than or equal to about 35% of the composition. In some embodiments, the enucleated cell fraction is greater than or equal to about 40% of the composition. In some embodiments, the enucleated cell fraction is greater than or equal to about 45% of the composition. In some embodiments, the enucleated cell fraction is greater than or equal to about 50% of the composition. In some embodiments, the enucleated cell fraction is greater than or equal to about 55% of the composition. In some embodiments, the enucleated cell fraction is greater than or equal to about 60% of the composition. In some embodiments, the enucleated cell fraction is greater than or equal to about 65% of the composition. In some embodiments, the enucleated cell fraction is greater than or equal to about 70% of the composition. In some embodiments, the enucleated cell fraction is greater than or equal to about 75% of the composition. In some embodiments, the enucleated cell fraction is greater than or equal to about 80% of the composition. In some embodiments, the enucleated cell fraction is greater than or equal to about 85% of the composition. In some embodiments, the enucleated cell fraction is greater than or equal to about 90% of the composition. In some embodiments, the enucleated cell fraction is greater than or equal to about 95% of the composition. In some embodiments, the enucleated cell fraction is greater than or equal to about 96% of the composition. In some embodiments, the enucleated cell fraction is greater than or equal to about 97% of the composition. In some embodiments, the enucleated cell fraction is greater than or equal to about 98% of the composition. In some embodiments, the enucleated cell fraction is greater than or equal to about 99% of the composition.

In some embodiments, cell separation, cell isolation, or cell sorting, is a process to isolate one or more specific cell populations from a heterogeneous mixture of cells. In some embodiments, the methods of enucleating cells disclosed herein is performed on an isolated population of homogenous cells. In some embodiments, the methods of enucleated cells disclosed herein is performed on a heterogeneous mixture of cells. In some embodiments, methods disclosed herein comprise isolating a population of homogenous cells from a mixture of heterogenous cells using a suitable cell separation technique including, but not limited to, immunomagnetic cell separation, fluorescence-activated cell sorting, density gradient centrifugation, immunedensity cell isolation, microfluidic cell sorting, buoyancy-activated cell sorting, aptamer-based cell isolation, complement depletion, or any combination thereof.

In centrifugation, more dense particles can move to the outer edges of the mixture while less dense objects groups together further in as the sample is spun. A biological sample can be centrifuged until the cell types are isolated into layers. During centrifugation, each cell type can sediment to its isopycnic point, which is the place in the medium gradient, where the density of the cells and medium are equal. Examples of density gradient media include Lymphoprep™ Lympholyte®, Ficoll-Paque®, Percoll®, OptiPrep™, Cell Separation with Accuspin™ Aystem-Histopaque® Media, Histopaque® Media, Histopaque® Iodinated Gradient Media, inorganic salts, nonionic iodinated density gradient media, polyhydric alcohols, polysaccharides, etc. For instance, Lymphoprep™, Lympholyte®, and Ficoll-Paque® consists of saccharides and sodium diatrizoate and may be used to isolate mononuclear cells from peripheral blood, cord blood, and bone marrow. Percoll® consists of colloidal silica particles coated with polyvinylpyrrolidone and is widely used to separate cells, organelles, viruses, and other subcellular particles. OptiPrep™ is a medium consisting of iodixanol in water and used to isolate viruses, organelles, macromolecules, and cells.

In some embodiments, disclosed herein are methods for cell processing by enucleating a portion of the nucleated cells to produce an enucleated cell fraction using continuous flow centrifugation. In some embodiments, disclosed herein are methods for cell processing by enucleating a portion of the nucleated cells to produce an enucleated cell fraction using zonal centrifugation. In some embodiments, the continuous flow centrifugation is fixed angle centrifugation. In some embodiments, the continuous flow centrifugation is swinging bucket centrifugation. In some embodiments, the continuous flow centrifugation generates a density gradient. In some embodiments, the density gradient separates the enucleated cell fraction from the nucleated cells in the composition. In some embodiments, the density gradient comprises a polysaccharide density gradient. In some embodiments, the polysaccharide density gradient comprises a Ficoll density gradient. In some embodiments, methods further comprise producing the Ficoll gradient by polymerizing sucrose molecules with epichlorohydrin to give a polysaccharide that is osmotically inert.

In some embodiments, the gradient comprises between 2 ranges to 20 ranges of the density gradient. In some embodiments, the gradient comprises between 2 ranges to 3 ranges, 2 ranges to 4 ranges, 2 ranges to 5 ranges, 2 ranges to 6 ranges, 2 ranges to 8 ranges, 2 ranges to 10 ranges, 2 ranges to 12 ranges, 2 ranges to 14 ranges, 2 ranges to 16 ranges, 2 ranges to 18 ranges, 2 ranges to 20 ranges, 3 ranges to 4 ranges, 3 ranges to 5 ranges, 3 ranges to 6 ranges, 3 ranges to 8 ranges, 3 ranges to 10 ranges, 3 ranges to 12 ranges, 3 ranges to 14 ranges, 3 ranges to 16 ranges, 3 ranges to 18 ranges, 3 ranges to 20 ranges, 4 ranges to 5 ranges, 4 ranges to 6 ranges, 4 ranges to 8 ranges, 4 ranges to 10 ranges, 4 ranges to 12 ranges, 4 ranges to 14 ranges, 4 ranges to 16 ranges, 4 ranges to 18 ranges, 4 ranges to 20 ranges, 5 ranges to 6 ranges, 5 ranges to 8 ranges, 5 ranges to 10 ranges, 5 ranges to 12 ranges, 5 ranges to 14 ranges, 5 ranges to 16 ranges, 5 ranges to 18 ranges, 5 ranges to 20 ranges, 6 ranges to 8 ranges, 6 ranges to 10 ranges, 6 ranges to 12 ranges, 6 ranges to 14 ranges, 6 ranges to 16 ranges, 6 ranges to 18 ranges, 6 ranges to 20 ranges, 8 ranges to 10 ranges, 8 ranges to 12 ranges, 8 ranges to 14 ranges, 8 ranges to 16 ranges, 8 ranges to 18 ranges, 8 ranges to 20 ranges, 10 ranges to 12 ranges, 10 ranges to 14 ranges, 10 ranges to 16 ranges, 10 ranges to 18 ranges, 10 ranges to 20 ranges, 12 ranges to 14 ranges, 12 ranges to 16 ranges, 12 ranges to 18 ranges, 12 ranges to 20 ranges, 14 ranges to 16 ranges, 14 ranges to 18 ranges, 14 ranges to 20 ranges, 16 ranges to 18 ranges, 16 ranges to 20 ranges, or 18 ranges to 20 ranges of the density gradient. In some embodiments, the gradient comprises between 2 ranges, 3 ranges, 4 ranges, 5 ranges, 6 ranges, 8 ranges, 10 ranges, 12 ranges, 14 ranges, 16 ranges, 18 ranges, or 20 ranges. In some embodiments, the gradient comprises between at least 2 ranges, 3 ranges, 4 ranges, 5 ranges, 6 ranges, 8 ranges, 10 ranges, 12 ranges, 14 ranges, 16 ranges, or 18 ranges of the density gradient. In some embodiments, the gradient comprises between at most 3 ranges, 4 ranges, 5 ranges, 6 ranges, 8 ranges, 10 ranges, 12 ranges, 14 ranges, 16 ranges, 18 ranges, or 20 ranges of the density gradient. In some embodiments, the gradient comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty ranges of the density gradient. In some embodiments, the gradient comprises at least seven ranges of the density gradient. In some embodiments, the gradient comprises at least five ranges of the density gradient. In some embodiments, the gradient comprises at least three ranges of the density gradient. In some embodiments, the gradient comprises seven ranges of the density gradient. In some embodiments, the gradient comprises five ranges of the density gradient. In some embodiments, the gradient comprises three ranges of the density gradient.

In some embodiments, the gradient comprises about 7.5% density gradient media to about 30% density gradient media. 7.5% density gradient media to about 10% density gradient media, about 7.5% density gradient media to about 12.5% density gradient media, about 7.5% density gradient media to about 15% density gradient media, about 7.5% density gradient media to about 16% density gradient media, about 7.5% density gradient media to about 17% density gradient media, about 7.5% density gradient media to about 18% density gradient media, about 7.5% density gradient media to about 19% density gradient media, about 7.5% density gradient media to about 20% density gradient media, about 7.5% density gradient media to about 25% density gradient media, about 7.5% density gradient media to about 27.5% density gradient media, about 7.5% density gradient media to about 30% density gradient media, about 10% density gradient media to about 12.5% density gradient media, about 10% density gradient media to about 15% density gradient media, about 10% density gradient media to about 16% density gradient media, about 10% density gradient media to about 17% density gradient media, about 10% density gradient media to about 18% density gradient media, about 10% density gradient media to about 19% density gradient media, about 10% density gradient media to about 20% density gradient media, about 10% density gradient media to about 25% density gradient media, about 10% density gradient media to about 27.5% density gradient media, about 10% density gradient media to about 30% density gradient media, about 12.5% density gradient media to about 15% density gradient media, about 12.5% density gradient media to about 16% density gradient media, about 12.5% density gradient media to about 17% density gradient media, about 12.5% density gradient media to about 18% density gradient media, about 12.5% density gradient media to about 19% density gradient media, about 12.5% density gradient media to about 20% density gradient media, about 12.5% density gradient media to about 25% density gradient media, about 12.5% density gradient media to about 27.5% density gradient media, about 12.5% density gradient media to about 30% density gradient media, about 15% density gradient media to about 16% density gradient media, about 15% density gradient media to about 17% density gradient media, about 15% density gradient media to about 18% density gradient media, about 15% density gradient media to about 19% density gradient media, about 15% density gradient media to about 20% density gradient media, about 15% density gradient media to about 25% density gradient media, about 15% density gradient media to about 27.5% density gradient media, about 15% density gradient media to about 30% density gradient media, about 16% density gradient media to about 17% density gradient media, about 16% density gradient media to about 18% density gradient media, about 16% density gradient media to about 19% density gradient media, about 16% density gradient media to about 20% density gradient media, about 16% density gradient media to about 25% density gradient media, about 16% density gradient media to about 27.5% density gradient media, about 16% density gradient media to about 30% density gradient media, about 17% density gradient media to about 18% density gradient media, about 17% density gradient media to about 19% density gradient media, about 17% density gradient media to about 20% density gradient media, about 17% density gradient media to about 25% density gradient media, about 17% density gradient media to about 27.5% density gradient media, about 17% density gradient media to about 30% density gradient media, about 18% density gradient media to about 19% density gradient media, about 18% density gradient media to about 20% density gradient media, about 18% density gradient media to about 25% density gradient media, about 18% density gradient media to about 27.5% density gradient media, about 18% density gradient media to about 30% density gradient media, about 19% density gradient media to about 20% density gradient media, about 19% density gradient media to about 25% density gradient media, about 19% density gradient media to about 27.5% density gradient media, about 19% density gradient media to about 30% density gradient media, about 20% density gradient media to about 25% density gradient media, about 20% density gradient media to about 27.5% density gradient media, about 20% density gradient media to about 30% density gradient media, about 25% density gradient media to about 27.5% density gradient media, about 25% density gradient media to about 30% density gradient media, or about 27.5% density gradient media to about 30% density gradient media.

In some embodiments, the gradient is a Ficoll gradient. In some embodiments, the Ficoll gradient comprises between 2 ranges to 20 ranges of the density Ficoll gradient. In some embodiments, the Ficoll gradient comprises between 2 ranges to 3 ranges, 2 ranges to 4 ranges, 2 ranges to 5 ranges, 2 ranges to 6 ranges, 2 ranges to 8 ranges, 2 ranges to 10 ranges, 2 ranges to 12 ranges, 2 ranges to 14 ranges, 2 ranges to 16 ranges, 2 ranges to 18 ranges, 2 ranges to 20 ranges, 3 ranges to 4 ranges, 3 ranges to 5 ranges, 3 ranges to 6 ranges, 3 ranges to 8 ranges, 3 ranges to 10 ranges, 3 ranges to 12 ranges, 3 ranges to 14 ranges, 3 ranges to 16 ranges, 3 ranges to 18 ranges, 3 ranges to 20 ranges, 4 ranges to 5 ranges, 4 ranges to 6 ranges, 4 ranges to 8 ranges, 4 ranges to 10 ranges, 4 ranges to 12 ranges, 4 ranges to 14 ranges, 4 ranges to 16 ranges, 4 ranges to 18 ranges, 4 ranges to 20 ranges, 5 ranges to 6 ranges, 5 ranges to 8 ranges, 5 ranges to 10 ranges, 5 ranges to 12 ranges, 5 ranges to 14 ranges, 5 ranges to 16 ranges, 5 ranges to 18 ranges, 5 ranges to 20 ranges, 6 ranges to 8 ranges, 6 ranges to 10 ranges, 6 ranges to 12 ranges, 6 ranges to 14 ranges, 6 ranges to 16 ranges, 6 ranges to 18 ranges, 6 ranges to 20 ranges, 8 ranges to 10 ranges, 8 ranges to 12 ranges, 8 ranges to 14 ranges, 8 ranges to 16 ranges, 8 ranges to 18 ranges, 8 ranges to 20 ranges, 10 ranges to 12 ranges, 10 ranges to 14 ranges, 10 ranges to 16 ranges, 10 ranges to 18 ranges, 10 ranges to 20 ranges, 12 ranges to 14 ranges, 12 ranges to 16 ranges, 12 ranges to 18 ranges, 12 ranges to 20 ranges, 14 ranges to 16 ranges, 14 ranges to 18 ranges, 14 ranges to 20 ranges, 16 ranges to 18 ranges, 16 ranges to 20 ranges, or 18 ranges to 20 ranges of the density Ficoll gradient. In some embodiments, the Ficoll gradient comprises between 2 ranges, 3 ranges, 4 ranges, 5 ranges, 6 ranges, 8 ranges, 10 ranges, 12 ranges, 14 ranges, 16 ranges, 18 ranges, or 20 ranges. In some embodiments, the Ficoll gradient comprises between at least 2 ranges, 3 ranges, 4 ranges, 5 ranges, 6 ranges, 8 ranges, 10 ranges, 12 ranges, 14 ranges, 16 ranges, or 18 ranges of the density Ficoll gradient. In some embodiments, the Ficoll gradient comprises between at most 3 ranges, 4 ranges, 5 ranges, 6 ranges, 8 ranges, 10 ranges, 12 ranges, 14 ranges, 16 ranges, 18 ranges, or 20 ranges of the density Ficoll gradient. In some embodiments, the Ficoll gradient comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty ranges of the density Ficoll gradient. In some embodiments, the Ficoll gradient comprises at least seven ranges of the density Ficoll gradient. In some embodiments, the Ficoll gradient comprises at least five ranges of the density Ficoll gradient. In some embodiments, the Ficoll gradient comprises at least three ranges of the density Ficoll gradient. In some embodiments, the Ficoll gradient comprises seven ranges of the density Ficoll gradient. In some embodiments, the Ficoll gradient comprises five ranges of the density Ficoll gradient. In some embodiments, the Ficoll gradient comprises three ranges of the density Ficoll gradient.

In some embodiments, the Ficoll density gradient comprises about 7.5% Ficoll to about 10% Ficoll, about 7.5% Ficoll to about 12.5% Ficoll, about 7.5% Ficoll to about 15% Ficoll, about 7.5% Ficoll to about 16% Ficoll, about 7.5% Ficoll to about 17% Ficoll, about 7.5% Ficoll to about 18% Ficoll, about 7.5% Ficoll to about 19% Ficoll, about 7.5% Ficoll to about 20% Ficoll, about 7.5% Ficoll to about 25% Ficoll, about 7.5% Ficoll to about 27.5% Ficoll, about 7.5% Ficoll to about 30% Ficoll, about 10% Ficoll to about 12.5% Ficoll, about 10% Ficoll to about 15% Ficoll, about 10% Ficoll to about 16% Ficoll, about 10% Ficoll to about 17% Ficoll, about 10% Ficoll to about 18% Ficoll, about 10% Ficoll to about 19% Ficoll, about 10% Ficoll to about 20% Ficoll, about 10% Ficoll to about 25% Ficoll, about 10% Ficoll to about 27.5% Ficoll, about 10% Ficoll to about 30% Ficoll, about 12.5% Ficoll to about 15% Ficoll, about 12.5% Ficoll to about 16% Ficoll, about 12.5% Ficoll to about 17% Ficoll, about 12.5% Ficoll to about 18% Ficoll, about 12.5% Ficoll to about 19% Ficoll, about 12.5% Ficoll to about 20% Ficoll, about 12.5% Ficoll to about 25% Ficoll, about 12.5% Ficoll to about 27.5% Ficoll, about 12.5% Ficoll to about 30% Ficoll, about 15% Ficoll to about 16% Ficoll, about 15% Ficoll to about 17% Ficoll, about 15% Ficoll to about 18% Ficoll, about 15% Ficoll to about 19% Ficoll, about 15% Ficoll to about 20% Ficoll, about 15% Ficoll to about 25% Ficoll, about 15% Ficoll to about 27.5% Ficoll, about 15% Ficoll to about 30% Ficoll, about 16% Ficoll to about 17% Ficoll, about 16% Ficoll to about 18% Ficoll, about 16% Ficoll to about 19% Ficoll, about 16% Ficoll to about 20% Ficoll, about 16% Ficoll to about 25% Ficoll, about 16% Ficoll to about 27.5% Ficoll, about 16% Ficoll to about 30% Ficoll, about 17% Ficoll to about 18% Ficoll, about 17% Ficoll to about 19% Ficoll, about 17% Ficoll to about 20% Ficoll, about 17% Ficoll to about 25% Ficoll, about 17% Ficoll to about 27.5% Ficoll, about 17% Ficoll to about 30% Ficoll, about 18% Ficoll to about 19% Ficoll, about 18% Ficoll to about 20% Ficoll, about 18% Ficoll to about 25% Ficoll, about 18% Ficoll to about 27.5% Ficoll, about 18% Ficoll to about 30% Ficoll, about 19% Ficoll to about 20% Ficoll, about 19% Ficoll to about 25% Ficoll, about 19% Ficoll to about 27.5% Ficoll, about 19% Ficoll to about 30% Ficoll, about 20% Ficoll to about 25% Ficoll, about 20% Ficoll to about 27.5% Ficoll, about 20% Ficoll to about 30% Ficoll, about 25% Ficoll to about 27.5% Ficoll, about 25% Ficoll to about 30% Ficoll, or about 27.5% Ficoll to about 30% Ficoll.

In some embodiments, the Ficoll density gradient comprises about 7.5% Ficoll, about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, about 27.5% Ficoll, or about 30% Ficoll. In some embodiments, the Ficoll density gradient comprises at least about 7.5% Ficoll, about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, or about 27.5% Ficoll. In some embodiments, the Ficoll density gradient comprises at most about 10% Ficoll, about 12.5% Ficoll, about 15% Ficoll, about 16% Ficoll, about 17% Ficoll, about 18% Ficoll, about 19% Ficoll, about 20% Ficoll, about 25% Ficoll, about 27.5% Ficoll, or about 30% Ficoll. In some embodiments, the Ficoll density gradient comprises about 25% Ficoll, about 17% Ficoll, about 16% Ficoll, about 15% Ficoll, or about 12.5% Ficoll. In some embodiments, the Ficoll density gradient comprises about 25% Ficoll. In some embodiments, the Ficoll density gradient comprises about 17% Ficoll. In some embodiments, the Ficoll density gradient comprises about 16% Ficoll. In some embodiments, the Ficoll density gradient comprises about 15% Ficoll. In some embodiments, the Ficoll density gradient comprises about 12.5% Ficoll.

In some embodiments, the methods for cell processing disclosed herein include enucleating a portion of the nucleated cells to produce an enucleated cell fraction using continuous flow centrifugation, wherein the portion of the nucleated cells that is enucleated is greater than or equal to about 10% of the nucleated cells. In some embodiment, the portion of the nucleated cells is greater than or equal to about 20% of the nucleated cells. In some embodiment, the portion of the nucleated cells is greater than or equal to about 25% of the nucleated cells. In some embodiment, the portion of the nucleated cells is greater than or equal to about 30% of the nucleated cells. In some embodiment, the portion of the nucleated cells is greater than or equal to about 35% of the nucleated cells. In some embodiment, the portion of the nucleated cells is greater than or equal to about 40% of the nucleated cells. In some embodiment, the portion of the nucleated cells is greater than or equal to about 45% of the nucleated cells. In some embodiment, the portion of the nucleated cells is greater than or equal to about 50% of the nucleated cells. In some embodiment, the portion of the nucleated cells is greater than or equal to about 55% of the nucleated cells. In some embodiment, the portion of the nucleated cells is greater than or equal to about 60% of the nucleated cells. In some embodiment, the portion of the nucleated cells is greater than or equal to about 65% of the nucleated cells. In some embodiment, the portion of the nucleated cells is greater than or equal to about 70% of the nucleated cells. In some embodiment, the portion of the nucleated cells is greater than or equal to about 75% of the nucleated cells. In some embodiment, the portion of the nucleated cells is greater than or equal to about 80% of the nucleated cells. In some embodiment, the portion of the nucleated cells is greater than or equal to about 85% of the nucleated cells. In some embodiment, the portion of the nucleated cells is greater than or equal to about 90% of the nucleated cells. In some embodiment, the portion of the nucleated cells is greater than or equal to about 95% of the nucleated cells.

In some embodiments, the enucleated cell fraction produced by methods disclosed herein comprises more than or equal to about 1×10⁵ enucleated cells, 1×10⁶ enucleated cells, about 1×10⁷ enucleated cells, 3×10⁵ enucleated cells, 5×10⁵ enucleated cells, 7×10⁷ of enucleated cells, 8×10⁷ of enucleated cells, 9×10⁷ of enucleated cells, 10×10⁷ of enucleated cells, 15×10⁷ of enucleated cells, 20×10⁷ of enucleated cells, 50×10⁷ of enucleated cells, 70 x 10⁷ enucleated cells, 90×10⁷ enucleated cells, 100×10⁷ of enucleated cells, 150×10⁷ of enucleated cells, 200×10⁷ of enucleated cells, 250×10⁷ enucleated cells, 300×10⁷ enucleated cells, or 500×10⁷ of enucleated cells.

In some embodiments, the enucleated cell of the enucleated cell fraction has a diameter comprising less than or equal to about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99% of an average diameter of the nucleated cells. In some embodiments, the enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells. In some embodiments, the enucleated cell of the enucleated cell fraction has a diameter comprising less than or equal to about 50% of an average diameter of the nucleated cells. In some embodiments, the enucleated cell of the enucleated cell fraction has a diameter comprising less than or equal to about 60% of an average diameter of the nucleated cells. In some embodiments, the enucleated cell of the enucleated cell fraction has a diameter comprising less than or equal to about 70% of an average diameter of the nucleated cells. In some embodiments, the enucleated cell of the enucleated cell fraction has a diameter comprising less than or equal to about 80% of an average diameter of the nucleated cells. In some embodiments, the enucleated cell of the enucleated cell fraction has a diameter comprising less than or equal to about 90% of an average diameter of the nucleated cells.

In some embodiments, the enucleated cell of the enucleated cell fraction has a diameter comprising more than or equal to about 5 μm, about 10 μm, about 20 μm, about 30 μm, about 40 μm, about 50 μm, about 60 μm, about 70 μm, about 80 μm, or about 90 μm. In some embodiments, the enucleated cell of the enucleated cell fraction has a diameter ranging from about 1 μm to about 10 μm. In some embodiments, the enucleated cell of the enucleated cell fraction has a diameter ranging from about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 9 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 9 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 9 μm, about 3 μm to about 10 μm, about 4 μm to about 5 μm, about 4 μm to about 6 μm, about 4 μm to about 7 μm, about 4 μm to about 8 μm, about 4 μm to about 9 μm, about 4 μm to about 10 μm, about 5 μm to about 6 μm, about 5 μm to about 7 μm, about 5 μm to about 8 μm, about 5 μm to about 9 μm, about 5 μm to about 10 μm, about 6 μm to about 7 μm, about 6 μm to about 8 μm, about 6 μm to about 9 μm, about 6 μm to about 10 μm, about 7 μm to about 8 μm, about 7 μm to about 9 μm, about 7 μm to about 10 μm, about 8 μm to about 9 μm, about 8 μm to about 10 μm, or about 9 μm to about 10 μm. In some embodiments, the enucleated cell of the enucleated cell fraction has a diameter ranging from about 1 μm, about 2 μm, about 3 μm, about 4 μm, about 5 μm, about 6 μm, about 7 μm, about 8 μm, about 9 μm, or about 10 μm. In some embodiments, the enucleated cell of the enucleated cell fraction has a diameter ranging from at least about 1 μm, about 2 μm, about 3 μm, about 4 μm, about 5 μm, about 6 μm, about 7 μm, about 8 μm, or about 9 μm. In some embodiments, the enucleated cell of the enucleated cell fraction has a diameter ranging from at most about 2 μm, about 3 μm, about 4 μm, about 5 μm, about 6 μm, about 7 μm, about 8 μm, about 9 μm, or about 10 μm. In some embodiments, the enucleated cell of the enucleated cell fraction has a diameter of about 8 μm.

In some embodiments, the methods for cell processing further include generating the density gradient by centrifuging a density gradient media with acceleration spanning over at least about 1 minute (min), at least about 5 min, at least about 10 min, at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min. In some embodiments, the methods for cell processing further include generating the density gradient by centrifuging a polysaccharide with acceleration spanning over at least about 1 min, at least about 5 min, at least about 10 min, at least about 15 min, at least about 20 min, at least about 25 min, at least about 30 min, at least about 35 min, at least about 40 min, at least about 45 min, at least about 50 min, at least about 55 min, at least about 60 min, at least about 90 min, or at least about 120 min. In some embodiments, the methods for cell processing further include generating the density gradient by centrifuging a polysaccharide with acceleration spanning over at least about 10 min, at least about 20 min, at least about 30 min, at least about 40 min, or at least about 50 min. In some embodiments, the methods for cell processing further include generating the density gradient by centrifuging a polysaccharide with acceleration spanning over at least about 30 min.

In some embodiments, the methods of enucleating a portion of the nucleated cells to produce an enucleated cell fraction using continuous flow centrifugation for cell processing further include generating the density gradient by centrifuging a polysaccharide with minimal deceleration. In some embodiments, the density gradient comprising centrifuging a polysaccharide at a maximum centrifugal force of between about 20,000 relative centrifugal force (RCF) to about 250,000 RCF. In some embodiments, the density gradient comprising centrifuging a polysaccharide at a maximum centrifugal force of between about 20,000 RCF to about 30,000 RCF, about 20,000 RCF to about 40,000 RCF, about 20,000 RCF to about 50,000 RCF, about 20,000 RCF to about 60,000 RCF, about 20,000 RCF to about 70,000 RCF, about 20,000 RCF to about 80,000 RCF, about 20,000 RCF to about 100,000 RCF, about 20,000 RCF to about 120,000 RCF, about 20,000 RCF to about 150,000 RCF, about 20,000 RCF to about 200,000 RCF, about 20,000 RCF to about 250,000 RCF, about 30,000 RCF to about 40,000 RCF, about 30,000 RCF to about 50,000 RCF, about 30,000 RCF to about 60,000 RCF, about 30,000 RCF to about 70,000 RCF, about 30,000 RCF to about 80,000 RCF, about 30,000 RCF to about 100,000 RCF, about 30,000 RCF to about 120,000 RCF, about 30,000 RCF to about 150,000 RCF, about 30,000 RCF to about 200,000 RCF, about 30,000 RCF to about 250,000 RCF, about 40,000 RCF to about 50,000 RCF, about 40,000 RCF to about 60,000 RCF, about 40,000 RCF to about 70,000 RCF, about 40,000 RCF to about 80,000 RCF, about 40,000 RCF to about 100,000 RCF, about 40,000 RCF to about 120,000 RCF, about 40,000 RCF to about 150,000 RCF, about 40,000 RCF to about 200,000 RCF, about 40,000 RCF to about 250,000 RCF, about 50,000 RCF to about 60,000 RCF, about 50,000 RCF to about 70,000 RCF, about 50,000 RCF to about 80,000 RCF, about 50,000 RCF to about 100,000 RCF, about 50,000 RCF to about 120,000 RCF, about 50,000 RCF to about 150,000 RCF, about 50,000 RCF to about 200,000 RCF, about 50,000 RCF to about 250,000 RCF, about 60,000 RCF to about 70,000 RCF, about 60,000 RCF to about 80,000 RCF, about 60,000 RCF to about 100,000 RCF, about 60,000 RCF to about 120,000 RCF, about 60,000 RCF to about 150,000 RCF, about 60,000 RCF to about 200,000 RCF, about 60,000 RCF to about 250,000 RCF, about 70,000 RCF to about 80,000 RCF, about 70,000 RCF to about 100,000 RCF, about 70,000 RCF to about 120,000 RCF, about 70,000 RCF to about 150,000 RCF, about 70,000 RCF to about 200,000 RCF, about 70,000 RCF to about 250,000 RCF, about 80,000 RCF to about 100,000 RCF, about 80,000 RCF to about 120,000 RCF, about 80,000 RCF to about 150,000 RCF, about 80,000 RCF to about 200,000 RCF, about 80,000 RCF to about 250,000 RCF, about 100,000 RCF to about 120,000 RCF, about 100,000 RCF to about 150,000 RCF, about 100,000 RCF to about 200,000 RCF, about 100,000 RCF to about 250,000 RCF, about 120,000 RCF to about 150,000 RCF, about 120,000 RCF to about 200,000 RCF, about 120,000 RCF to about 250,000 RCF, about 150,000 RCF to about 200,000 RCF, about 150,000 RCF to about 250,000 RCF, or about 200,000 RCF to about 250,000 RCF. In some embodiments, the density gradient comprising centrifuging a polysaccharide at a maximum centrifugal force of between about 20,000 RCF, about 30,000 RCF, about 40,000 RCF, about 50,000 RCF, about 60,000 RCF, about 70,000 RCF, about 80,000 RCF, about 100,000 RCF, about 120,000 RCF, about 150,000 RCF, about 200,000 RCF, or about 250,000 RCF. In some embodiments, the density gradient comprising centrifuging a polysaccharide at a maximum centrifugal force of between about 20,000 RCF, about 30,000 RCF, about 40,000 RCF, about 50,000 RCF, about 60,000 RCF, about 70,000 RCF, about 80,000 RCF, about 100,000 RCF, about 120,000 RCF, about 150,000 RCF, or about 200,000 RCF. In some embodiments, the density gradient comprising centrifuging a polysaccharide at a maximum centrifugal force at most about 30,000 RCF, about 40,000 RCF, about 50,000 RCF, about 60,000 RCF, about 70,000 RCF, about 80,000 RCF, about 100,000 RCF, about 120,000 RCF, about 150,000 RCF, about 200,000 RCF, or about 250,000 RCF.

### Modifying Cells of the Disclosure

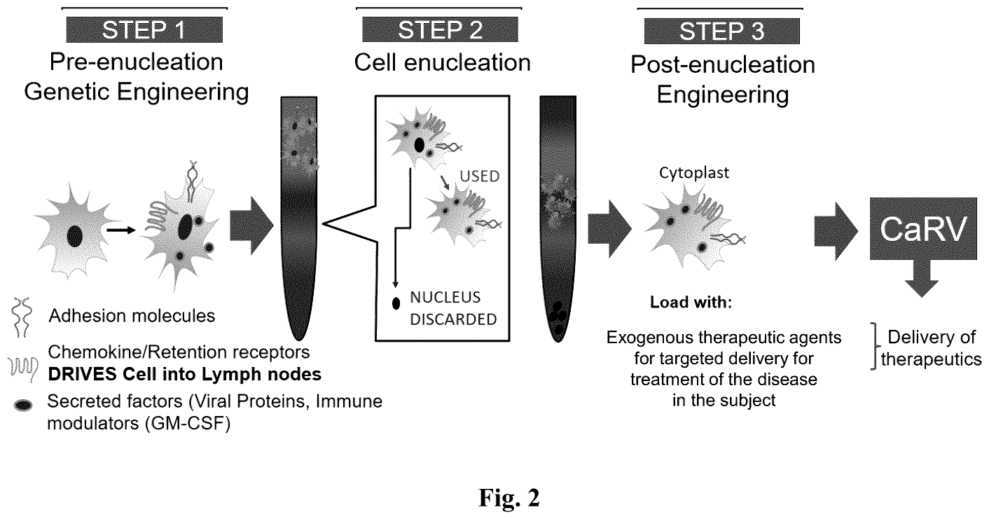

As shown in FIG. 2, a nucleated (“parent”) cell may be engineered prior to enucleation to express one or more exogenous agents, or after enucleation, or a combination thereof. In some embodiments, the one or more exogenous biomolecule comprises a targeting moiety, a transmembrane moiety, a biomolecular suicide switch, or a therapeutic agent, or a combination thereof. In some embodiments, the targeting moiety comprises an adhesion molecule, chemokine or retention receptors or both. In some embodiments, the targeting moiety is engineered to target a target tissue, cell or environment disclosed herein (e.g., the lymph tissue in a subject). In addition, or alternatively, the resulting enucleated cell is engineered to express and, in some cases, to secrete the therapeutic agent. In some embodiments, the therapeutic agent comprises an antibody or an antigen-binding fragment thereof (e.g., single-domain antibody). In some embodiments, the enucleated cell may be administered to a subject in need thereof to treat a disease or a condition in the subject.

Various methods may be used to introduce a biomolecule (e.g., the therapeutic agent, transmembrane moiety, immune-evading moiety, and/or targeting moiety described herein) into the parent cell or the enucleated cell described herein. Non-limiting examples of methods that may be used to introduce a biomolecule into the parent cell or the enucleated cell include: liposome mediated transfer, an adenovirus, an adeno-associated virus, a herpes virus, a retroviral based vector, a lentiviral vector, electroporation, microinjection, lipofection, transfection, calcium phosphate transfection, dendrimer-based transfection, cationic polymer transfection, cell squeezing, sonoporation, optical transfection, impalection, hydrodynamic delivery, magnetofection, nanoparticle transfection, or combinations thereof. In some aspects of any of the compositions and methods provided herein, a therapeutic agent, a virus, an antibody, or a nanoparticle may be introduced into the enucleated cells.

In some embodiments, the enucleated cell is preserved via cryopreservation, cryohibernation, or lyophilization. Cryopreservation comprises freezing the enucleated cell, while cryohibemation comprises storing the enucleated cell at a temperature that is below room temperature but without freezing the enucleated cell. In some embodiments, the enucleated cell is lyophilized. In some embodiments, the lyophilized enucleated cell can be reconstituted, and the reconstituted enucleated cell exhibits comparable viability to the enucleated cell that has not been lyophilized. In some embodiments, the lyophilization comprises components: freezing the cell; subjecting the cell to drying under a very low pressure (e.g., <3000 mTorr) using vacuum. The drying component can lead to sublimation and dehydrate the cell while maintaining cellular viability and biologic function. In some embodiments, the freezing phase comprises balancing the duration and temperature of the freezing to for maintaining cell viability and stability, appropriate crystal formation, and the speed of reconstitution. The triple point of a substance is the temperature and the pressure at which the sublimation curve, fusion curve, and vaporization curve meet. Achievement of the triple point which varies for different substances ensures that sublimation rather than melting will occur in the following drying steps. To facilitate faster and more efficient freeze-drying, larger ice crystals are preferred, because they form a network within the product that promotes faster removal of water vapor during sublimation. To produce larger crystals, the product should be frozen slowly or the temperature can be cycled up and down in a process called annealing. Fresh or frozen living tissue or cells do not have a single homogeneous melting point (eutectic point) and consequently the freezing stage of the material (cells or tissue) is cooled below its triple point which represents the temperature and pressure at which the solid, liquid and gas phases of the material can coexist. Living cells do have a critical point on a phase diagram at which both the liquid and the gas phase of an object or substance have the same density and are therefore indistinguishable. The product critical point temperature must be maintained to prevent melt-back or cake collapse occurring during primary and secondary drying which reflects incomplete sublimation. In the case of substances where preservation of structure is required like living cells, large ice crystals may be detrimental and may break the cell walls which can result in increasingly poor texture and loss of nutritive content. In this case, the freezing should be done rapidly, in order to lower the material to below its critical point quickly, thus avoiding the formation of large ice crystals. The freezing temperatures for cells or tissue can vary but ranges in general between −50° C. (−58° F.) and −80° C. (−112° F.).

During the drying phase, the ambient pressure is lowered to the range of a few millibars, and then heat is supplied by conduction or radiation to the material for the ice to sublime. The amount of heat necessary can be calculated using the sublimating molecules' latent heat of sublimation. In this initial drying phase, about 95% of the water in the material or substance is sublimated. This phase is often slow and can even last for several days depending on the substance and technology employed but if too much heat is added to quickly the material's structure could be altered. In this phase, pressure is controlled through the application of a partial vacuum. The vacuum speeds up the sublimation, making it useful as a deliberate drying process. A cold condenser chamber and/or condenser plates are used as a surface(s) for the water vapor to re-liquify and solidify on. It is important to note that in this range of pressure, the heat cannot be provided by a convection effect because of the low air density. The drying phase also aims to remove remaining unfrozen water molecules since the ice induced with freezing should be removed during the primary drying phase. This part of the freeze-drying process is governed by the material's adsorption isotherms. In this phase, the temperature is raised higher than in the primary drying phase and can even be above 0° C. (32° F.), to break any physico-chemical interactions that have formed between the water molecules and the frozen material. Usually, during this phase the pressure is also lowered in this stage to encourage desorption. However, there are products that benefit from increased pressure as well. After the freeze-drying process is complete, the vacuum is usually broken with an inert gas, such as nitrogen, before the material is sealed. At the end of the operation, the residual water content in the product is extremely low and should range from <1% to 4% of the original concentration.

In some embodiments, the lyophilization of the enucleated cell comprises the use of lyoprotectants for retaining cell viability and biologic function. Lyoprotectant comprises addition of reagents, salts, or additives that protects cell during the desiccation process. Common lyoprotectants include trehalose, DMSO, methylcellulose, sucrose, antioxidants, human or animal serum proteins, and cellular stress proteins. Additionally, methods for increasing the transport of lyoprotectants inside the cells in suspension can be utilized as a way of improving the viability and function of cells after lyophilization. These methods include electroporation, addition of reagents that enhance intracellular transport, genetic modification of cells to upregulate the expression of pores on cell membranes, and mechanical microfluidic devices that partially disrupt cell membrane integrity and potentially promote intracellular transport of lyoprotectants.

In some embodiments, the nucleated cell described herein can be modified to express a targeting moiety (e.g., an antibody or antigen binding fragment thereof), a therapeutic agent, a transmembrane moiety, a heterologous gene product, or a combination thereof. In some embodiments, the nucleated cell can be modified to express at least one heterologous polynucleotide, where the at least one heterologous polynucleotide encodes a targeting moiety, a therapeutic agent, a transmembrane moiety, heterologous gene product, or a combination thereof.

In one aspect, disclosed herein are methods for modifying a cell by introducing at least one heterologous polynucleotide into the cell. In some embodiments, the heterologous polynucleotide encodes promoter, a heterologous gene product, or a combination thereof. In some embodiments, the methods comprise providing a composition comprising a first subset of nucleated cells and enucleated cells derived from a second subset of the nucleated cells. In some embodiments, the first subset of the nucleated cells comprises a heterologous polynucleotide encoding a heterologous gene product. In some embodiments, the methods comprise expressing the heterologous gene product thereby inducing cell death of at least one nucleated cell of the first subset of the nucleated cells. The heterologous polynucleotide can be introduced into any types of cells that could be enucleated, for instance, but not limited to, hTERT-immobilized Mesenchymal stem cells, by transfection of plasmids, transposons, zinc finger nucleases (ZFNs), transcription activator-like effector nucleases (TALENs), CRISPR-Cas technologies, viral transduction, etc.

In some embodiments, the heterologous polynucleotide comprises a promoter. In some embodiments, the promoter comprises an inducible promoter. In some embodiments, the promoter should be compatible with mammalian gene expression, provide rapid, strong gene expression, only in the presence of its induction stimulus. Associated suicide genes may include, but are not limited to, caspases, “eat me” signals, DNA crosslinkers, death inducing synthetic NOTCH receptors, toxins, and apoptosis/autophagy/entosis/necrosis/necroptosis/ferroptosis induction agents. Each promoter has its own activation protocol in terms of temperature, incubation time, light wavelength, inducer concentrations, etc.

In some embodiments, the inducible promoter is hypothermic. In some embodiments, the inducible promoter is induced by contacting the nucleated cells with a temperature that is below about 40° C. In some embodiments, the inducible promoter is induced by contacting the nucleated cells with a temperature that is below about 39° C. In some embodiments, the inducible promoter is induced by contacting the nucleated cells with a temperature that is below about 38° C. In some embodiments, the inducible promoter is induced by contacting the nucleated cells with a temperature that is below about 37° C. In some embodiments, the inducible promoter is induced by contacting the nucleated cells with a temperature that is below about 36° C. In some embodiments, the inducible promoter is induced by contacting the nucleated cells with a temperature that is below about 35° C. In some embodiments, examples of the inducible promoter include, but not limited to, comprises dsrA or CIRP.

In some embodiments, the inducible promoter is hyperthermic. In some embodiments, the inducible promoter is induced by contacting the nucleated cells with a temperature that is above 35° C. In some embodiments, the inducible promoter is induced by contacting the nucleated cells with a temperature that is above 36° C. In some embodiments, the inducible promoter is induced by contacting the nucleated cells with a temperature that is above 37° C. In some embodiments, the inducible promoter is induced by contacting the nucleated cells with a temperature that is above 38° C. In some embodiments, the inducible promoter is induced by contacting the nucleated cells with a temperature that is above 39° C. In some embodiments, the inducible promoter is induced by contacting the nucleated cells with a temperature that is above 40° C. In some embodiments, examples of the inducible promoter include, but not limited to, heat shock protein 70 (HSP70), heat shock protein 90 (HSP90), growth arrest- and DNA damage-inducible gene 153 (GADD153), multidrug resistance mutation 1 (MDR1), or cytomegalovirus (HSE-CMV).

In some embodiments, the inducible promoter is induced by contacting the nucleated cells with a molecule. In some embodiments, examples of the molecule include, but not limited to, rtTA, TRE, TetR, Cumate, Rapamycin, Abscisic acid, IPTG, or Methallothionein.

In some embodiments, the inducible promoter is induced by contacting the nucleated cells with light. In some embodiments, examples of the inducible promoter include, but not limited to, CIB1-CRY2 or GAL4-VVD.

In some embodiments, the inducible promoter is induced by contacting with the nucleated cells with a hormone. In some embodiments, examples of the inducible promoter include, but not limited to, Estradiol-Gal4.

In some embodiments, the promoter comprises a constitutively active promoter. The promoter will be continually active, but suicide will be induced under certain circumstances. In some embodiments, the constitutively active promoter is configured to activate transcription of the heterologous polynucleotide under conditions sufficient to express the heterologous gene product. In some embodiments, examples of the heterologous gene product include, but not limited to, herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase. In some embodiments, examples of the heterologous gene product include, but not limited to, FKBP or a caspase. In some embodiments, an example of heterologous gene product includes, but not limited to, an antigen. In some embodiments, the heterologous polynucleotide is integrated into chromosome of the nucleated cells. In some embodiments, an example of the heterologous polynucleotide includes, but not limited to, a vector.

In some embodiments, the enucleated cell described herein can be cryopreserved, cryohibemated, lyophilized, or a combination thereof. In some embodiments, the cryopreserved enucleated cell, following thawing, the enucleated cell is as viable as an otherwise comparable enucleated cell that is not cryopreserved. In some embodiments, the lyophilized enucleated cell is as viable as an otherwise comparable enucleated cell that is not lyophilized. In some embodiments, the cryohibemated enucleated cell is as viable as an otherwise comparable enucleated cell that is not cryohibernated.

### Method of Treatment

Disclosed herein, in some embodiments, are methods of using the enucleated cell, the composition, or the pharmaceutical composition described herein for treating a disease or condition. In some embodiments, the methods include treating the disease or condition of the subject by administering a composition described herein (e.g., a pharmaceutical composition containing enucleated cells engineered to express a therapeutic agent) to the subject. In some embodiments, the enucleated cells disclosed herein may be loaded, transfected or transduced with a therapeutic agent disclosed herein or any existing therapeutic agent, formulated in a pharmaceutical formulation, and the pharmaceutical formulation may be delivered to a subject according to various embodiments herein. The pharmaceutical formulations disclosed herein increases biodistribution and/or homing of the therapeutic agent to target cells or tissues in vivo, as compared to administering the therapeutic agent that is not encapsulated by or expressed in the enucleated cells disclosed herein.

The present disclosure also provides methods for the use of enucleated cells (natural or enucleated) as fusion partners to other cells (therapeutic or natural) to enhance and/or transfer biomolecules described herein, such as for example, a therapeutic agent. In some embodiments, the biomolecules include, DNA/genes, RNA (mRNA, shRNA, siRNA, miRNA), nanoparticles, peptides, proteins, and plasmids, bacteria, viruses, small molecule drugs, ions, cytokines, growth factors, and hormones. In some embodiments, the enucleated cell is engineered to express a fusogenic moiety. The fusogenic moiety can be any biomolecule (e.g., sugar, lipid, or protein) that promotes fusion of the membrane. In some embodiments, the fusogenic moiety is a fusogenic protein. A fusogenic protein allows the enucleated cell expressing the fusogenic protein to fuse with a target cell. In some embodiments, the fusogenic protein facilitates the merging of an enucleated cell expressing the fusogenic protein with a target cell, allowing the contents of the enucleated cell to enter into the target cell. In some embodiments, the fusogenic protein is heterotypic such as viral classes I-III or HAP2/GCS1 or SNARE. In some embodiments, the fusogenic protein is homoleptic such as EFF-1/AFF-1. Other non-limiting examples of the fusogenic protein is Izumol or Syncytin. In some embodiments, the fusogenic protein is a viral protein. In some embodiments, the fusogenic protein from a virus is VSV-g, hERV-W-ENV (Syncytin), or MV-Ed-F+MV-Ed-H (Hemagglutinin). Unlike nucleated cells, the fusion of enucleated cells to the same or another cell type of similar or different origin generates a unique cell hybrid that lacks problematic nuclear transfer, while maintaining desirable therapeutic attributes including, but not limited to, cell surface proteins, signal transduction molecules, secreted proteins, and epigenetic changes.

### Subject

The methods disclosed herein, in some embodiments, comprise administering or delivering a composition (e.g., pharmaceutical composition) to a subject. In some embodiments, the subject has a disease, disorder, or condition, (e.g., cancer, idiopathic pulmonary fibrosis). In some embodiments, the subject is a mammal. In some embodiments, the mammal is a human. In some embodiments, the subject is an adolescent, an adult, or an elderly subject. In some embodiments, the human subject is at least 18 years of age. In some embodiments, the human subject is age 18 to about 55 years. In some embodiments, the human subject is more than 55 years old. In some embodiments, the subject is age 18 to about 65 years. In some embodiments, the subject is more than 65 years old. In some embodiments, the subject is a female. In some embodiments, the subject is a male. In some embodiments, the subject is immunocompromised, or at increased risk for being immunocompromised.

### Disease or Condition

Provided herein are methods of treating a disease or a condition in a subject by administering a composition described herein to the subject. In some embodiments, administration is by any suitable mode of administration, including systemic administration (e.g., intravenous, inhalation, etc.). In some embodiments, the subject is human. In some embodiments, the disease or the condition comprises an infection (e.g., human immunodeficiency virus (HIV)-infection, Chagas disease, tuberculosis), a neurological disease (e.g., Parkinson's Disease, Huntington's Disease, Alzheimer's Disease) an autoimmune disease (e.g., diabetes, Crohn's disease, multiple sclerosis, sickle cell anemia), a cardiovascular disease (e.g., acute myocardial infarction, heart failure, refractory angina), a ophthalmologic disease, a skeletal disease, a metabolic disease (e.g., phenylketonuria, glycogen storage deficiency type 1A, Gaucher disease), an inflammatory disease (e.g., cancer, inflammatory bowel disease), or a disease caused by external pathogen or toxin in a subject. In some embodiments, the disease or the condition comprises idiopathic pulmonary fibrosis. In some embodiments, the subject is in need of, or has been determined to be in need of, such an enucleated cell treatment.

In some embodiments, the cancer may be lung cancer, including non-small cell lung cancer (NSCLC), small cell lung cancer (SCLC), or any other lung cancer type. For example, the lung cancer may include adenocarcinoma, squamous carcinoma, large cell (undifferentiated) carcinoma, large cell neuroendocrine carcinoma, adenosquamous carcinoma, sarcomatoid carcinoma, lung carcinoid tumor, or adenoid cystic carcinoma. Other non-limiting example of lung cancer includes lymphoma, sarcoma, benign lung tumor, or hamartoma. In some embodiments, the cancer is metastatic cancer. In some embodiments, the cancer metastasized to the lung from a different tissue or source. For example, the metastatic cancer that may be found in the lung may include breast cancer, colon cancer, prostate cancer, sarcoma, bladder cancer, neuroblastoma, and Wilm's tumor.

In some embodiments, the enucleated cell described herein comprises a targeting moiety described herein for binding to an epitope expressed by a cancer cell or an epitope associated with a tumor microenvironment. In some embodiments, the targeting moiety comprises an antibody or antigen-binding fragment thereof described herein. In some embodiments, the antibody or antigen-binding fragment thereof comprises a single-domain antibody. In some embodiments, the antibody or antigen-binding fragment binds to an epitope expressed by a cancer cell or an epitope associated with a tumor microenvironment. In some embodiments, the binding of the targeting moiety (e.g., the antibody or the antigen-binding fragment thereof) to the epitope provides a therapeutic effect to treat cancer in a subject. In some embodiments, the binding of the targeting moiety (e.g., antibody or the antigen-binding fragment thereof) to the epitope recruits immune cells to activate immune response against the cancer.

In some embodiments, described herein are enucleated cells and methods of using these enucleated cells to treat a disease or condition associated with abnormal vasculature in a subject. Abnormal vasculature can be associated with disease or condition such as inflammation and cancer (e.g., any one of the cancers described herein). In some embodiments, the enucleated cells described herein, when contacted with the abnormal vasculature, increases the normalization of the abnormal vasculature, where the adhesion between endothelial cells is increased to prevent leakage of intravascular factors out of the vasculature. In some embodiments, the normalization of the abnormal vasculature includes decreasing of damages such as cell dead of the endothelial cells of the vasculature. In some embodiments, the normalization of the abnormal vasculature includes angiogenesis of immature or leaky vessels. In some embodiments, the normalization exerted by the enucleated cells can include normalization of blood vessel, lymphatic vessel, or a combination thereof.

In some embodiments, the disease or condition may be caused by a pathogen. In some embodiments, the enucleated cell described herein comprises an antibody or an antigen-binding fragment thereof or single-domain antibody that binds to an epitope expressed by the pathogen or an epitope associated with a microenvironment associated with the pathogen. In some cases, the binding of the antibody or the antigen-binding fragment thereof or single-domain antibody to the epitope confers therapeutic property against the pathogen. In some embodiments, the binding of the antibody or the antigen-binding fragment thereof or single-domain antibody to the epitope recruits immune cells to activate immune response to confer therapeutic property against the pathogen. For example, the disease or condition may be caused by virus, bacterium, fungus, parasite, or molecule resulted from detoxification. In some embodiments, the pathogens: may be disseminated or transmitted from person to person; result in high mortality rates and have the potential for major public health impact; may cause public panic and social disruption; and require special action for public health preparedness. Example of these pathogens may include Anthrax (Bacillus anthracis), Botulism (Clostridium botulinum toxin), Plague (Yersinia pestis), Smallpox (variola major), Tularemia (Francisella tularensis), or Viral hemorrhagic fevers, including Filoviruses (Ebola, Marburg) and Arenaviruses (Lassa, Machupo).

In some embodiments, the pathogen: may be disseminated; resulting in moderate morbidity rates and low mortality rates; and require specific enhancements of diagnostic capacity and enhanced disease surveillance. Example of these pathogens may include Brucellosis (Brucella species), Epsilon toxin of Clostridium perfringens, Food safety threats (e.g., Salmonella species, Escherichia coli 0157:H7, or Shigella), Glanders (Burkholderia mallei), Melioidosis (Burkholderia pseudomallei), Psittacosis (Chlamydia psittaci), Q fever (Coxiella bumetii), Ricin toxin from Ricinus communis (castor beans), Staphylococcal enterotoxin B, Typhus fever (Rickettsia prowazekii), Viral encephalitis (alphaviruses, such as eastern equine encephalitis, Venezuelan equine encephalitis, and western equine encephalitis), or Water safety threats (e.g., Vibrio cholerae and Cryptosporidium parvum).

In some embodiments, the pathogen may include emerging pathogen that has a high potential for mortality and morbidity, but the extend of which is not fully understood. Non-limiting examples of these pathogens may include Nipah virus and hantavirus.

The enucleated cells described herein, or the composition containing such enucleated cells (referred to in this section as “composition”) may be administered to a subject in a suitable dose, mod of administration, and frequency, which depends on the intended effect.

In some embodiments, the composition is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60,70, 80, 90, 100 times) during a period of time.

In some embodiments, the composition is administered in a therapeutically-effective amount by various forms and routes including, for example, oral, or topical administration. In some embodiments, a composition may be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

In some embodiments, the composition is administered by intravenous infusion. In some embodiments, the composition is administered by slow continuous infusion over a long period, such as more than 24 hours. In some embodiments, the composition is administered as an intravenous injection or a short infusion.

A composition may be administered in a local manner, for example, via injection of the agent directly into an organ, optionally in a depot or sustained release formulation or implant. A composition may be provided in the form of a rapid release formulation, in the form of an extended release formulation, or in the form of an intermediate release formulation. A rapid release form may provide an immediate release. An extended release formulation may provide a controlled release or a sustained delayed release. In some embodiments, a pump may be used for delivery of the composition. In some embodiments, a pen delivery device may be used, for example, for subcutaneous delivery of a composition of the disclosure. The composition provided herein may be administered in conjunction with other therapies, for example, an antiviral therapy, a chemotherapy, an antibiotic, a cell therapy, a cytokine therapy, or an anti-inflammatory agent.

The compositions (e.g., enucleated cells or pharmaceutical composition comprising the enucleated cell described herein) may be administered before, during, or after the occurrence of a disease or condition, and the timing of administering the composition containing a therapeutic agent may vary. In some cases, the composition may be used as a prophylactic and may be administered continuously to subjects (e.g., the subject for immunization or the subject for treatment) with a susceptibility to a coronavirus or a propensity to a condition or disease associated with a coronavirus. Prophylactic administration may lessen a likelihood of the occurrence of the infection, disease or condition, or may reduce the severity of the infection, disease or condition.

The composition may be administered to a subject before the onset of the symptoms. The composition may be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen level. A composition may be administered after (e.g., as soon as is practicable after) the onset of a disease or condition is detected or suspected. A composition may be administered after (e.g., as soon as is practicable after) a potential exposure to a coronavirus, for example, after a subject (e.g., the subject for immunization or the subject for treatment) has contact with an infected subject or learns they had contact with an infected subject that may be contagious.

Actual dosage levels of an agent of the disclosure (e.g., antibody or antigen-binding fragment thereof, or therapeutic agent) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions employed herein, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

A composition described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

A dose may be based on the amount of the agent per kilogram of body weight of a subject (e.g., the subject for immunization or the subject for treatment). A dose of an agent (e.g., antibody) is in the range of 10-3000 mg/kg, e.g., 100-2000 mg/kg, e.g., 300-500 mg/kg/day for 1-10 or 1-5 days; e.g., 400 mg/kg/day for 3-6 days; e.g., 1 g/kg/d for 2-3 days In some embodiments, a dose may be based on the number of the enucleated cells per kilogram of body weight of a subject. In some embodiments, a dose may be is administered in a dosage amount of between about 1,000 cells/kg body weight to about 1,000,000,000,000 cells/kg body weight. In some embodiments, a dose may be is administered in a dosage amount of between about 1000 cells/kg body weight to about 1,000,000,000,000 cells/kg body weight, about 1,000 cells/kg body weight to about 10,000 cells/kg body weight, about 1,000 cells/kg body weight to about 100,000 cells/kg body weight, about 1,000 cells/kg body weight to about 1,000,000 cells/kg body weight, about 1,000 cells/kg body weight to about 10,000,000 cells/kg body weight, about 1,000 cells/kg body weight to about 100,000,000 cells/kg body weight, about 1,000 cells/kg body weight to about 1,000,000,000 cells/kg body weight, about 1,000 cells/kg body weight to about 10,000,000,000 cells/kg body weight, about 1,000 cells/kg body weight to about 100,000,000,000 cells/kg body weight, about 1,000 cells/kg body weight to about 1,000,000,000,000 cells/kg body weight, about 10,000 cells/kg body weight to about 100,000 cells/kg body weight, about 10,000 cells/kg body weight to about 1,000,000 cells/kg body weight, about 10,000 cells/kg body weight to about 10,000,000 cells/kg body weight, about 10,000 cells/kg body weight to about 100,000,000 cells/kg body weight, about 10,000 cells/kg body weight to about 1,000,000,000 cells/kg body weight, about 10,000 cells/kg body weight to about 10,000,000,000 cells/kg body weight, about 10,000 cells/kg body weight to about 100,000,000,000 cells/kg body weight, about 10,000 cells/kg body weight to about 1,000,000,000,000 cells/kg body weight, about 100,000 cells/kg body weight to about 1,000,000 cells/kg body weight, about 100,000 cells/kg body weight to about 10,000,000 cells/kg body weight, about 100,000 cells/kg body weight to about 100,000,000 cells/kg body weight, about 100,000 cells/kg body weight to about 1,000,000,000 cells/kg body weight, about 100,000 cells/kg body weight to about 10,000,000,000 cells/kg body weight, about 100,000 cells/kg body weight to about 100,000,000,000 cells/kg body weight, about 100,000 cells/kg body weight to about 1,000,000,000,000 cells/kg body weight, about 1,000,000 cells/kg body weight to about 10,000,000 cells/kg body weight, about 1,000,000 cells/kg body weight to about 100,000,000 cells/kg body weight, about 1,000,000 cells/kg body weight to about 1,000,000,000 cells/kg body weight, about 1,000,000 cells/kg body weight to about 10,000,000,000 cells/kg body weight, about 1,000,000 cells/kg body weight to about 100,000,000,000 cells/kg body weight, about 1,000,000 cells/kg body weight to about 1,000,000,000,000 cells/kg body weight, about 10,000,000 cells/kg body weight to about 100,000,000 cells/kg body weight, about 10,000,000 cells/kg body weight to about 1,000,000,000 cells/kg body weight, about 10,000,000 cells/kg body weight to about 10,000,000,000 cells/kg body weight, about 10,000,000 cells/kg body weight to about 100,000,000,000 cells/kg body weight, about 10,000,000 cells/kg body weight to about 1,000,000,000,000 cells/kg body weight, about 100,000,000 cells/kg body weight to about 1,000,000,000 cells/kg body weight, about 100,000,000 cells/kg body weight to about 10,000,000,000 cells/kg body weight, about 100,000,000 cells/kg body weight to about 100,000,000,000 cells/kg body weight, about 100,000,000 cells/kg body weight to about 1,000,000,000,000 cells/kg body weight, about 1,000,000,000 cells/kg body weight to about 10,000,000,000 cells/kg body weight, about 1,000,000,000 cells/kg body weight to about 100,000,000,000 cells/kg body weight, about 1,000,000,000 cells/kg body weight to about 1,000,000,000,000 cells/kg body weight, about 10,000,000,000 cells/kg body weight to about 100,000,000,000 cells/kg body weight, about 10,000,000,000 cells/kg body weight to about 1,000,000,000,000 cells/kg body weight, or about 100,000,000,000 cells/kg body weight to about 1,000,000,000,000 cells/kg body weight. In some embodiments, a dose may be is administered in a dosage amount of between about 1000 cells/kg body weight to about 1000000000000 cells/kg body weight, about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, a dose may be is administered in a dosage amount of between about 1000 cells/kg body weight to about 1000000000000 cells/kg body weight, at least about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, or about 100,000,000,000 cells/kg body weight. In some embodiments, a dose may be is administered in a dosage amount of between about 1000 cells/kg body weight to about 1000000000000 cells/kg body weight, at most about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, the cell without the nucleus is administered to the subject twice within at least an hour, 2 hours, 4 hours, 6 hours, 8 hours, 12 hours, 1 day, 2 days, a week, 2 weeks, 3 weeks, a month, 2 months, 3 months, 4 months, 5 months, 6 months, 7 months, 8 months, 9 months, 10 months, 11 months, a year, 2 years, 3 years, or 4 years.

Disclosed herein are methods of repeatedly administering the composition or the pharmaceutical composition to the subject in need thereof. In some embodiments, the first administrations of the composition or pharmaceutical composition comprising the enucleated cell normalizes blood vessel or lymphatic vessel. In some embodiments, the composition or pharmaceutical composition comprising the same enucleated cell can be subsequently administered to the subject for: maintaining the normalization of the blood vessel or lymphatic vessel; and delivering the exogenous agent for treating a disease or condition described herein.

### Kits

Disclosed herein, in some aspects, are kits for using the compositions described herein. In some embodiments, the kits disclosed herein may be used to treat a disease or condition in a subject. In some embodiments, the kits comprise an assemblage of materials or components apart from the composition. In some embodiments, the kit comprises nucleated cell described herein (e.g., nucleated cell engineered to express a targeting moiety (e.g., antibody or antigen-binding fragment thereof), a therapeutic agent, a transmembrane moiety, an immune-evading moiety, a heterologous gene produce, or a combination thereof. In some embodiments, the kit can include enucleated cells obtained from the nucleated cells. In some embodiments, the kit can include a mixed population of nucleated cells and enucleated cells obtained from the nucleated cells. In some embodiments, the kit can include a substantially pure population of enucleated cells. In some embodiments, the kit comprises nucleated cells, enucleated cells, or a combination thereof suspended in at least one density gradient.

In some embodiments, the kit comprises a pharmaceutical formulation disclosed herein, comprising the enucleated cells engineered to express (and in some cases secrete) a targeting moiety (e.g., antibody or antigen-binding fragment thereof), a therapeutic agent, a transmembrane moiety, an immune-evading moiety, a heterologous gene produce, or a combination thereof. In some embodiments, the enucleated cell expresses or secretes the therapeutic agent such as an immune checkpoint molecule or an immune checkpoint inhibitor for treating a disease or condition in a subject. In some embodiments, the enucleated cells are further engineered to express a targeting moiety, such as a chemokine receptor, an integrin signaling molecule or an antibody or antigen-binding fragment thereof that enables the enucleated cells to efficiently migrate to the target tissue in a subject, once administered. In some embodiments, the kits further comprise an additional therapeutic agent, such as those disclosed herein. In some embodiments, the kit further comprises instructions for administering the pharmaceutical formulation and/or additional therapeutic agent to the subject to treat a disease or a condition in the subject such as cancer. In some embodiments, the cancer comprises cancer of the lung tissue.

In some embodiments, the cancer is lung cancer. In some embodiments, the kit comprises components for purifying enucleated cells from nucleated cells or other cellular debris. For example, the kit can include filter membranes with different pore sizes to isolate and purify the enucleated cells. In some embodiments, the kit comprises components for staining and selecting for enucleated cells. For example, the kit can include florescent dye for staining nucleus, where the nucleated cell can eb stained and selectively removed, leaving a population of enucleated cells. In some embodiments, the kit comprises components for inducing cell death of the nucleated cell. For example, the kit can include molecules for inducing expression of the heterologous gene product described herein for inducing cell death of nucleated cell.

In some embodiments, the kit described herein comprises components for selecting for a homogenous population of the enucleated cells. In some embodiments, the kit described herein comprises components for selecting for a heterogenous population of the enucleated cells. In some embodiments, the kit comprises the components for assaying the number of units of a biomolecule (e.g., a therapeutic agent) synthesized, and/or released or expressed on the surface by the enucleated cell. In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the composition to a subject in need thereof. In some embodiments, the kit comprises instructions for further engineering the composition to express a biomolecule (e.g., a therapeutic agent). In some embodiments, the kit comprises instructions thawing or otherwise restoring biological activity of the composition, which may have been cryopreserved, lyophilized, or cryo-hibernated during storage or transportation. In some embodiments, the kit comprises instructions for measure viability of the restored compositions, to ensure efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components may be contained in suitable packaging material(s).

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease may be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level.

The terms “individual” or “subject” are used interchangeably and encompass mammals. Non-limiting examples of mammal include any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The mammal may be a human. The term “animal” as used herein comprises human beings and non-human animals. In one embodiment, a “non-human animal” is a mammal, for example a rodent such as rat or a mouse. A “patient,” as used herein refers to a subject that has, or has been diagnosed with, a disease or a condition described herein.

The term “immune-evading moiety,” as used herein, refers to a signaling peptide, or portion thereof, that reduces cellular phagocytosis through its interaction with a signal receptor protein expressed by phagocytic cells, such as macrophages and dendritic cells. In some embodiments, the immune-evading moiety blocks immune cell recognition or immune cell activation.

The term “targeting moiety,” as used herein, refers to an entity that guides a cell, such as for e.g., an enucleated cell, to a target tissue or cell. The targeting moiety can be virtually any biomolecule, including a protein, polypeptide, a sugar, a nucleic acid, or a small molecule, or portions thereof.

The term “transmembrane moiety,” as used herein, refers to an entity that spans (at least partially) the cell membrane of a cell (e.g., enucleated cell).

The terms “expression” or “expressing” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

As used herein, a “cell” generally refers to a biological cell.

As used herein, “enucleation” is the rendering of a cell to a non-replicative state, such as, for example, through removal of the nucleus.

As used herein, the term “cytoplast,” “cell without a nucleus,” or “enucleated cell” are used interchangeably to refer to a nucleus-free cell that was obtained from a previously nucleated cell (e.g., any cell described herein). In some embodiments, the nucleated cell comprises cell organelles and the cytoplast derived from the nucleated cell retains such organelles, which in some cases, enables cellular functions such as cell motility, protein synthesis, protein secretion, and the like. In some embodiments, “obtaining” does not involve differentiating the nucleated cell into an enucleated cell using natural processes or otherwise.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. The term “gene” is to be interpreted broadly, and may encompass mRNA, cDNA, cRNA and genomic DNA forms of a gene.

In some uses, the term “gene” encompasses the transcribed sequences, including 5′ and 3′ untranslated regions (5′-UTR and 3′-UTR), exons and introns. In some genes, the transcribed region may contain “open reading frames” that encode polypeptides. In some uses of the term, a “gene” comprises only the coding sequences (e.g., an “open reading frame” or “coding region”) necessary for encoding a polypeptide. In some embodiments, genes do not encode a polypeptide, for example, ribosomal RNA genes (rRNA) and transfer RNA (tRNA) genes. In some embodiments, the term “gene” includes not only the transcribed sequences, but in addition, also includes non-transcribed regions including upstream and downstream regulatory regions, enhancers and promoters. The term “gene” may encompass mRNA, cDNA and genomic forms of a gene.

The term “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments.

As used herein, the term “package” refers to a suitable solid matrix or material such as glass, plastic, paper, foil, and the like, capable of holding the individual kit components. For example, a package may be a glass vial or prefilled syringes used to contain suitable quantities of the pharmaceutical. The packaging material has an external label which indicates the contents and/or purpose of the kit and its components.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure, and may perform any function, known or unknown. A polynucleotide comprises one or more analogs (e.g., altered backbone, sugar, or nucleobase). Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

As used herein, the terms “polypeptide,” “peptide” and “protein” may be used interchangeably herein in reference to a polymer of amino acid residues. A protein may refer to a full-length polypeptide as translated from a coding open reading frame, or as processed to its mature form, while a polypeptide or peptide may refer to a degradation fragment or a processing fragment of a protein that nonetheless uniquely or identifiably maps to a particular protein. A polypeptide may be a single linear polymer chain of amino acids bonded together by peptide bonds between the carboxyl and amino groups of adjacent amino acid residues. Polypeptides may be modified, for example, by the addition of carbohydrate, phosphorylation, etc.

As used herein, the terms “fragment,” or “portion,” or equivalent terms may refer to a portion of an entity that has less than the full length of the entity and optionally maintains the function of the entity. In some embodiments, the entity is a protein.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions used in the relevant field) to hybridization with non-target sequences during a hybridization reaction. Typically, hybridizable sequences share a degree of sequence complementarity over all or a portion of their respective lengths, such as between 25%-100% complementarity, including greater than or equal to about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, may be measured by any suitable alignment algorithm, including but not limited to the Needleman-Wunsch algorithm (see e.g., the EMBOSS Needle aligner available at www.ebi.ac.uk/Tools/psa/emboss_needle/nucleotide.html, optionally with default settings), the BLAST algorithm. Optimal alignment may be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (e.g., gaps may be introduced in one or both of the candidate and reference sequences for optimal alignment and non-homologous sequences may be disregarded for comparison purposes). Alignment, for purposes of determining percent identity, may be achieved in various ways that are known in the relevant field. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” may be used to describe an event that takes place in an organism, such as a subject's body.

As used herein, the term “ex vivo” may be used to describe an event that takes place outside of an organism, such as subject's body. An “ex vivo” assay cannot be performed on a subject. Rather, it may be performed upon a sample separate from a subject. Ex vivo may be used to describe an event occurring in an intact cell outside a subject's body.

As used herein, the term “in vitro” may be used to describe an event that takes places contained in a container for holding laboratory reagent such that it is separated from the living biological source organism from which the material is obtained. In vitro assays may encompass cell-based assays in which cells alive or dead are employed. In vitro assays may also encompass a cell-free assay in which no intact cells are employed.

“Treat, “treating,” or “treatment,” as used herein, refers to alleviating or abrogating a disorder, disease, or condition; or one or more of the symptoms associated with the disorder, disease, or condition; or alleviating or eradicating a cause of the disorder, disease, or condition itself. Desirable effects of treatment may include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “effective amount” and “therapeutically effective amount,” as used interchangeably herein, generally refer to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component may be “pharmaceutically acceptable” in the sense of being compatible with the other ingredients of a pharmaceutical formulation. It may also be suitable for use in contact with the tissue or organ of humans and animals without excessive toxicity, irritation, allergic response, immunogenicity, or other problems or complications, commensurate with a reasonable benefit/risk ratio.

As used herein, the term “administration,” “administering” and variants thereof means introducing a composition or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and administration by another. A suitable route of administration allows the composition or the agent to perform its intended function. For example, if a suitable route is intravenous, the composition is administered by introducing the composition or agent into a vein of the subject. Administration may be carried out by any suitable route. In some embodiments, the administering is intravenous administration. In some embodiments, the administering is pulmonary administration. In some embodiments, the administering is inhalation.

The term “pharmaceutical composition” refers to a mixture of a composition disclosed herein with other chemical components, such as diluents or carriers (e.g., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition may facilitate administration of the composition to an organism. Multiple techniques of administering a compound exist in the art including, but not limited to, oral, injection, aerosol, parenteral, and topical administration.

The term “fusogenic protein”, as used herein, refers to a polypeptide that, when expressed on the surface of cell, such as the enucleated cell, facilitates fusion of cell-to-cell membranes of the cell expressing the fusogenic protein and a target cell.

While preferred embodiments of the present inventive concepts have been shown and described herein, it will be obvious to those skilled in the art that such embodiments are provided by way of example only. It is not intended that the inventive concepts be limited by the specific examples provided within the specification. While the inventive concepts have been described with reference to the aforementioned specification, the descriptions and illustrations of the embodiments herein are not meant to be construed in a limiting sense. Numerous variations, changes, and substitutions will now occur to those skilled in the art without departing from the inventive concepts. Furthermore, it shall be understood that all aspects of the inventive concepts are not limited to the specific depictions, configurations or relative proportions set forth herein which depend upon a variety of conditions and variables. It should be understood that various alternatives to the embodiments of the inventive concepts described herein may be employed in practicing the inventive concepts. It is therefore contemplated that the inventive concepts shall also cover any such alternatives, modifications, variations or equivalents. It is intended that the following claims define the scope of the inventive concepts and that methods and structures within the scope of these claims and their equivalents be covered thereby.

### Embodiments

Embodiment 1. A method for cell processing, the method comprising:
a) providing a composition comprising nucleated cells; and
b) enucleating a portion of the nucleated cells to produce an enucleated cell fraction using continuous flow centrifugation, wherein the portion of the nucleated cells comprises greater than or equal to about 70% of the nucleated cells.

Embodiment 2. The method of Embodiment 1, wherein the composition provided herein a) has a volume comprising between more than or equal to about 10 milliliters (mL) to about 10000 mL.

Embodiment 3. The method of Embodiment 2, wherein the composition has a volume comprising more than or equal to about 10 mL, about 20 mL, about 30 mL, about 40 mL, about 50 mL, about 60 mL, about 80 mL, about 100 mL, about 200 mL, about 300 mL, about 500 mL, about 1000 mL, about 2000 mL, about 3000 mL, about 4000 mL, about 5000 mL, about 6000 mL, about 7000 mL, about 8000 mL, about 9000 mL, or about 10000 mL.

Embodiment 4. The method of Embodiment 1, wherein the continuous flow centrifugation generates a density gradient that separates the enucleated cell fraction from the nucleated cells in the composition.

Embodiment 5. The method of Embodiment 4, wherein the density gradient comprises a polysaccharide density gradient.

Embodiment 6. The method of Embodiment 5, wherein the polysaccharide density gradient comprises a Ficoll density gradient.

Embodiment 7. The method of Embodiment 6, wherein the Ficoll density gradient comprises at least two, at least three, at least four, at least five, at least six, or at least seven ranges of the density gradient.

Embodiment 8. The method of Embodiment 7, wherein the Ficoll density gradient comprises about 25% Ficoll, about 17% Ficoll, about 16% Ficoll, about 15% Ficoll, or about 12.5% Ficoll.

Embodiment 9. The method of any one of Embodiments 1-8, wherein the portion of the nucleated cells comprises greater than or equal to about 75% of the nucleated cells.

Embodiment 10. The method of any one of Embodiments 1-9, wherein the portion of the nucleated cells comprises greater than or equal to about 80% of enucleated cells.

Embodiment 11. The method of any one of Embodiments 1-10, wherein the portion of the nucleated cells comprises greater than or equal to about 90% of enucleated cells.

Embodiment 12. The method of any one of Embodiments 1-11, wherein the enucleated cell fraction produced by performing the continuous flow centrifugation once comprises more than or equal to about: (i) 6×10⁷ of enucleated cells, (ii) 7×10⁷ of enucleated cells, (iii) 8×10⁷ of enucleated cells, (iv) 9×10⁷ of enucleated cells, (v) 10×10⁷ of enucleated cells, (vi) 15×10⁷ of enucleated cells, (vii) 20×10⁷ of enucleated cells, (viii) 50×10⁷ of enucleated cells, (ix) 100×10⁷ of enucleated cells, (x) 150×10⁷ of enucleated cells, (xi) 200×10⁷ of enucleated cells, or (xii) 250×10⁷ of enucleated cells.

Embodiment 13. The method of Embodiment 4, further comprising generating the density gradient comprising centrifuging a polysaccharide with acceleration spanning over at least about 10 minutes, at least about 20 minutes, at least about 30 minutes, at least about 40 minutes, or at least about 50 minutes.

Embodiment 14. The method of Embodiment 4, further comprising generating the density gradient comprising centrifuging a polysaccharide with acceleration spanning over about 10 minutes, about 20 minutes, about 30 minutes, about 40 minutes, or about 50 minutes.

Embodiment 15. The method of Embodiment 4, further comprising generating the density gradient comprising centrifuging a polysaccharide with acceleration spanning about 30 minutes.

Embodiment 16. The method Embodiment 4, wherein the enucleating in b) further comprises generating the density gradient comprising centrifuging a polysaccharide with minimal deceleration.

Embodiment 17. The method of Embodiment 6, wherein the enucleating in b) further comprises generating the density gradient comprising centrifuging a polysaccharide at a maximum centrifugal force of between about 30000 relative centrifugal force (RCF) to about 200000 RCF.

Embodiment 18. The method of Embodiment 17, wherein the enucleating in b) further comprises generating the density gradient comprising centrifuging a polysaccharide at a maximum centrifugal force of between about 50000 RCF to about 120000 RCF.

Embodiment 19. The method of any one of any one of Embodiments 1-18, wherein enucleating the portion of the nucleated cells to produce the enucleated cell fraction using the continuous flow centrifugation in b) is performed using an ultracentrifuge.

Embodiment 20. The method of any one of any one of Embodiments 1-19, wherein enucleating the portion of the nucleated cells to produce the enucleated cell fraction using the continuous flow centrifugation in b) is performed using fixed angle centrifugation or swinging bucket centrifugation.

Embodiment 21. The method of any one of Embodiments 1-20, wherein the nucleated cells comprise a heterologous polynucleotide.

Embodiment 22. The method of Embodiment 21, wherein the method further comprises inducing cell death of the nucleated cells that are not enucleated after b), wherein the cell death is induced by expressing at least one heterologous gene encoded by the heterologous polynucleotide.

Embodiment 23. The method of any one of previous Embodiments, wherein the continuous flow centrifugation generates zonal centrifugation for separating at least one enucleated cell from the nucleated cells.

Embodiment 24. The method of Embodiment 23, wherein the zonal centrifugation separates the at least one enucleated cell from the nucleated cells based on size of the at least one enucleated cell.

Embodiment 25. The method of Embodiments 23, wherein the zonal centrifugation separates the at least one enucleated cell from the nucleated cells based on mass of the at least one enucleated cell.

Embodiment 26. The method of Embodiment 23, wherein the zonal centrifugation separates the at least one enucleated cell from the nucleated cells based on size and mass of the at least one enucleated cell.

Embodiment 27. The method of Embodiment 4, wherein at least one density fraction is obtained from the density gradient, wherein the at least one density fraction comprises a mixed population of a subset of the nucleated cells and enucleated cells of the enucleated cell fraction.

Embodiment 28. The method of Embodiment 27, wherein the mixed population comprises at least 70% of the enucleated cells.

Embodiment 29. The method of Embodiment 27, wherein the mixed population comprises at least 99% of the enucleated cells.

Embodiment 30. A method for cell processing, the method comprising:
a) providing a composition comprising (i) a first subset of nucleated cells, and (ii) enucleated cells derived from a second subset of the nucleated cells, wherein the first subset of the nucleated cells comprises a heterologous polynucleotide encoding a heterologous gene product; and
b) expressing the heterologous gene product thereby inducing cell death of at least one nucleated cell of the first subset of the nucleated cells.

Embodiment 31. The method of Embodiment 30, wherein the heterologous polynucleotide comprises a promoter.

Embodiment 32. The method of Embodiment 31, wherein the promoter comprises an inducible promoter.

Embodiment 33. The method of Embodiment 32, wherein the inducible promoter is induced by contacting the nucleated cells with a temperature that is below 37° C.

Embodiment 34. The method of Embodiment 33, wherein the inducible promoter comprises dsrA or CIRP.

Embodiment 35. The method of Embodiment 32, wherein the inducible promoter is induced by contacting the nucleated cells with a temperature that is above 37° C.

Embodiment 36. The method of Embodiment 35, wherein the inducible promoter comprises HSP70, HSP90, GADD153, MDR1, or HSE-CMV.

Embodiment 37. The method of Embodiment 32, wherein the inducible promoter is induced by contacting the nucleated cells with a molecule.

Embodiment 38. The method of Embodiment 37, wherein the molecule comprises rtTA, TRE, TetR, Cumate, Rapamycin, Abscisic acid, IPTG, or Methallothionein.

Embodiment 39. The method of Embodiment 32, wherein the inducible promoter is induced by contacting the nucleated cells with light.

Embodiment 40. The method of Embodiment 39, wherein the inducible promoter comprises CIB1-CRY2 or GAL4-VVD.

Embodiment 41. The method of Embodiment 32, wherein the inducible promoter is induced by contacting with the nucleated cells with a hormone.

Embodiment 42. The method of Embodiment 41, wherein the inducible promoter comprises Estradiol-Gal4.

Embodiment 43. The method of Embodiment 31, wherein the promoter comprises a constitutively active promoter.

Embodiment 44. The method of Embodiment 43, wherein the constitutively active promoter is configured to activate transcription of the heterologous polynucleotide under conditions sufficient to express the heterologous gene product.

Embodiment 45. The method of Embodiment 44, wherein the heterologous gene product comprises herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase.

Embodiment 46. The method of Embodiment 44, wherein the heterologous gene product comprises FKBP or a caspase.

Embodiment 47. The method of Embodiment 44, wherein the heterologous gene product comprises an antigen.

Embodiment 48. The method of any one of Embodiments 30-47, wherein the heterologous polynucleotide is integrated into chromosome of the nucleated cells.

Embodiment 49. The method of any one of Embodiments 30-48, wherein the heterologous polynucleotide comprises a vector.

Embodiment 50. The method of any one of preceding Embodiments, further comprising cryopreserving the enucleated cell fraction to produce a cryopreserved enucleated cell fraction.

Embodiment 51. The method of any one of Embodiments 1-50, further comprising thawing the cryopreserved enucleated cell fraction, wherein, following the thawing, an enucleated cell of the cryopreserved enucleated cell fraction is as viable as an otherwise comparable enucleated cell that was not cryopreserved.

Embodiment 52. The method of Embodiment 1 or Embodiment 30, wherein the nucleated cells comprise stem cells.

Embodiment 53. The method of Embodiment 52, wherein the stem cells comprise induced pluripotent stem cells (iPSCs), adult stem cells, mesenchymal stromal cells, embryonic stem cells, fibroblasts, or immortalized cells from a cell line, or a combination thereof.

Embodiment 54. The method of Embodiment 53, wherein the nucleated cells comprise the mesenchymal stromal cells.

Embodiment 55. The method of any one of Embodiments 1-54, wherein the enucleated cells lack a nucleus and comprise one or more intracellular organelles for synthesis or secretion of an exogenous polypeptide in absence of the nucleus.

Embodiment 56. The method of Embodiment 55, wherein the exogenous polypeptide comprises a therapeutic agent.

Embodiment 57. The method of any one of Embodiments 1-56, wherein the enucleated cells comprise at least one targeting moiety.

Embodiment 58. The method of any one of Embodiments 1-57, wherein the enucleated cells comprise at least one fusogenic moiety.

Embodiment 59. The method of any one of Embodiments 1-58, wherein the enucleated cells comprise at least one immune evasion moiety.

Embodiment 60. The method of any one of Embodiments 1-59, wherein the enucleated cells comprise at least one therapeutic moiety.

Embodiment 61. The method of any one of Embodiments 1-60, wherein the enucleated cell of the enucleated cell fraction has a diameter comprising less than or equal to about 10%, about 20%, about 30%, about 40%, about 50%, about 60%, about 70%, about 80%, about 90%, about 95%, or about 99% of an average diameter of the nucleated cells provided in a).

Embodiment 62. The method of any one of Embodiments 1-60, wherein the enucleated cell of the enucleated cell fraction has a diameter comprising less than or equal to about 70% of an average diameter of the nucleated cells provided in a).

Embodiment 63. The method of any one of Embodiments 1-62, wherein the enucleated cell of the enucleated cell fraction has a diameter comprising more than or equal to about 5 μm, about 10 μm, about 20 μm, about 30 μm, about 40 μm, about 50 μm, about 60 μm, about 70 μm, about 80 μm, or about 90 μm.

Embodiment 64. The method of any one of Embodiments 1-62, wherein the enucleated cell of the enucleated cell fraction comprises a diameter comprising between about 10 micrometer (μm) to about 100 μm.

Embodiment 65. The method of Embodiment 64, wherein the diameter comprises about 8 μm.

Embodiment 66. A composition comprising:
a) enucleated cells obtained from a first subset of a plurality of nucleated cells; and
b) a second subset of the plurality of nucleated cells, wherein a nucleated cell of the second subset of the plurality of the nucleated cells comprises a heterologous polynucleotide encoding a heterologous gene product configured to induce cell death of the nucleated cell.

Embodiment 67. The composition of Embodiment 66, wherein the heterologous polynucleotide comprises a promoter configured to activate transcription of the heterologous polynucleotide under conditions sufficient to express the heterologous gene product.

Embodiment 68. The composition of Embodiment 67, wherein the promoter comprises an inducible promoter configured to activate transcription of the heterologous polynucleotide under conditions sufficient to express the heterologous gene product when induced.

Embodiment 69. The composition of Embodiment 68, wherein the inducible promoter is induced by contacting the nucleated cells with a temperature that is below 37° C.

Embodiment 70. The composition of Embodiment 69, wherein the inducible promoter comprises dsrA or CIRP.

Embodiment 71. The composition of Embodiment 68, wherein the inducible promoter is induced by contacting the nucleated cell with a temperature that is above 37° C.

Embodiment 72. The composition of Embodiment 71, wherein the inducible promoter comprises HSP70, HSP90, GADD153, MDR1, or HSE-CMV.

Embodiment 73. The composition of Embodiment 68, wherein the inducible promoter is induced by contacting the nucleated cell with a molecule.

Embodiment 74. The composition of Embodiment 73, wherein the molecule comprises rtTA, TRE, TetR, Cumate, Rapamycin, Abscisic acid, IPTG, or Methallothionein.

Embodiment 75. The composition of Embodiment 68, wherein the inducible promoter is induced by contacting the nucleated cell with light.

Embodiment 76. The composition of Embodiment 75, wherein the inducible promoter comprises CIB1-CRY2 or GAL4-VVD.

Embodiment 77. The composition of Embodiment 68, wherein the inducible promoter is induced by contacting the nucleated cell with a hormone.

Embodiment 78. The composition of Embodiment 77, wherein the inducible promoter comprises Estradiol-Gal4.

Embodiment 79. The composition of Embodiment 67, wherein the promoter comprises a constitutively active promoter.

Embodiment 80. The composition of Embodiment 79, wherein the constitutively active promoter is configured to activate transcription of the heterologous polynucleotide under conditions sufficient to express the heterologous gene product.

Embodiment 81. The composition of Embodiment 80, wherein the heterologous gene product comprises herpes simplex virus-thymidine kinase (HSV-TK), cytosine deaminase (CD), Varicalla-zoster-TK (VZV-TK), Nitroreductase, Carboxypeptidase G2 (CPG2), Cytochrome P450, or purine nucleoside phosphorylase.

Embodiment 82. The composition of Embodiment 80, wherein the heterologous gene product comprises FKBP or a caspase.

Embodiment 83. The composition of Embodiment 80, wherein the heterologous gene product comprises an antigen.

Embodiment 84. The composition of any one of Embodiments 66-86, wherein the heterologous polynucleotide is integrated into chromosome of the nucleated cell.

Embodiment 85. The composition of any one of Embodiments 66-84, wherein the heterologous polynucleotide comprises a vector.

Embodiment 86. A composition comprising:
a) enucleated cells obtained from a first subset of a plurality of nucleated cells; and
b) a second subset of the plurality of nucleated cells, wherein less than or equal to about 0.10% by volume of the composition comprises the second subset of the plurality of the nucleated cells.

Embodiment 87. The composition of any one of Embodiments 66-86, wherein the plurality of nucleated cells comprises stem cells.

Embodiment 88. The composition of Embodiment 87, wherein the stem cells comprise induced pluripotent stem cells (iPSCs), adult stem cells, mesenchymal stromal cells, embryonic stem cells, fibroblasts, or immortalized cells from a cell line, or a combination thereof.

Embodiment 89. The composition of Embodiment 88, wherein the nucleated cells comprise the mesenchymal stromal cells.

Embodiment 90. The composition of any one of Embodiments 66-89, wherein the enucleated cells lack a nucleus and comprise one or more structural features of the plurality of nucleated cells.

Embodiment 91. The composition of Embodiment 90, wherein the one or more structural features comprise one or more intracellular organelles, one or more tunneling nanotubes, or a combination thereof.

Embodiment 92. The composition of any one of Embodiments 66-91, wherein the enucleated cells lack a nucleus and comprise one or more intracellular organelles for synthesis or secretion of an exogenous polypeptide in absence of the nucleus.

Embodiment 93. The composition of Embodiment 91 or Embodiment 92, wherein the one or more intracellular organelles comprise a Golgi apparatus, an endoplasmic reticulum, or a combination thereof.

Embodiment 94. The composition of Embodiment 92, wherein the exogenous polypeptide comprises a therapeutic agent.

Embodiment 95. The composition of any one of Embodiments 66-94, wherein the enucleated cells comprise at least one targeting moiety.

Embodiment 96. The composition of any one of Embodiments 66-95, wherein the enucleated cells comprise at least one fusogenic moiety.

Embodiment 97. The composition of any one of Embodiments 66-96, wherein the enucleated cells comprise at least one immune evasion moiety.

Embodiment 98. The composition of any one of Embodiments 66-97, wherein the enucleated cells comprise at least one therapeutic moiety.

Embodiment 99. The composition of any one of Embodiments 66-98, wherein the enucleated cell of an enucleated cell fraction has a diameter comprising less than or equal to at least about 10%, about 20%, about 30%, about 40%, about 50%, about 60%, about 70%, about 80%, about 90%, about 95%, or about 99% of an average diameter of the nucleated cells provided in a).

Embodiment 100. The composition of any one of Embodiments 66-99, wherein the enucleated cell of the enucleated cell fraction has a diameter comprising less than or equal to about 70% of an average diameter of the nucleated cells provided in a).

Embodiment 101. The composition of any one of Embodiments 66-100, wherein the enucleated cell of the enucleated cell fraction has a diameter comprising between about 10 μm to about 100 μm.

Embodiment 102. The composition of Embodiment 101, wherein the enucleated cell of the enucleated cell fraction has a diameter comprising more than or equal to about 1 μm, about 5 μm, about 8 μm, about 10 μm, about 20 μm, about 30 μm, about 40 μm, about 50 μm, about 60 μm, about 70 μm, about 80 μm, about 90 μm, or about 100 μm.

Embodiment 103. The composition of Embodiment 102, wherein the diameter comprises about 8 μm.

Embodiment 104. The composition of any one of Embodiments 66-103, wherein the composition is in a dosage form suitable for intravenous administration.

Embodiment 105. The composition of Embodiment 104, wherein the dosage form comprises a solid dosage form.

Embodiment 106. The composition of any one of Embodiments 66-105, wherein the composition comprises a tablet, a pill, a powder, a capsule, solid dispersion, solid solution, bioerodible dosage form, a controlled release formulation, a pulsatile release dosage form, a multiparticulate dosage form, a bead, a pellet, or a granule.

Embodiment 107. The composition of any one of Embodiments 66-106, wherein a total number of the enucleated cells in the composition comprises more than or equal to about 10 million enucleated cells, about 20 million enucleated cells, about 30 million enucleated cells, about 40 million enucleated cells, about 45 million enucleated cells, about 50 million enucleated cells, about 55 million enucleated cells, about 60 million enucleated cells, about 65 million enucleated cells, about 70 million enucleated cells, about 75 million enucleated cells, about 80 million enucleated cells, about 90 million enucleated cells, or about 100 million enucleated cells.

Embodiment 108. The composition of any one of Embodiments 66-107, wherein the enucleated cell is further cryopreserved to produce a cryopreserved enucleated cell.

Embodiment 109. The composition of Embodiment 108, wherein the cryopreserved enucleated cell fraction is thawed, wherein, following the thawing, the enucleated cell of the cryopreserved enucleated cell fraction is as viable as an otherwise comparable enucleated cell that was not cryopreserved.

Embodiment 110. The composition of any one of Embodiments 66-107, wherein the enucleated cell exhibits viability after cryohibernation.

Embodiment 111. The composition of Embodiment 110, wherein the enucleated cell exhibits the viability following the cryohibemation as measured at 24 hours following the cryohibernation that is equal to or greater than the viability of a comparable enucleated cell that is not cryohibernated.

Embodiment 112. The composition of any one of Embodiments 66-107, wherein the enucleated cell exhibits viability after cryopreservation.

Embodiment 113. The composition of Embodiment 112, wherein the enucleated cell exhibits the viability following the cryopreservation as measured at 24 hours following the cryopreservation that is equal to or greater than the viability of a comparable enucleated cell that is not cryopreserved.

Embodiment 114. The composition of any one of Embodiments 66-113, wherein the composition is purified.

Embodiment 115. The composition of any one of Embodiments 66-113, wherein the composition is lyophilized.

Embodiment 116. The composition of any one of Embodiments 66-115, wherein the enucleated cells and the plurality of the nucleated cells are at the same stage of cell differentiation.

Embodiment 117. The composition of any one of Embodiments 66-116, wherein the enucleated cells are not obtained from the plurality of the nucleated cells by cell differentiation.

Embodiment 118. The composition of any one of Embodiments 66-116, wherein the enucleated cells are not terminally differentiated cells.

Embodiment 119. The composition of any one of Embodiments 66-116, wherein the enucleated cells are not platelets.

Embodiment 120. The composition of any one of Embodiments 66-116, wherein the enucleated cells are not obtained from platelet lineage cells.

Embodiment 121. The composition of any one of Embodiments 66-116, wherein the enucleated cells are not red blood cells.

Embodiment 122. The composition of any one of Embodiments 66-116, wherein the enucleated cells are not obtained from red blood cell lineage cells.

Embodiment 123. A plurality of enucleated cells comprising a plurality of the enucleated cells of any one of Embodiments 66-122.

Embodiment 124. A pharmaceutical composition comprising:
a) the enucleated cells of any one of Embodiments 66-112; and
b) a pharmaceutically acceptable: excipient, carrier, or diluent.

Embodiment 125. The pharmaceutical composition of Embodiment 124, wherein the pharmaceutical composition is in a unit dose form.

Embodiment 126. The pharmaceutical composition of Embodiment 124 or 125, wherein the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof, to a subject.

Embodiment 127. The pharmaceutical composition of Embodiment 126, wherein the pharmaceutical composition is formulated for administering intravenously.

Embodiment 128. The pharmaceutical composition of any one of Embodiments 124-127, further comprising at least one additional active agent.

Embodiment 129. The pharmaceutical composition of Embodiment 128, wherein the at least one additional active agent comprises a cytokine, a growth factor, a hormone, an enzyme, a small molecule, a compound, or a combination thereof.

Embodiment 130. A kit comprising:
a) the composition of any one of Embodiments 66-123 or the pharmaceutical composition of Embodiments 124-129; and
b) a container.

### EXAMPLES

The following illustrative examples are representative of embodiments of the stimulation, systems, and methods described herein and are not meant to be limiting in any way.

### Example 1—Successful Enucleation and Survival of Mammalian Cells

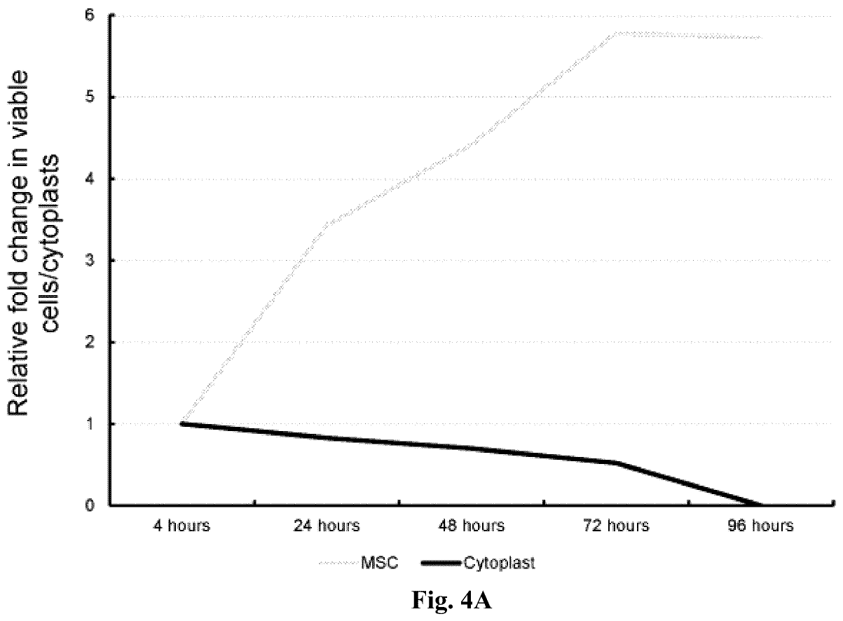

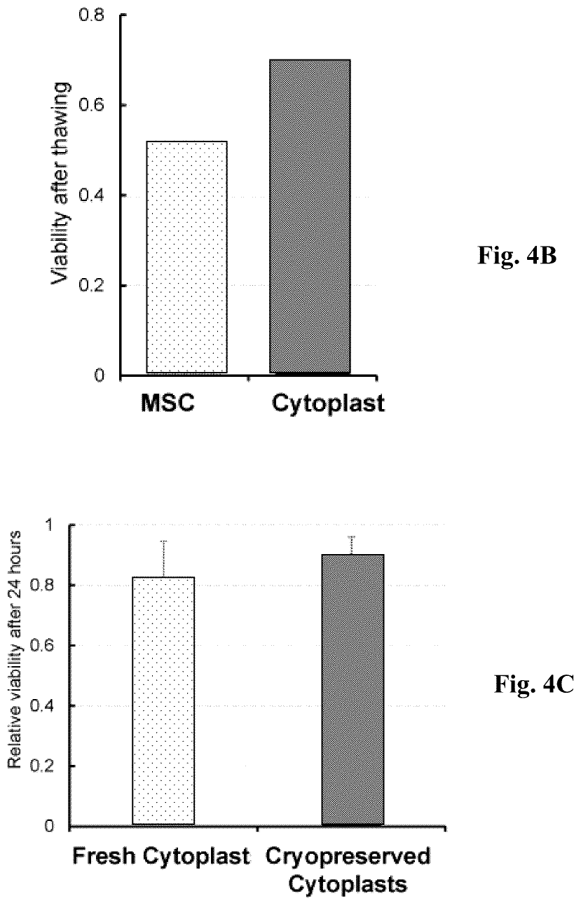

The enucleation efficiency and recovery rate of various types of mammalian cells (e.g., mesenchymal stem cells, neutrophils, fibroblast, and natural killer cells) was determined. After removal of the mammalian cells from the cell culture plates, the mammalian cells were enucleated by density gradient centrifugation using discontinuous Ficoll gradients, high-speed centrifugation (FIG. 4A-FIG. 4C). Table 1 summarizes the results of enucleation using a suspension protocol. Enucleation efficiency and cell viability was the highest in both hTERT transformed and primary mesenchymal stem cells (MSCs), as well as in fibroblasts and neutrophils. Table 2 summarizes the results of enucleation using an adherent protocol. Enucleation efficiency was greater than 70% in both mesenchymal stem cells and macrophages. This experiment showed that various types of mammalian cells could undergo enucleation using any of the methods described herein.

TABLE 1
Enucleation efficiency and viability
determinations of mammalian cells

| | | Enucle- | | Viabil- | |
|---|---|---|---|---|---|
| | | ation | | ity | |
| | | Effi- | Recovery | after 24 | Yield per |
| Cell type | | ciency | Rate | hours | run |
| MSC cells | AD-MSC | 90%-95% | 60%-90% | 80%-95% | 12-15M |
| | (hTERT) | | | | |
| | UC-MSC | 85%-90% | 60%-80% | 80%-95% | 10-15M |
| | (primary) | | | | |
| | BM-MSC | 80%-90% | 40%-50% | 80%-90% | ~8M |
| | (primary) | | | | |
| NK cells | NKL | 50%-85% | 20%-50% | 50%-75% | ~8M |
| | NK-92 | 70%-90% | 20%-40% | 20%-40% | ~5M |
| Macro- | RAW | 85%-95% | 40%-70% | 20%-40% | ~15M |
| phages | 264.7 | | | | |
| Neutro- | HL-60 | 60%-98% | 20%-40% | 60%-80% | ~15M |
| phils | | | | | |
| Fibro- | L929 | 70%-90% | 50%-70% | 70%-90% | ~15M |
| blasts | NIH3T3 | 70%-80% | 40%-50% | 70%-80% | ~9M |

Enucleation efficiency = enucleated cells versus total recovered cells;
Recovery rate = recovered cells versus total input cells used for enucleation.
Viability after 24 hours = live cells measured by Trypan blue staining versus total cells;
Yield per run = the number of cytoplasts harvested for each run;
M = million cells
AD-MSC (hTERT) = human hTERT immortalized adipose-derived mesenchymal stem cells;
BM-MSC (primary) = human primary bone marrow-derived mesenchymal stem cells;
NK = natural killer cells.

TABLE 2
Enucleation efficiencies and viability
determinations of mammalian cells

| | | Enucle- | | Viabil- | |
|---|---|---|---|---|---|
| | | ation | | ity | |
| | | Effi- | Recovery | after 24 | Yield |
| Cell type | | ciency | Rate | hours | per run |
| MSC cells | AD-MSC | 70%-95% | 40%-60% | 80%-95% | 1M |
| | (hTERT) | | | | |
| Macrophages | RAW 264.7 | 85%-95% | 40%-70% | 10%-30% | ~1M |

Enucleation efficiency = enucleated cells versus total recovered cells;
Recovery rate = recovered cells versus total input cells used for enucleation.
Viability after 24 hours = live cells measured by Trypan blue staining versus total cells;
Yield per run = the number of cytoplasts harvested for each run;
M = million cells

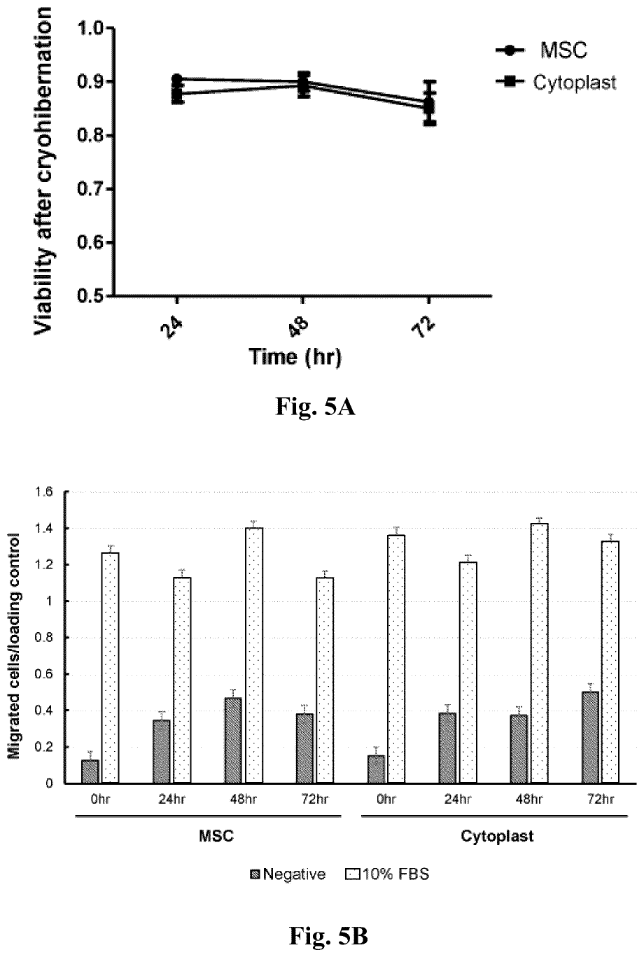

Next, the survival of cytoplasts was determined across 96 hours (FIG. 4A). Whereas MSC proliferated over-time, cytoplasts did not. Instead, the relative fold change in viable cytoplasts remained fairly constant for 72 hours before declining at 96 hours. Thus, cytoplast survival spanned 3-4 days. As most cell-based therapies are not used immediately, the viability of cytoplasts after cryopreservation was determined. Surprisingly, the viability of cytoplast after cryopreservation was greater than the viability of MSC following cryopreservation (FIG. 4B). Cytoplasts plated immediately after enucleation and cytoplasts recovered from cryopreservation displayed similar relative cell viability after 24 hours (FIG. 4C). This experiment showed that cytoplasts survival was not affected by cryopreservation. Additionally, the viability of cytoplasts after cryohibernation was similar to the viability of MSC following cryohibernation (FIG. 5A). Cytoplasts recovered after cryohibemation for various lengths of time were able to undergo induced migration in a Boyden chamber assay similar to MSCs recovered after cryohibernation, (FIG. 5B).

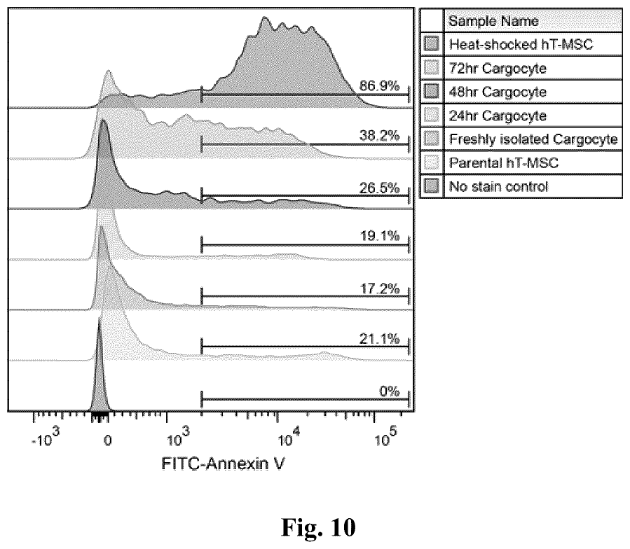

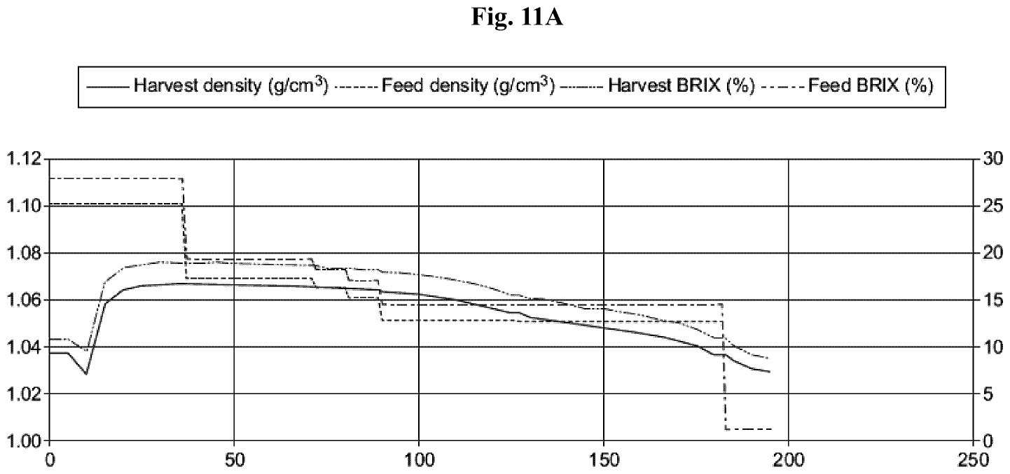

Additional viability study of the cytoplast generated by the methods described herein. FIG. 10 illustrates a cell surface staining of FITC-labeled Annexin V on MSCs or cytoplasts analyzed by flow cytometry. Data were analyzed in Flowjo and normalized to mode. Parental MSC=Non-engineered MSC; Isotype control=MSC stained with isotype matching IgG. 2 hr (hour)/24 hr/48 hr/72 hr Cytoplast=MSC-derived cytoplast analyzed at indicated time-point post-enucleation; Heat-shocked cells served as a positive control for apoptotic MSC cell death. Representative results from 3 independent experiments shown. After 3 Days post enucleation cytoplasts exhibited apoptosis as indicated by Annexin V Staining and FACS.

Next, a large-scale production of cells was set up ex vivo, followed by large-capacity density gradient centrifugation and enucleation, which lead to the generation of a therapeutic cytoplast. In one embodiment, the therapeutic cytoplast is loaded with therapeutic cargo (e.g., mRNA, drugs, peptides, etc.) for disease treatment. In another embodiment, the therapeutic cytoplast is prepared for immediate use (e.g., for intravenous injection (IV), intraperitoneal injection (IP), tissue, or in vitro applications) for diagnostic use.

### Example 2. Enucleated Cells Retain Intact and Functional Organelles

After determining whether cytoplasts could retain viability after cryopreservation, flow cytometry analysis were performed in order to determine whether the cell surface marker profile of MSC-derived cytoplasts differed from bone-marrow derived MSC. Both MSC-derived cytoplasts and bone-marrow derived MSCs maintained cell surface expression of CD45, CD90, CD44, CD146, and CD166. The cytoplasts reorganized the cytoskeleton, spread on matrix proteins in 2D and 3D culture systems, and formed tunneling nanotubes, which can transfer bioproducts between cells of the same or different origin. Organelle-staining indicated that Golgi, ER, F-actin cytoskeleton, lysosomes, endosomes, microtubules, and mitochondria remain intact in cytoplasts. Furthermore, cytoplasts exhibited homing potential in vitro. Cytoplasts readily migrated on extracellular matrix proteins and migrated directionally towards soluble chemokine gradients (e.g., via chemosensing). Notably, cytoplasts transfected exogenously with purified mRNAs produced functional intracellular proteins, which could mimic therapeutic mRNA applications being developed for a variety of clinical uses and disease-states. This also demonstrates that the machineries for mRNA translation and protein synthesis operate normally in cytoplasts in the absence of a nucleus, and thus can be used to produce bioactive molecules with therapeutic value.

Cytoplasts transfected exogenously with purified mRNA encoding known secreted proteins produce functional extracellular proteins in conditioned culture media, indicating that the ER/Golgi and secretory pathways operate normally in cytoplasts in the absence of a nucleus. In addition, treatment of macrophages and endothelial cells with cytoplast-conditioned media containing secreted proteins activated key signal transduction responses in these cells. These results show proof of concept that cytoplasts could be used as novel vehicles to produce and deliver secreted proteins and biomolecules with therapeutic value. Cytoplasts can be loaded with various cargo including, but not limited to, siRNA, shRNA, mRNA, DNA plasmids, peptides, and chemotherapeutic agents.

### Example 3. Enucleated Cells Can Express Functional Cell Surface Proteins

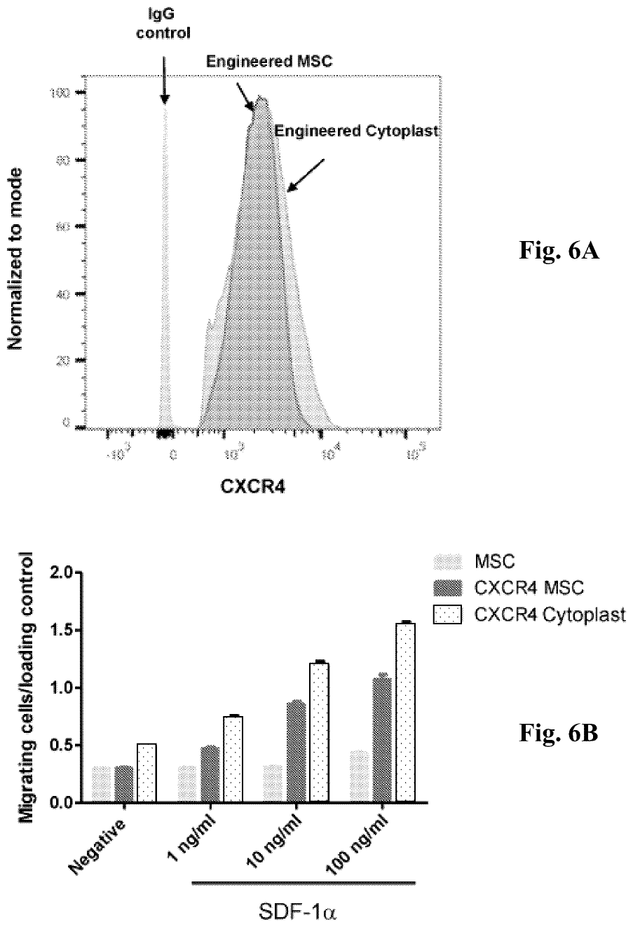

As shown in FIG. 6A, the engineered MSCs expressing CXCR4 and engineered MSC-derived cytoplasts expressing CXCR4 express comparable levels of CXCR4, as determined by flow cytometry. To determine whether engineered cytoplasts can express functional cell surface proteins, MSCs and MSC-derived cytoplasts expressing CXCR4 receptors were allowed to migrate towards various concentrations of SDF-la. As shown in FIG. 6B, MSC-derived cytoplasts engineered to express functional CXCR4 can migrate towards SDF-la, and cell migration increases with increasing concentrations of SDF-la. Furthermore, the number of migrating MSC-derived cytoplasts was greater than the number of migrating MSCs expressing CXCR4 (FIG. 6A and FIG. 6B).

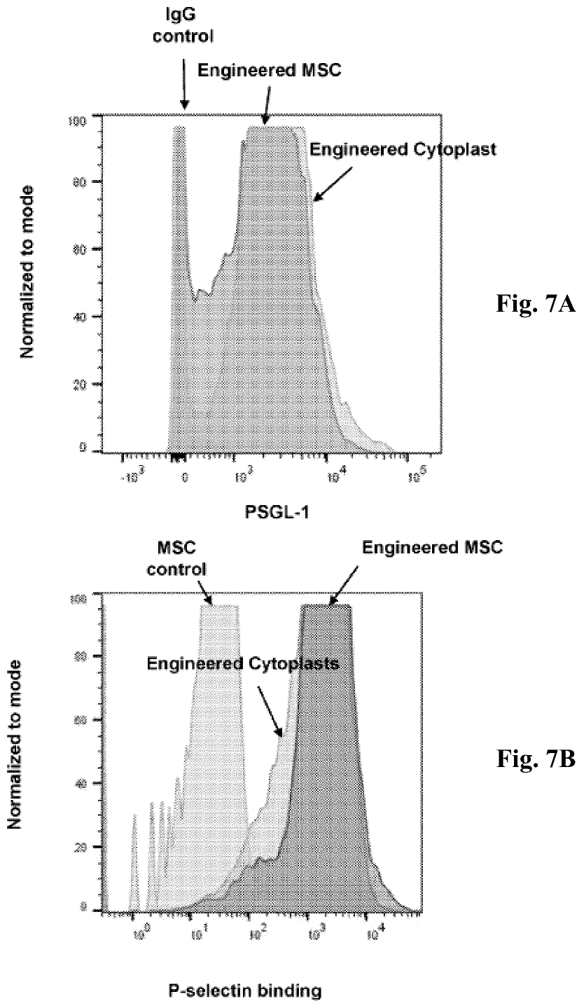

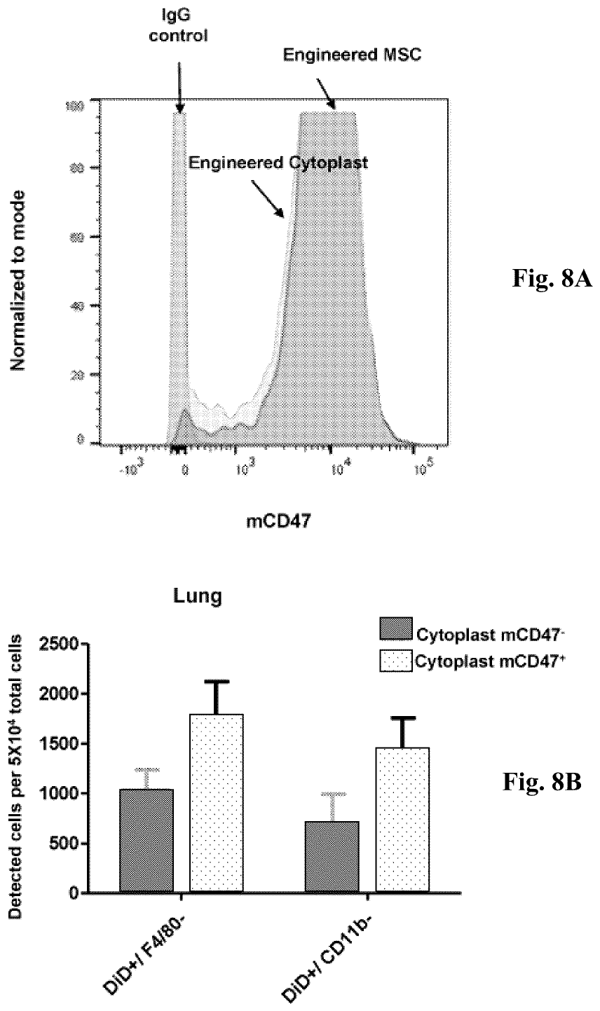

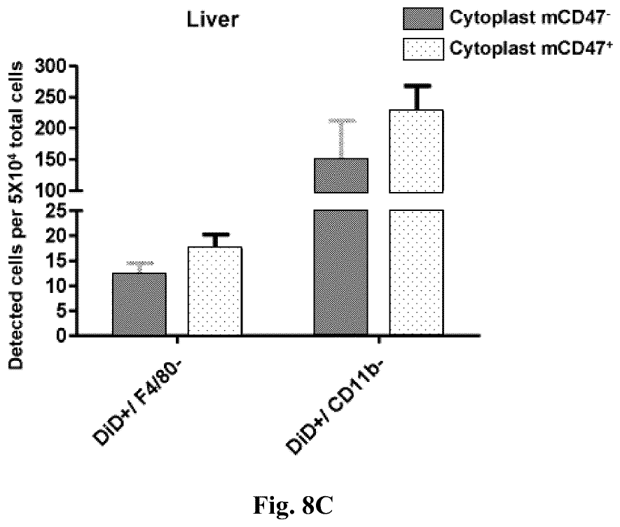

FIG. 7A and FIG. 7B show that MSC-derived cytoplasts can be engineered to express functional cell adhesion proteins known to mediate cell adhesion to the inflamed vasculature. FIG. 8A-FIG. 8C show that MSC-derived cytoplasts can be engineered to express cell proteins known to modulate macrophage interactions and phagocytosis of therapeutic cells.

### Example 4. 3D-Cultured Enucleated Cells Show Better Biodistribution In Vivo

MSCs were cultured in 3D-hanging drops (3D MSCs) then enucleated to generate 3D cytoplasts. Healthy MSCs were harvested from 2D-cultured plates by Trypsin and resuspended in fresh α-MEM (ThermoFisher 12561056) full medium (16.5% Premium FBS, 1% Antibiotic-Antimycotic, 1% Glutamax, 1% HEPES) at 1.43 million cells/ml. The lid of a 15 cm plate was opened completely and 20 ml PBS was added to the plate. A multichannel pipette was used to make droplets on the lid of the plate at 35 μl per droplet (approx. 50,000 cells/droplet). About 100-120 droplets were placed on each lid. The lid was closed, and the plate was placed back into the incubator. Droplets were cultured for 2 days, then harvested by cell lifter and collected into 15 ml tubes (approx. 300 droplets per tube). The tubes were centrifuged for 5 minutes at 1,200 rpm. The supernatant was removed, and the tubes were washed twice with PBS. All P BS was then removed and 7.5 ml of freshly thawed 0.25% Trypsin-EDTA (ThermoFisher 25200114) was added to each tube. The tubes were incubated in a water bath for 4 minutes. The droplets were gently pipetted with 1 ml pipettes with low-retention tips about 10-20 times and incubated in the water bath for another 4 minutes. The droplets were again gently pipetted with 1 ml pipettes with low-retention tips about 10-20 times until most of the droplets were dissociated. 7.5 ml of full serum medium (GlutaMAX Supplement (Gibco 35050061); Fetal Bovine Serum—Premium Select (Atlanta Biologicals S11550); HEPES (1 M) (Gibco 15630080); antibiotic-Antimycotic (100×) (Gibco 15240062)) was added to each tube and the tubes were centrifuged for 10 minutes at 1,200 rpm. The dissociated cells were washed with 10 ml of full serum medium, and the cells were resuspended with 5 ml full serum medium. The cells were passed through a 70 μm cell filter and then the filter was washed with 5 ml full serum medium. The cells were counted and resuspended with pre-treated 12.5% Ficoll at more than 10M/ml. 30-40M cells were used for each enucleation tube. Subsequently, the protocol for enucleation described above was followed.

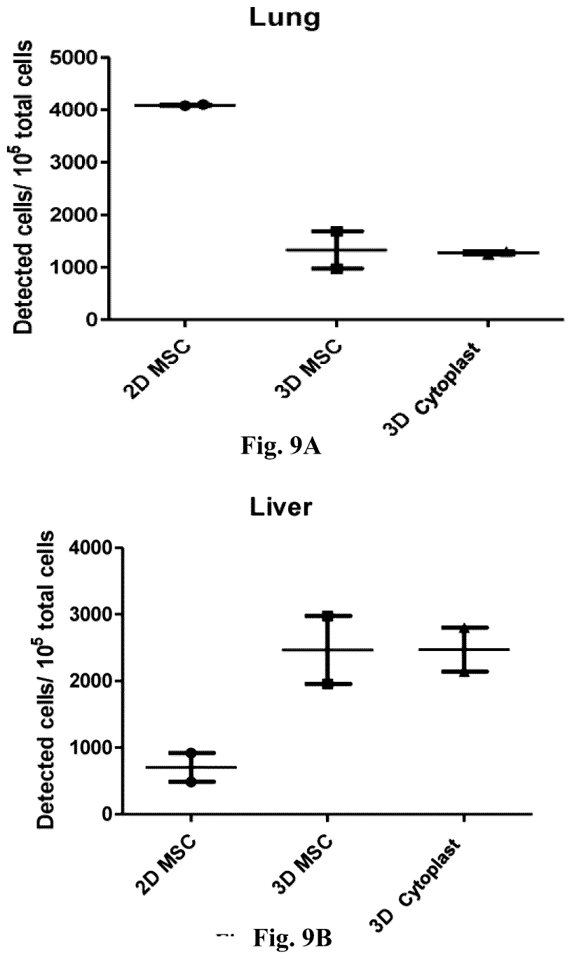

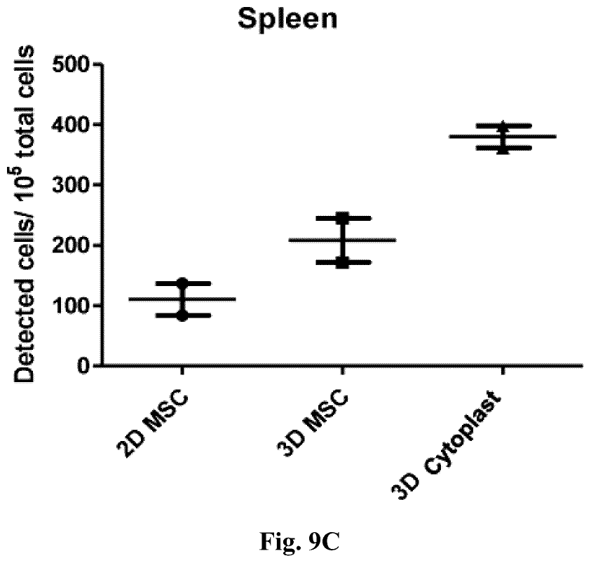

DiD labeled normal 2D-cultured MSCs (2D MSC), 3D MSCs or 3D cytoplasts were retro-orbitally injected into Ba1B/C mice respectively. Indicated tissues were harvested 24 hours after injection and DiD labeled cells analyzed by FACS. FIG. 9A-FIG. 9C show the successful generation of 3D-derived cytoplasts from 3D-cultured MSCs and also shows the 3D-derived cytoplasts have less lung trapping and better biodistribution to peripheral organs than 2D-cultured cells after injection into the circulation. This is expected to greatly improve their therapeutic ability to locate and deliver cargo to tissues.

### Example 5. Generating the Enucleated Cells

### Enucleation of Mesenchymal Stromal Cells (MSC)

Preparation of 50% Ficoll solution: In a glass beaker shielded from light, grams of Ficoll (PM400, GE Healthcare 17-0300-500) were dissolved in an equivalent number of milliliters ultrapure water (Invitrogen 10977-015) by continual magnetic stirring for 24 hours at room temperature. The mixture was then autoclaved for 30 minutes. Once the mixture was cooled, it was stirred again to ensure uniform consistency. The refractive index was measured on a refractometer (Reichert 13940000) and was in the range of 1.4230-1.4290. Aliquots were stored at −20 degrees Celsius.

Preparation of 2X MEM: For each 50 ml quantity, 10 milliliters (mL) 10X MEM (Gibco, 11430-030), 2.94 mL exactly Sodium Bicarbonate (7.5%, Gibco, 25080-094), 1 mL 100X Pen-Strep (Gibco 15140-122) and 36 mL ultrapure water (Invitrogen 10977-015) was used. The solution was then filtered through 0.22 um membrane flask (Olympus 25-227) and stored at 4 degrees Celsius.

On the day before enucleation, MSCs were seeded at 2.5 M per 15 cm plate (Olympus 25-203) in 20 mL MSC medium DMEM 1X (Gibco 12561-056); 16.5% premium FBS (Atlanta Biologics S1150); 1% HEPES 1M (Gibco 15630-80); 1% Anti-Anti 100X (Gibco 15240-062); 1% Glutamax 100X (Gibco 35050-061)]. Next, Cytochalasin B (Sigma Aldrich C6762) was added to the 2X MEM (2 μM/mL final concentration).

Preparation of Ficoll gradients: 2× CytoB was added to 50% Ficoll aliquots at 1:1 dilution to make 25% Ficoll stock concentration. Next, 17%, 16%, 15% and 12.5% Ficoll were made by diluting 25% Ficoll with the appropriate volume of 1X MEM buffer (2X MEM containing Cytochalasin B added to ultrapure water at 1:1 dilution). The dilutions were equilibrated in a CO₂ incubator for at least 1 hour covered with loose cap. The Ficoll gradients were then poured into 13.2 mL ultra-clear tubes (Beckman, 344059), and incubated overnight (6-18 hours) in the CO₂ incubator.

On the day of enucleation, 12-25M MSC (ideally 20M) were collected into each tube for enucleation. Media was aspirated, and the cells washed once with phosphate buffered saline (PBS) (GIBCO 14190-144). Five mL of TrypLE-Select (Gibco, 12563011) was added to each plate, and incubated up to 5 minutes. When 90% of the cells were detached, 5 mL full MSC media was added, and the cells were collected into 50 ml tubes (3-4 plates/tube). The tubes were then centrifuged at 1, 200 rpm for 5 minutes. The pellet was resuspended in 10 mL PBS. Cells were counted, pelleted, and re-suspended with 12.5% Ficoll. Next, the cell-Ficoll mixture was dropwise passed through a 40 μm cell strainer (Falcon 352340) into a new 50 mL tube. Using a syringe, 3.2 mL of cell suspension was slowly loaded onto the pre-made gradients. One mL of 1X MEM buffer was added at the final (top) layer with syringe. The tubes were then loaded into rotor buckets, balanced, and run in the ultracentrifuge (Beckman, L8M) for 60 minutes, 26,000 rpm, 31° C., Accel 7, Deccel 7. At the end of the centrifugation, there were three layers: one near the top of the 12.5% (cytoplasts and debris), one near the 12.5/15% interface (cytoplasts), and a pellet at the bottom of the 25% (karyoplasts). The layers above 15% Ficoll solution were collected into 15 ml conical tubes. The collected layers are then diluted with more than 4 volumes warm serum-free MSC medium (i.e. 3 mL of Ficoll and filled with up to 15 mL media). After gently mixing, the mixture was pelleted for 10 minutes at 1,200 rpm. Following three washes with warm serum-free MSC medium, the cells were resuspended in media according to the experimental protocol, e.g., transfection media vs. migration media vs. serum free media vs. full media. Efficiency of enucleation was determined in a 12-well plate by adding full MSC media with 1:2000 dilution Vybrant® Dyecycle™ Green (Molecular Probes V35004) or 1:5000 dilution Hoechst 33342. A small volume of each layer was added to each well and allowed to attach/stain for 10 minutes in the incubator. The percentage of negative cytoplasts per population was determined by epifluorescent microscopy.

Cytoplast mRNA Transfection

1 M cytoplasts were suspended with warm 1 ml amino acid-free α-MEM full medium (ThermoFisher 12561056; 16.5% Premium fetal bovine serum (FBS), 1% Glutamax (Gibco 35050061), 1% HEPES (Gibco 15630080)). 1 μg mRNA was diluted with warm opti-MEM and mixed with pipet at least 20 times. 4 μl lipofectamine-3000 (ThermoFisher L300015) was added to 46 μl warm opti-MEM (ThermoFisher 31985062) and mixed with pipet for at least 20 times. The ratio of mRNA and lipofectamine-3000 was 1:4 (w/v). The mRNA and lipofectamine-3000 dilutions were mixed with pipet for at least 20 times and incubated at room temperature for 15 minutes. The mRNA and lipofectamine-3000 mixture was added to the cytoplast suspension, mixed well and incubated at 37° C. for 30 minutes. The suspension was shaken every 5 minutes to prevent cell clumping. After incubation, the cells were centrifuged, and re-suspended in normal α-MEM full medium (16.5% Premium FBS, 1% Antibiotic-Antimycotic, 1% Glutamax, 1% HEPES) or PBS.

Cytoplast siRNA Transfection

1 M cytoplasts were suspended with warm 1 ml A/A free α-MEM full medium (16.5% Premium FBS, 1% Glutamax, 1% HEPES). Two μl siRNA was diluted with warm opti-MEM and mixed with pipet at least 20 times. Eight μl lipofectamine-3000 was diluted with 92 μl warm opti-MEM and mixed with pipet at least 20 times. The ratio of siRNA and lipofectamine-3000 was 1:4 (v/v). The siRNA and lipofectamine-3000 dilutions were mixed with pipet at least 20 times and incubated at room temperature for 15 minutes. The siRNA and lipofectamine-3000 mixture was added to the cytoplast suspension, mixed well and incubated at 37° C. for 20 minutes. The suspension was shaken every 5 minutes to prevent cell clumping. After a 20-minute incubation, the cells were centrifuged, and re-suspended with normal α-MEM full medium (16.5% Premium FBS, 1% Antibiotic-Antimycotic, 1% Glutamax, 1% HEPES).

### Generation of Oncolytic Virus Infected Cytoplasts

One day before enucleation (usually 18 hours before enucleation), 2.5*10\hat{ }6 hTERT-MSCs were seeded on a 15-cm dish. Roughly two hours after seeding, the cells were washed once with PBS. Cells were then infected with oHSV-GFP (Imanis OV3001) at different MOIs (0.05 or 0.5 for example) with 8 mL serum free opti-MEM. Next, cells were incubated at 37° C. for 2 hours with occasionally shaking. The virus inoculum was then discarded. 20 mL pre-warmed full culture medium (α-MEM, 16.5% Premium FBS, 1% Antibiotic-Antimycotic, 1% Glutamax, 1% HEPES) was added to each well. The cells were incubated at 37° C. until enucleation.

### Lentivirus Overexpressing Functional Proteins in Cytoplasts

Target cells were plated in one well of 6-well plate at density of 1-2×10⁵ cells/well, or 10 cm plate with 0.5-1 M MSCs. The next day, the concentrated recombinant lentivirus was thawed in a 37° C. water bath and removed from the bath immediately once thawed. The cells were then washed with PBS 3 times. 200 μL serum free medium or 2 mL serum free medium (1:1250 SureENTRY) was added. The target cells were infected in a 6-well plate with MOI 10:1. The next day, the viral supernatant was removed, and the appropriate complete growth medium was added to the cells. After 72 hours incubation, the cells were subcultured into 2×100 mm dishes. The appropriate amount of selection drug (i.e., puromycin) was added for stable cell-line generation. 10-15 days after selection, clones were picked for expansion and were screened for positive ones. The selected positive clones were expanded for enucleation. Engineered cytoplasts were prepared as outlined above. The target protein expression on cytoplasts was determined by ordinary biochemical methods or functional assays, e.g., fluorescent activated cell sorting (FACS), western blot, or Boyden chamber assay.

Peptide Loading into Cytoplasts

1×10⁵/ml per well were plated onto a 4-chamber glass slide (LabTek II 4-chamber glass slide, 155383) in full MSC media DMEM 1X (Gibco 12561-056); 16.5% premium FBS (Atlanta Biologics S1150); 1% HEPES 1M (Gibco 15630-80); 1% Anti-Anti 100X (Gibco 15240-062); 1% Glutamax 100X (Gibco 35050-061)]. Cells were allowed to attach for at least 1 hour or overnight. Cells were then rinsed with PBS (Gibco 14190-144). Arg9(FAM) (10 mM, Anaspec, AS-61207) was diluted in full media to a total concentration of 1:100 (100 uM). Cytoplasts were then incubated for 1 to 2 hours and rinsed 3 times with PBS. Hoechst 33342 (Invitrogen) was added at a 1:5000 dilution in full media for at least 10 minutes. Cells were then washed with PBS and imaged by epifluorescent microscopy.

### Example 6. Manufacturing of Enucleated Cells with Fixed Angle Centrifugation

### Preparation of 2X MEM

2X MEM was prepared by mixing 10 mL of 10X MEM (Gibco, 11430-030, 2.94 mL of sodium bicarbonate (7.5%, Gibco, 25080-094), 1 mL of 100X Pen-Strep (Gibco, 15140-122), and 36 mL of ultrapure water (Invitrogen, 10977-015) for each 50 mL quantity. Then, Cytochalasin B (Sigma Aldrich, C6762) was added to the 2X MEM to a final concentration of 20 μg/mL.

### Preparation of 1X MEM

1X MEM was prepared by mixing equal amount of 2X MEM with Cytochalasin B and ultrapure water.

### Preparation of 25% Ficoll

25% Ficoll was prepared by mixing equal amounts of 2X MEM with Cytochalasin B and 50% Ficoll which was prepared by mixing equal amounts of Ficoll (in grams) and ultrapure water (in milliliters) and autoclaving. Refraction measured was between 1.3790-1.3810.

### Preparation of Ficoll Gradient

The Ficoll gradient and Ficoll fraction were made according to Table 3 and Table 4 and incubated overnight at 37° C., 5% CO₂ with loosen caps. (For 200 mL: 240 mL of 50% Ficoll, 400 mL of 2X MEM, 300 mL of 1X MEM).

TABLE 3
Non-limiting example of Ficoll gradient and volume for
enucleating the cells with the method described herein

| | 25% | 17% | 16% | 15% | 12.5% | Total |
|---|---|---|---|---|---|---|
| 25% Ficoll | 150 | 102 | 64 | 60 | 90 | 466 |
| 1X MEM | 0 | 48 | 36 | 40 | 90 | 214 |
| Total | 150 | 150 | 100 | 100 | 180 | |

TABLE 4
Non-limiting example of Ficoll fraction for enucleating
the cells with the method described herein

| | | Fractionation volume | |
|---|---|---|---|
| Fractionation order | Layer | [tube#] | |
| 1 | 25% | 35.7 | [1-8] |
| 2 | 17% | 35.7 | [8-15] |
| 3 | 16% | 8.93 | [15-16] |
| 4 | 15% | 8.93 | [16-17] |
| 5 | 12.5% | 35.7 | [17-25] |
| 6 | 12.5% with cells | 57.14 | [25-37] |
| 7 | 1X MEM | 17.86 | [37-40] |

### Enucleation of Mesenchymal Stromal Cells (MSCs)

One day before enucleating, MSCs were seeded at density of 2.95×10² per T75 flask to reach ideal cell density (0.0168×10²/cm²). CC40NX ultracentrifuge was loaded with 200 mL of 1X MEM (without Cytochalasin B), vacuumed, and warmed to 31° C. overnight. On the day of enucleation, cells were washed with PBS and detached with trypsin. The cells were resuspended with PBS buffer and glucose until enucleation. Right before enucleation, the cells were centrifuged in 300 g for 5 min, resuspended in 65 mL of 12.5% Ficoll, mixed well, and transferred through a 40 μm strainer into a fresh 50 mL tube.

Loading of the ultracentrifuge tubes was done with a peristaltic (10 rpm) in the following order: (1) 17.86 mL of 1X MEM; (2) cells in 57.14 mL of 12.5% Ficoll; (3) 35.71 mL of 12.5% Ficoll; (4) 8.93 mL of 15% Ficoll; (5) 8.93 mL of 16% Ficoll; (6) 35.71 mL of 17% Ficoll; and 35.71 mL of 25% Ficoll.

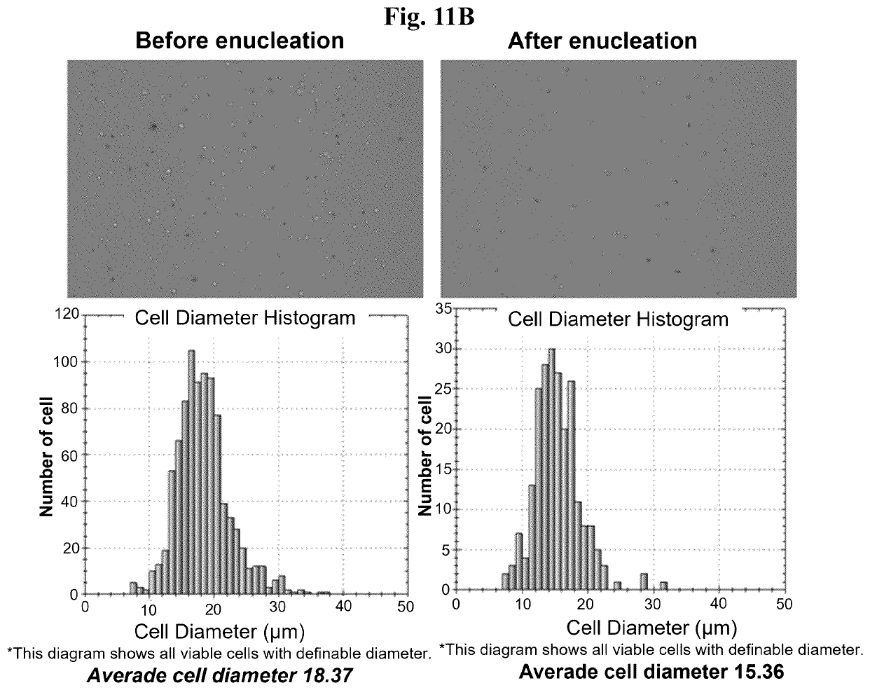

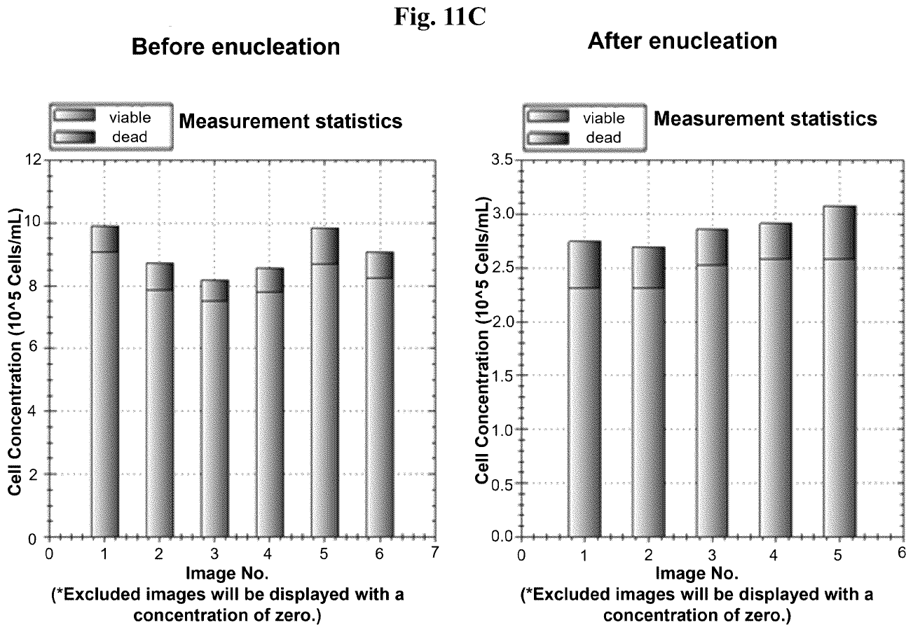

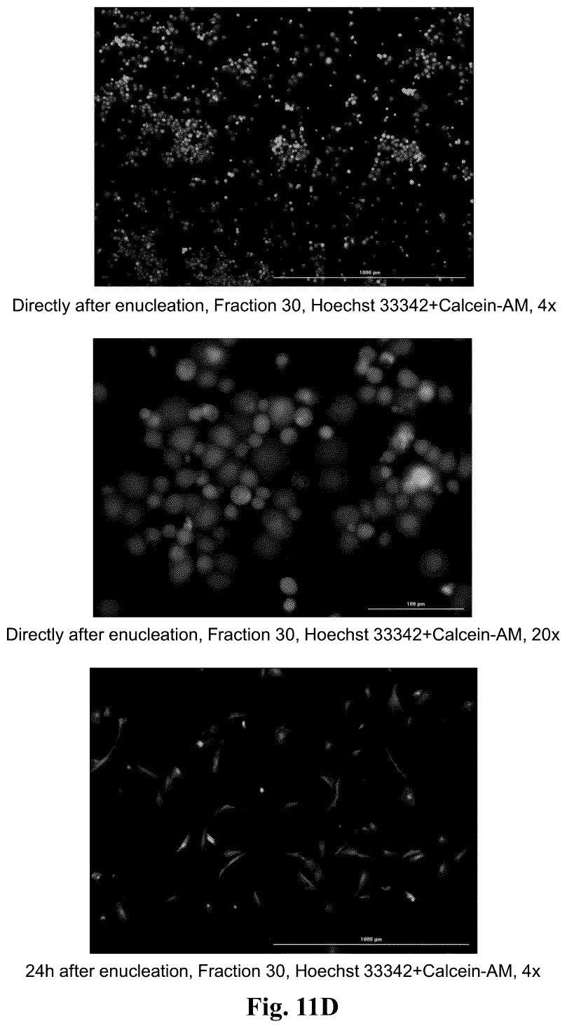

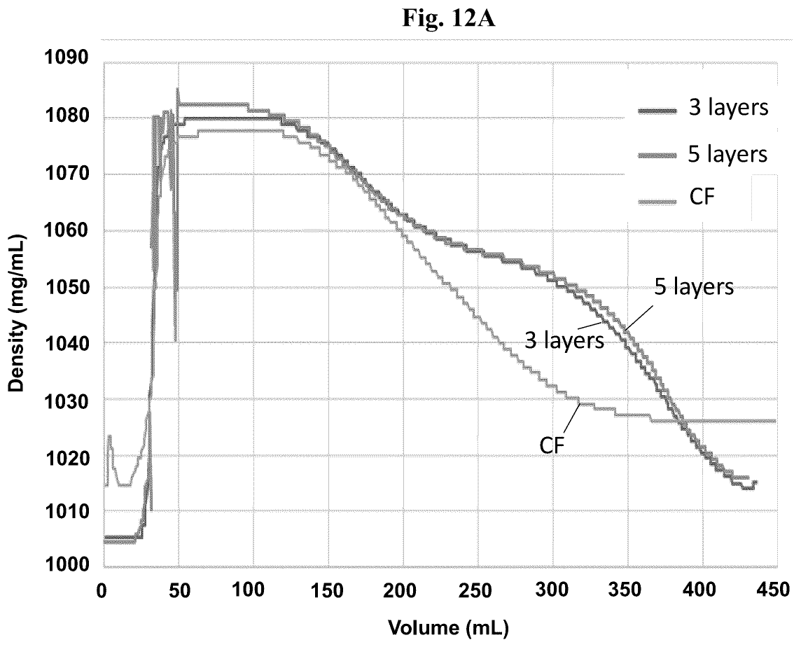

Centrifugation was done with a 30 min acceleration to a maximum speed of 36,000 rpm for another 30 min and minimal deceleration. When centrifugation was done, 200 mL was fractioned into 15 ml tubes containing 5 mL each. FIG. 11A illustrates an exemplary gradient generated by the method and the Ficoll fractions described herein during the acceleration and deceleration of the centrifugation. The density could be measured based on both Ficoll density (g/cm³) or BRIX (% of 1 g of sugar such as Ficoll per 100 g of aqueous solution).

Fractions containing enucleated cells were merged into one 50 mL tube, span down at 300 g for 6 min, and washed with warm serum-free αMEM. 35 mL was removed, and cells were resuspended again with serum-free αMEM. Then, cell viability, size, and enucleation efficiency were measured.

### Measurement of Cell Size and Viability

10 μL of cell suspension was diluted with 10 μL of Trypan Blue solution (0.4%) and mixed well. 10 μL of the mixture was loaded onto a cell counting slide. An automated cell counter was used to calculate cell size and viability. As seen in FIG. 11B, the diameter of the nucleated cell was decreased between before and after the enucleation. Furthermore, as seen in FIG. 11B and FIG. 11C the viability of cell was not significantly changed before or after enucleation.

### Measuring Enucleated Efficiency

50 μL of cell suspension was pipetted in a 96 well plate. It was stained with 1:2000 dilution of Hoechst 33342 (stains nuclei) and 1:500 dilution of Calcein AM and incubated at 37° C. for 5 min. Images of the cells were collected under a fluorescent microscope using the DAPI and FITC channels. FIG. 11D illustrates fluorescent images of the cells directly after enucleation (top two images) and 24 hours after enucleation (bottom image).

### Example 7. Generating Enucleated Cells by Zonal Centrifugation

### Preparation of 2X MEM

2X Modified Eagle Medium (MEM) is prepared by mixing 200 mL of 10X MEM (Gibco, 11430-030), 58.8 mL of sodium bicarbonate (7.5%, Gibco, 25080-094), 20 mL of 100X Pen-Strep (Gibco, 15140-122), and 720 mL of ultrapure water (Invitrogen, 10977-015) for each 1 L quantity. Then, 2X MEM is filtered through a 0.22 μm filter. 50 mL aliquots are made and stored at 4° C. for one month or until sedimentation is noticed.

### Preparation of Cytochalasin B Working Solution

One day before enucleation, 10 mg of Cytochalasin B (Sigma Aldrich, C6762) powder is dissolved in 1 mL of DMSO to generate a 10 mg/mL stock. 100 μL aliquots is prepared and stored at −20° C. until use. 100 μL of 10 mg/mL Cytochalasin B stock is added to 400 μL of DMSO to generate a 2 mg/mL working solution. Leftovers of the working solution can be stored at 4° C. for up to 2 weeks. 6 ml of 2 mg/mL of Cytochalasin B working solution is added to 600 mL of 2X MEM, creating a final solution containing 20 μL/mL of Cytochalasin B.

Preparation of 1X MEM without Cytochalasin B

1X MEM without Cytochalasin B is prepared in a 2 L bottle by adding 900 mL of 2X MEM without Cytochalasin B to 900 mL of ultrapure water.

Preparation of 1X MEM with Cytochalasin B

1X MEM with Cytochalasin B is prepared in a 1 L bottle by adding 270 mL of the final solution containing 20 μL/mL of Cytochalasin B to 270 mL of ultrapure water to generate a 1X MEM with Cytochalasin B working solution containing 10 μL/mL of Cytochalasin B.

### Preparation of 25% Ficoll

50% Ficoll is prepared by mixing equal amounts of Ficoll (in grams) and ultrapure water (in milliliters). 25% Ficoll is prepared in a 50 mL conical by mixing 520 mL of 2X MEM with Cytochalasin B and 520 mL of 50% Ficoll, creating a Ficoll working solution containing 10 μg/mL of Cytochalasin B. 25% Ficoll is vortexed for 1 min, then allow it to rest until bubbles dissipate. 70 μL of the 25% Ficoll is used to measure the refractive index. Refraction between 1.3790-1.3810 is measured. Optimal refractive index can be achieved by adding the final solution of 2X MEM with Cytochalasin B or mixing equal amounts of 50% Ficoll and 2X MEM with Cytochalasin B.

### Preparation of Enucleation Working Solution and Gradients

While the bubbles dissipate, 1X MEM with Cytochalasin B is added to 4 500 mL sterile bottles labeled 17%, 16%, 15%, and 12.5%, 102.4 mL of 1X MEM with Cytochalasin B is added to the 17% bottle; 28.8 mL of 1X MEM with Cytochalasin B is added to the 16% bottle; 32 mL of 1X MEM with Cytochalasin B is added to the 15% bottle; and 400 mL of 1X MEM with Cytochalasin B is added to the 12.5% bottle.

25% Ficoll is added to the 4 500 mL sterile bottles labeled 17% Ficoll, 16% Ficoll, 15% Ficoll, and 12.5% Ficoll. 217.6 mL of 25% Ficoll is added to the 17% bottle, 51.2 mL of 25% Ficoll is added to the 16% bottle, 48 mL of 25% Ficoll is added to the 15% bottle, and 400 mL of 25% Ficoll is added to the 12.5% bottle to make 17%, 16%, 15%, and 12.5% Ficoll layers.

Sterile bottles labeled 17%, 16%, 15%, and 12.5% with loosen caps are incubated overnight at 37° C. in a 5% CO₂ incubator.

### Enucleation of Cells

On the day of enucleation, the ultracentrifuge is preheated to 31° C. Serum-free αMEM is preheated in a 37° C. water bath. Cells detached from cell culturing surface are resuspend and mixed in 5 mL of 12.5% Ficoll. 452.6 mL of 12.5% Ficoll is added to the cell mixture for a total of 457.6 mL. Cells are strained through a 40 μM strainer. Cells are incubated for 30 minutes in a 37° C. incubator. To prepare Zonal centrifuge for acceleration to Zonal speed of 10000 relative centrifugal force (RCF), the rotor is filled with 1.6 L of 1X MEM using a peristaltic pump. After 30 minutes of incubation of cells, the layers are loaded into the centrifuge in the following order while the speed of the peristaltic pump is minimal: 457.6 mL of 12.5% Ficoll with cells; 286 mL of 12.5% Ficoll without cells; 71.5 mL of 15% Ficoll; 71.5 mL of 16% Ficoll; and 286 mL of 17% Ficoll, 286 mL of 25% Ficoll. The seal assembly is moved and capped and ran at 102000 RCF for one hour with the acceleration and deceleration set to minimum.

Once the run time is achieved, the rotor is decelerated to zonal speed (10000 RCF). The cap is removed and connected to the seal assembly. The extrusion solution is pushed from the outer wall and is collected in 10 mL or 50 mL fractions. Fractions are listed in Table 5. Once fractions are collected, the rotor is stopped and cleaned.

TABLE 5
Non-limited example of Ficoll fraction for enucleating
the cells with the method described herein

| Layer | | | | |
|---|---|---|---|---|
| fractionation | | | Fraction # | Fraction # |
| order | Layer | Volume [mL] | [10 ml] | [50 ml] |
| 1 | 25% | 286 | 1-29 | 1-6 |
| 2 | 17% | 572 | 29-58 | 6-12 |
| 3 | 16% | 643.5 | 58-65 | 12-13 |
| 4 | 15% | 715 | 65-72 | 13-15 |
| 5 | 12.5% | 1458.6 | 72-146 | 15-30 |

Fractions containing the enucleated cells are identified and spun down at 200 RCF for 10 minutes. The enucleated cells are washed with pre-warmed serum-free αMEM and then resuspend with pre-warmed serum-free αMEM.

The enucleated cells are counted using Trypan Blue. About 20,000 of the enucleated cells from each layer are added to a 96-well plate. PBS is added to bring the total volume to 45 μL. 1:500 diluted Hoechst containing 20 μg/mL Hoechst is diluted to an additional 1:10 when added to the enucleated cells, creating a final Hoechst concentration of 2 μg/mL.

The enucleated cells are incubated at 37° C. with 5% CO₂ for 10 minutes. The enucleated cells are imaged using a fluorescence microscope in order to determine the enucleation efficiency measured as cells without Hoechst staining (e.g., the enucleated cells). The enucleated cells can be processed for downstream experimentation or freezing.

### Example 8. Manufacturing of Enucleated Cells with Continuous Flow Centrifugation

This example illustrates enucleated cells with continuous flow centrifugation with density gradients. Cells were thawed in a 5 vial intervals: taking out 5 vials out of the −80C incubate for 45 seconds in 37° C. water bath; adding 1 ml of warm complete aMEM dropwise to each vial; transferring to a 50 ml tube; completing to a total of 50 ml complete aMEM; spinning down at 300 RCF for 5 minutes; and repeating with the next 5 vials. Supernatant was removed, and the cell pellet was resuspend in 1 ml of PBS. All 50 ml tubes were combined into two 50 ml tubes. PBS was added to reach a total of 50 ml in each tube. The cells were counted for each tube with

Tube 1: 3.21M/ml×50 ml=160 M, 91%, 14.42 um; and Tube 2: 2.68M/ml×50 ml=134M, 91%, 24.4 um for a total of 294 M (millions) cells. About 200,000 cells were kept as control.

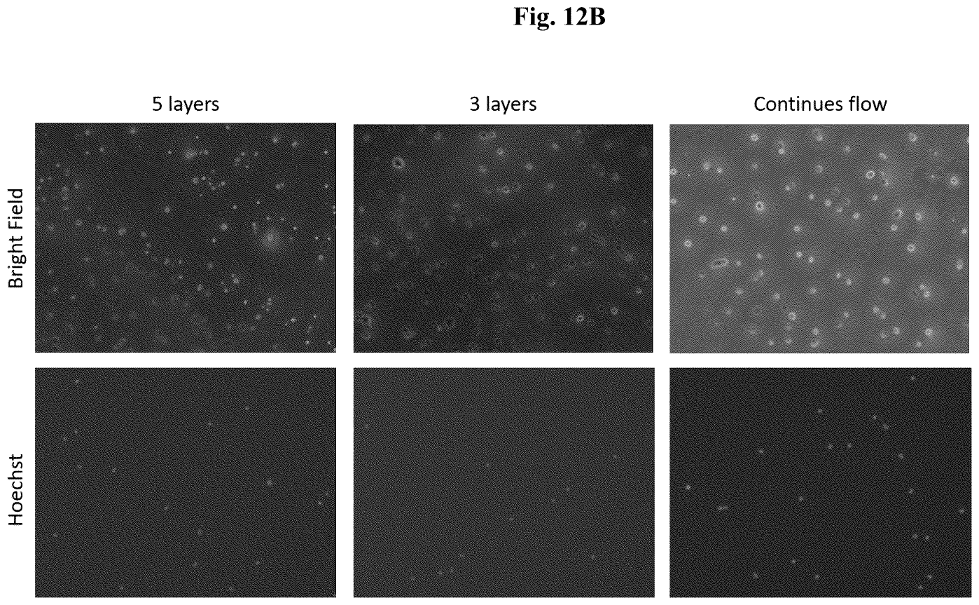

Measure the refraction index of the 25% Ficoll was determined to be 1.3794. Cytochalasin B was added to each Ficoll gradient (Stock=10 mg/ml, final concentration=10 ug/ml) based on Table 6. Cells were resuspended with 50 ml of 12.5% Ficoll with Cytochalasin B and transferred through a 100 um strainer for continuous flow centrifugation. FIG. 12A illustrates density gradient measured after an continuous flow centrifugation (done by ultracentrifugation run (5 Ficoll layers in medium gray, 3 Ficoll layers in dark gray and continuous flow as indicated by call-out line).

TABLE 6
Preparation of Ficoll gradient

| | Volume prepared | Volume | Volume |
|---|---|---|---|
| | in this | loaded into | Cytochalasin B |
| Layer | run [ml] | centrifuge | added [ul] |
| 12.5 | 120 | 114 | 100 |
| Cells | | | |
| 12.5 | 100 | 72 | 120 |
| 15 | 50 | 18 | 50 |
| 16 | 50 | 18 | 50 |
| 17 | 100 | 70 | 100 |
| 25 | 100 | 72 | 100 |

While centrifuge was running, the control nucleated cells were seeded as indicated in the bottom 96 well plate. 96 well plate and 6 well plates were coated with Fibronectin for 1 hour at 37° C. 10 ml fractions were collected into 15 ml tubes. Fractions (with visible cells) were chosen according to Table 7. Tubes from the same layer were merged into a single 50 ml tube and supplemented with warm serum free aMEM for a total of 50 ml. Supernatant was removed to the 10 ml line and wash again with 40 ml serum free aMEM. Cells were resuspended with warm complete aMEM and counted for seeding 20,000 cells per well. Cells were allowed to attach for 2 hours at 37° C. incubator. Unseeded cells were frozen with freezing media (90% serum+10% DMSO). Cells were then fixed with paraformaldehyde for Hoechst staining and imaging. FIG. 12B illustrate representatives images of enucleation efficiency test. Each field image was taken using bright field (total cells) and Hoechst channels (total nucleated cells). Table 8 illustrates that the enucleated cells were smaller as compared to nucleated cells. Table 8 also shows the yield (as determined by total number of nucleated cells divided by initial cell number and expressed in percentage) and enucleation efficacies.

TABLE 7
Fractions chosen for enucleated cells

| Fraction elution | | Fractions Volume | |
|---|---|---|---|
| order | Layer | (ml) [tubes #] | |
| 1 | 25% | 71.42 | [1-8] |
| 2 | 17% | 71.42 | [8-15] |
| 3 | 16% | 17.86 | [15-16] |
| 4 | 15% | 17.86 | [16-17] |
| 5 | 12.5% | 71.42 | [17-25] |
| 6 | 12.5% | 114.28 | [25-37] |
| | cells | | |
| 7 | 1X MEM | 25.71 | [37-40] |

| | | | | | | | Efficiency | Total | |
|---|---|---|---|---|---|---|---|---|---|
| Fraction | | | Cell | | | Cell | [fixed | Enucleated | |
| elution | Tubes # | Visible | concentration | Total cell | Viability | size | Hoechst | cell | Yield |
| order | merged | pellet? | [10\hat{ }6/ml] | number | % | [um] | cells] | [10\hat{ }6] | [%] |
| 1 | 2-7 | Yes | 0.29 | 0.29 | 14 | 13.34 | 50% | 73.35 | 24.9489795918 |
| 2 | 8-11 | No | | | | | | | |
| 3 | 12-14 | No | | | | | | | |
| 4 | 15-17 | Yes | 2.95 | 14.75 | 90 | 11.81 | 95% | | |
| 5 | 18-20 | Yes | 2.78 | 27.8 | 83 | 11.83 | 95% | | |
| 6 | 21-22 | Yes | 2.09 | 10.45 | 89 | 12.29 | 95% | | |
| 7 | 23-25 | Yes | 2.67 | 13.35 | 90 | 12.75 | 95% | | |
| 8 | 26-29 | Yes | 1.4 | 7 | 82 | 10.74 | 95% | | |
| 9 | 30-33 | Yes | 4.47 | 4.47 | 84 | 10.39 | 50% | | |
| | | | | | | | debris | | |
| 10 | 34-39 | Yes | 1.11 | 5.55 | 74 | 10.7 | 60% | | |
| | | | | | | | debris | | |

TABLE 8
Yield of obtaining enucleated cells from continuous flow centrifugation

| | Initial | Initial | Total | | | |
|---|---|---|---|---|---|---|
| | cell | nucleated | enucleated | Enucleated | | Enucleation |
| | number | cell size | cell | cell size | Yield | efficiency |
| Run type | [10\hat{ }6] | [μm] | [10\hat{ }6] | [μm] | [%] | [%] |
| 5 Ficoll | 294 | 18 | 73.3 | 12 | 24.9 | >95 |
| layers | | | | | | |
| 3 Ficoll | 196.5 | 22.8 | 24.6 | 11.1 | 12.5 | >95 |
| layers | | | | | | |
| Continuous | 298.5 | 21.7 | 39.8 | 12 | 13.3 | 50 |
| flow with 5 | | | | | | |
| Ficoll layers | | | | | | |

While the foregoing disclosure has been described in some detail for purposes of clarity and understanding, it will be clear to one skilled in the art from a reading of this disclosure that various changes in form and detail may be made without departing from the true scope of the disclosure. For example, all the techniques and apparatus described above may be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method for cell processing, the method comprising:
a) providing a composition comprising nucleated cells; and
b) enucleating a portion of the nucleated cells to produce an enucleated cell fraction using continuous flow centrifugation.

2. The method of claim 1, wherein the portion of the nucleated cells comprises greater than or equal to about 95% of the nucleated cells.

3. The method of claim 1, wherein the composition provided in a) has a volume comprising between more than or equal to about 500 mL to about 10000 mL.

4. The method of claim 1, wherein the continuous flow centrifugation generates a density gradient that separates the enucleated cell fraction from the nucleated cells in the composition.

5. The method of claim 4, wherein the density gradient comprises a polysaccharide density gradient.

6. The method of claim 4, wherein the density gradient comprises at least two, at least three, at least four, at least five, at least six, or at least seven ranges of the density gradient.

7. The method of claim 5, wherein the polysaccharide density gradient comprises about 25% polysaccharide, about 17% polysaccharide, about 16% polysaccharide, about 15% polysaccharide, or about 12.5% polysaccharide.

8. The method of claim 1, wherein the enucleated cell fraction produced by performing the continuous flow centrifugation once comprises more than or equal to about: 6×10⁷ of enucleated cells to 250×10⁷ of enucleated cells.

9. The method of claim 1, wherein the enucleating in b) further comprises generating the density gradient comprising centrifuging a polysaccharide at a maximum centrifugal force of between about 30000 RCF to about 200000 RCF.

10. The method of claim 1, wherein the continuous flow centrifugation generates zonal centrifugation for separating at least one enucleated cell from the nucleated cells.

11. The method of claim 10, wherein the zonal centrifugation separates the at least one enucleated cell from the nucleated cells based on size of the at least one enucleated cell or wherein the zonal centrifugation separates the at least one enucleated cell from the nucleated cells based on mass of the at least one enucleated cell.

12. (canceled)

13. The method of claim 4, wherein at least one density fraction is obtained from the density gradient, wherein the at least one density fraction comprises a mixed population of a subset of the nucleated cells and enucleated cells of the enucleated cell fraction.

14. The method of claim 13, wherein the mixed population comprises at least 70% of the enucleated cells.

15. The method of claim 1, wherein using the continuous flow centrifugation increases a yield of obtaining enucleated cells from nucleated cells by at least 0.1 fold, 0.2 fold, 0.5 fold, 1.0 fold, 2.0 fold, 5.0 fold, 10.0 fold, or more fold compared to a method of obtaining the enucleated cells from the nucleated cells by a method without using the continuous flow centrifugation.

16. The method of claim 1, wherein the nucleated cells comprise a heterologous polynucleotide.

17. The method of claim 16, wherein the method comprises inducing cell death of the nucleated cells that are not enucleated after b), wherein the cell death is induced by expressing a heterologous gene product encoded by the heterologous polynucleotide in the nucleated cells.

18.-31. (canceled)

32. The method of any one of previous claims, wherein the nucleated cells comprise stem cells, mesenchymal stromal cells, or immune cells.

33. (canceled)

34. (canceled)

35. (canceled)

36. (canceled)

37. The method of claim 1, wherein the enucleated cells lack a nucleus and comprise one or more intracellular organelles for synthesis or secretion of an exogenous polypeptide in absence of the nucleus.

38. (canceled)

39. The method of claim 37, wherein the exogenous polypeptide comprises a therapeutic agent.

40. The method of any one of claims 1-39, wherein the enucleated cells comprise at least one targeting moiety, fusogenic moiety, and/or immune evasion moiety.

41.-100. (canceled)
